(19) United States
(12) Patent Application Publication
(10) Pub. No.: US 2018/0346488 A1
(43) Pub. Date: Dec. 6, 2018

(54) CYTOTOXIC BENZODIAZEPINE DERIVATIVES AND CONJUGATES THEREOF

(71) Applicant: IMMUNOGEN, INC., Waltham, MA (US)

(72) Inventors: Michael Louis Miller, Framingham, MA (US); Manami Shizuka, Belmont, MA (US)

(21) Appl. No.: 15/956,848

(22) Filed: Apr. 19, 2018

Related U.S. Application Data
(60) Provisional application No. 62/487,573, filed on Apr. 20, 2017.

(51) Int. Cl. C07D 519/00; A61K 35/00

(52) U.S. Cl. CPC C07D 519/00; A61K 35/00

(57) ABSTRACT

The invention relates to novel benzodiazepine derivatives with antiproliferative activity and more specifically to novel benzodiazepine compounds of formulae (I) and (II). The invention also provides conjugates of the benzodiazepine compounds linked to a cell-binding agent. The invention further provides compositions and methods useful for inhibiting abnormal cell growth or treating a proliferative disorder in a mammal using the compounds or conjugates of the invention.

### RELATED APPLICATION

This application claims the benefit of the filing date, under 35 U.S.C. § 119(e), of U.S. Provisional Application No. 62/487,573, filed on Apr. 20, 2017, the entire contents of which are incorporated herein by reference.

### FIELD OF THE INVENTION

The present invention relates to novel cytotoxic compounds, and cytotoxic conjugates comprising these cytotoxic compounds and cell-binding agents. More specifically, this invention relates to novel benzodiazepine compounds, derivatives thereof, intermediates thereof, conjugates thereof, and pharmaceutically acceptable salts thereof, which are useful as medicaments, in particular as anti-proliferative agents.

### BACKGROUND OF THE INVENTION

Benzodiazepine derivatives are useful compounds for treating various disorders, and include medicaments such as, antiepileptics (imidazo [2,1-b][1,3,5]benzothiadiazepines, U.S. Pat. No. 4,444,688; U.S. Pat. No. 4,062,852), antibacterials (pyrimido[1,2-c][1,3,5]benzothiadiazepines, GB 1476684), diuretics and hypotensives (pyrrolo(1,2-b)[1,2,5]benzothiadiazepine 5,5 dioxide, U.S. Pat. No. 3,506,646), hypolipidemics (WO 03091232), anti-depressants (U.S. Pat. No. 3,453,266); osteoporosis (JP 2138272).

It has been shown in animal tumor models that benzodiazepine derivatives, such as pyrrolobenzodiazepines (PBDs), act as anti-tumor agents (N-2-imidazolyl alkyl substituted 1,2,5-benzothiadiazepine-1,1-dioxide, U.S. Pat. No. 6,156,746), benzo-pyrido or dipyrido thiadiazepine (WO 2004/069843), pyrrolo [1,2-b] [1,2,5]benzothiadiazepines and pyrrolo[1,2-b][1,2,5] benzodiazepine derivatives (WO2007/015280), tomaymycin derivatives (e.g., pyrrolo[1,4]benzodiazepines), such as those described in WO 00/12508, WO2005/085260, WO2007/085930, and EP 2019104. Benzodiazepines are also known to affect cell growth and differentiation (Kamal A., et al., Bioorg. Med. Chem., 2008 Aug. 15; 16(16):7804-10 (and references cited therein); Kumar R, Mini Rev Med Chem. 2003 June; 3(4):323-39 (and references cited therein); Bednarski J J, et al., 2004; Sutter A. P, et al., 2002; Blatt N B, et al., 2002), Kamal A. et al., Current Med. Chem., 2002; 2; 215-254, Wang J-J., J. Med. Chem., 2206; 49:1442-1449, Alley M. C. et al., Cancer Res. 2004; 64:6700-6706, Pepper C. J., Cancer Res 2004; 74:6750-6755, Thurston D. E. and Bose D. S., Chem. Rev., 1994; 94:433-465; and Tozuka, Z., et al., Journal of Antibiotics, (1983) 36; 1699-1708. General structure of PBDs is described in US Publication Number 20070072846. The PBDs differ in the number, type and position of substituents, in both their aromatic A rings and pyrrolo C rings, and in the degree of saturation of the C ring. Their ability to form an adduct in the minor groove and crosslink DNA enables them to interfere with DNA processing, hence their potential for use as antiproliferative agents.

The first pyrrolobenzodiazepine to enter the clinic, SJG-136 (NSC 694501) is a potent cytotoxic agent that causes DNA inter-strand crosslinks (S. G Gregson et al., 2001, J. Med. Chem., 44: 737-748; M. C. Alley et al., 2004, Cancer Res., 64: 6700-6706; J. A. Hartley et al., 2004, Cancer Res., 64: 6693-6699; C. Martin et al., 2005, Biochemistry., 44: 4135-4147; S. Amould et al., 2006, Mol. Cancer Ther., 5: 1602-1509). Results from a Phase I clinical evaluation of SJG-136 revealed that this drug was toxic at extremely low doses (maximum tolerated dose of 45 μg/m², and several adverse effects were noted, including vascular leak syndrome, peripheral edema, liver toxicity and fatigue. DNA damage was noted at all doses in circulating lymphocytes (D. Hochhauser et al., 2009, Clin. Cancer Res., 15: 2140-2147).

Thus, there exists a need for improved benzodiazepine derivatives that are less toxic and still therapeutically active for treating a variety of proliferative diseases, such as cancer.

### SUMMARY OF THE INVENTION

In a first aspect, the present invention is directed to a cyctotoxic compound represented by the following formula:

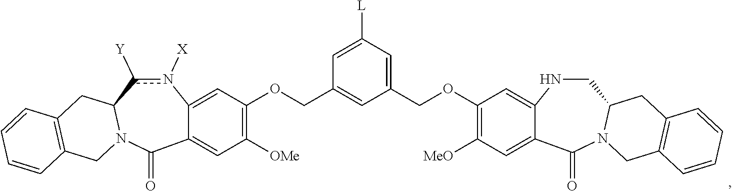

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;

L is represented by the following formula:

—NR₅—P—C(═O)—W-J (L1);

—NR₅—P—C(═O)—W—S—Z^{s} (L2);

—N(R^{e′})—W—S—Z^{s} (L3);

—N(R^{e})—C(═O)—W—S—Z^{s} (L4); or

—N(R^{e′})—W-J (L5);

R₅, for each occurrence, is independently H or a (C₁-C₃)alkyl;

W is a spacer unit;

J is a reactive moiety capable of forming a covalent bond with a cell-binding agent;

R^{e} is H or a (C₁-C₃)alkyl;

R^{e′} is —(CH₂—CH₂—O)ₙ—R^{k};

n is an integer from 2 to 6;

R^{k} is H or Me;

Z^{s} is H, —SR^{d}, —C(═O)R^{d1} or a bifunctional linker having a reactive moiety capable of forming a covalent bond with a cell-binding agent;

R^{d} is a (C₁-C₆)alkyl or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and

R^{d1} is a (C₁-C₆)alkyl.

In a second aspect, the present invention is directed to a cell-binding agent-cytotoxic agent conjugate represented by the following formula:

CBACy)ₚ (III),

or a pharmaceutically acceptable salt thereof, wherein:

CBA is a cell-binding agent;

Cy is a cytotoxic agent represented by the following formula:

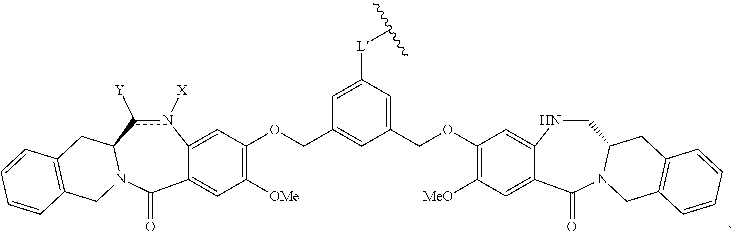

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;

L′ is represented by the following formula:

—NR₅—P—C(═O)—W-J′ (L1′);

—NR₅—P—C(═O)—W—S—Z^{s1} (L2′);

—N(R^{e′})—W—S—Z^{s1} (L3′);

—N(R^{e})—C(═O)—W—S—Z^{s1} (L4′); or

—N(R^{e})—W-J′ (L5′);

R₅, for each occurrence, is independently H or a (C₁-C₃)alkyl;

W is a spacer unit;

J′ is a linking moiety;

R^{e} is H or a (C₁-C₃)alkyl;

R^{e′} is —(CH₂—CH₂—O)ₙ—R^{k};

n is an integer from 2 to 6;

R^{k} is H or Me;

Z^{s1} is a bifunctional linker covalently linked to the cytotoxic agent and the CBA;

p is an integer from 1 to 20

The present invention also includes a composition (e.g., a pharmaceutical composition) comprising novel benzodiazepine compounds, derivatives thereof, or conjugates thereof, (and/or solvates, hydrates and/or salts thereof) and a carrier (a pharmaceutically acceptable carrier). The present invention additionally includes a composition (e.g., a pharmaceutical composition) comprising novel benzodiazepine compounds, derivatives thereof, or conjugates thereof (and/or solvates, hydrates and/or salts thereof), and a carrier (a pharmaceutically acceptable carrier), further comprising a second therapeutic agent. The present compositions are useful for inhibiting abnormal cell growth or treating a proliferative disorder in a mammal (e.g., human). The present compositions are useful for treating conditions such as cancer, rheumatoid arthritis, multiple sclerosis, graft versus host disease (GVHD), transplant rejection, lupus, myositis, infection, immune deficiency such as AIDS, and inflammatory diseases in a mammal (e.g., human).

The present invention includes a method of inhibiting abnormal cell growth or treating a proliferative disorder in a mammal (e.g., human) comprising administering to said mammal a therapeutically effective amount of novel benzodiazepine compounds, derivatives thereof, or conjugates thereof, (and/or solvates and salts thereof) or a composition thereof, alone or in combination with a second therapeutic agent. In some embodiments, the proliferative disorder is cancer. Also included in the present invention is the use of the novel benzodiazepine compounds, derivatives thereof, or conjugates thereof, (and/or solvates and salts thereof) or a composition thereof for the manufacture of a medicament for inhibiting abnormal cell growth or treating a proliferative disorder (e.g., cancer) in a mammal (e.g., human).

The present invention includes a method of synthesizing and using novel benzodiazepine compounds, derivatives thereof, and conjugates thereof for in vitro, in situ, and in vivo diagnosis or treatment of mammalian cells, organisms, or associated pathological conditions.

### BRIEF DESCRIPTION OF THE FIGURES

FIGS. 1-3 show mass spectra of exemplary deglycosylated conjugates of the present invention.

FIGS. 4 and 5 show individual body weight and body weight changes for female CD-1 mice treated with 100 or 200 μg/kg of M9346A-30 conjugate.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention.

It should be understood that any of the embodiments described herein, including those described under different aspects of the invention (e.g., compounds, compound-linker molecules, conjugates, compositions, methods of making and using) and different parts of the specification (including embodiments described only in the Examples) can be combined with one or more other embodiments of the invention, unless explicitly disclaimed or improper. Combination of embodiments are not limited to those specific combinations claimed via the multiple dependent claims.

### Definitions

As used herein, the term “treating” or “treatment” includes reversing, reducing, or arresting the symptoms, clinical signs, and underlying pathology of a condition in manner to improve or stabilize a subject's condition. As used herein, and as well understood in the art “treatment” is an approach for obtaining beneficial or desired results, including clinical results. Beneficial or desired clinical results can include, but are not limited to, alleviation, amelioration, or slowing the progression, of one or more symptoms or conditions associated with a condition, e.g., cancer, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein

As used herein, the term “cell-binding agent” or “CBA” refers to a compound that can bind a cell (e.g., on a cell-surface ligand) or bind a ligand associated with or proximate to the cell, preferably in a specific manner. In certain embodiments, binding to the cell or a ligand on or near the cell is specific. The CBA may include peptides and non-peptides.

“Linear or branched alkyl” as used herein refers to a saturated linear or branched-chain monovalent hydrocarbon radical. In preferred embodiments, a straight chain or branched chain alkyl has thirty or fewer carbon atoms in its backbone (e.g., C₁-C₃₀ for straight chains, C₃-C₃₀ for branched chains), and more preferably twenty or fewer. Examples of alkyl include, but are not limited to, methyl, ethyl, 1-propyl, 2-propyl, 1-butyl, 2-methyl-1-propyl, —CH₂CH(CH₃)₂), 2-butyl, 2-methyl-2-propyl, 1-pentyl, 2-pentyl 3-pentyl, 2-methyl-2-butyl, 3-methyl-2-butyl, 3-methyl-1-butyl, 2-methyl-1-butyl, 1-hexyl), 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like.

Moreover, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. In certain embodiments, a straight chain or branched chain alkyl has or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In other embodiments, the chain has six or fewer carbon (C₁-C₆) atoms in its backbone.

“Linear or branched alkenyl” refers to linear or branched-chain monovalent hydrocarbon radical of two to twenty carbon atoms with at least one site of unsaturation, i.e., a carbon-carbon, double bond, wherein the alkenyl radical includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like. Preferably, the alkenyl has two to ten carbon atoms. More preferably, the alkyl has two to four carbon atoms.

“Linear or branched alkynyl” refers to a linear or branched monovalent hydrocarbon radical of two to twenty carbon atoms with at least one site of unsaturation, i.e., a carbon-carbon, triple bond. Examples include, but are not limited to, ethynyl, propynyl, 1-butynyl, 2-butynyl, 1-pentynyl, 2-pentynyl, 3-pentynyl, hexynyl, and the like. Preferably, the alkynyl has two to ten carbon atoms. More preferably, the alkynyl has two to four carbon atoms.

The term “carbocycle,” “carbocyclyl” and “carbocyclic ring” refer to a monovalent non-aromatic, saturated or partially unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo [4,5], [5,5], [5,6], or [6,6] system, and bicyclic carbocycles having 9 or 10 ring atoms can be arranged as a bicyclo [5,6] or [6,6] system, or as bridged systems such as bicyclo[2.2.1]heptane, bicyclo [2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

The terms “cyclic alkyl” and “cycloalkyl” can be used interchangeably. As used herein, the term refers to the radical of a saturated ring. In preferred embodiments, cycloalkyls have from 3-10 carbon atoms in their ring structure, and more preferably from 5-7 carbon atoms in the ring structure. In some embodiments, the two cyclic rings can have two or more atoms in common, e.g., the rings are “fused rings.” Suitable cycloalkyls include cycloheptyl, cyclohexyl, cyclopentyl, cyclobutyl and cyclopropyl.

In some embodiments, the cycloalkyl is a mono-cyclic group. In some embodiments, the cycloalkyl is a bi-cyclic group. In some embodiments, the cycloalkyl is a tri-cyclic group.

The term “cyclic alkenyl” refers to a carbocyclic ring radical having at least one double bond in the ring structure.

The term “cyclic alkynyl” refers to a carbocyclic ring radical having at least one triple bond in the ring structure.

The term “aryl” as used herein, include substituted or unsubstituted single-ring aromatic groups in which each atom of the ring is carbon. Preferably the ring is a 5- to 7-membered ring, more preferably a 6-membered ring. Aryl groups include phenyl, phenol, aniline, and the like. The terms “aryl” also includes “polycyclyl”, “polycycle”, and “polycyclic” ring systems having two or more rings in which two or more atoms are common to two adjoining rings, e.g., the rings are “fused rings,” wherein at least one of the rings is aromatic, e.g., the other cyclic rings can be cycloalkyis, cycloalkenyls, cycloalkynyis. In some preferred embodiments, polycycles have 2-3 rings. In certain preferred embodiments, polycyclic ring systems have two cyclic rings in which both of the rings are aromatic. Each of the rings of the polycycle can be substituted or unsubstituted. In certain embodiments, each ring of the polycycle contains from 3 to 10 atoms in the ring, preferably from 5 to 7. For example, aryl groups include, but are not limited to, phenyl (benzene), tolyl, anthracenyl, fluorenyl, indenyl, azulenyl, and naphthyl, as well as benzo-fused carbocyclic moieties such as 5,6,7,8-tetrahydronaphthyl, and the like In some embodiments, the aryl is a single-ring aromatic group. In some embodiments, the aryl is a two-ring aromatic group. In some embodiments, the aryl is a three-ring aromatic group.

The terms “heterocycle,” “heterocyclyl,” and “heterocyclic ring” as used herein, refers to substituted or unsubstituted non-aromatic ring structures of 3- to 18-membered rings, preferably 3- to 10-membered rings, more preferably 3- to 7-membered rings, whose ring structures include at least one heteroatom, preferably one to four heteroatons, more preferably one or two heteroatoms. In certain embodiments, the ring structure can have two cyclic rings. In some embodiments, the two cyclic rings can have two or more atoms in common, e.g., the rings are “fused rings.” Heterocyclyl groups include, for example, piperidine, piperazine, pyrrolidine, morpholine, lactones, lactams, and the like. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Examples of heterocyclic rings include, but are not limited to, tetrahydrofurane, dihydrofurane, tetrahydrothiene, tetrahydropyrane, dihydropyrane, tetrahydrothiopyranyl, thiomorpholine, thioxane, homopiperazine, azetidine, oxetane, thietane, homopiperidine, oxepane, thiepane, oxazepine, diazepine, thiazepine, 2-pyrroline, 3-pyrroline, indoline, 2H-pyrane, 4H-pyrane, dioxanyl, 1,3-dioxolane, pyrazoline, dithiane, dithiolane, dihydropyrane, dihydrothiene, dihydrofurane, pyrazolidinylimidazoline, imidazolidine, 3-azabicyco[3.1.0]hexanyl, 3-azabicyclo[4.1.0]heptane, and azabicyclo[2.2.2]hexane. Spiro moieties are also included within the scope of this definition. Examples of a heterocyclic group wherein ring atoms are substituted with oxo (═O) moieties are pyrimidinone and 1, 1-dioxo-thiomorpholine.

The term “heteroaryl” as used herein, refers to substituted or unsubstituted aromatic single ring structures, preferably 5- to 7-membered rings, more preferably 5- to 6-membered rings, whose ring structures include at least one heteroatom (e.g., O, N, or S), preferably one to four or one to 3 heteroatoms, more preferably one or two heteroatoms. When two or more heteroatoms are present in a heteroaryl ring, they may be the same or different. The term “heteroaryl” also includes “polycyclyl”, “polycycle”, and “polycyclic” ring systems having two or more cyclic rings in which two or more carbons are common to two adjoining rings, e.g., the rings are “fused rings,” wherein at least one of the rings is heteroaromatic, e.g., the other cyclic rings can be cycloalkyls, cycloalkenyls, cycloalkynyls, aryls, and/or heterocyclyls. In some preferred embodiments, preferred polycycles have 2-3 rings. In certain embodiments, preferred polycyclic ring systems have two cyclic rings in which both of the rings are aromatic. In certain embodiments, each ring of the polycycle contains from 3 to 10 atoms in the ring, preferably from 5 to 7. For examples, heteroaryl groups include, but are not limited to, pyrrole, furan, thiophene, imidazole, oxazole, thiazole, pyrazole, pyridine, pyrazine, pyridazine, quinoline, pyrimidine, indolizine, indole, indazole, benzimidazole, benzothiazole, benzofuran, benzothiophene, cinnoline, phthalazine, quinazoline, carbazole, phenoxazine, quinoline, purine and the like.

In some embodiments, the heteroaryl is a single-ring aromatic group. In some embodiments, the heteroaryl is a two-ring aromatic group. In some embodiments, the heteroaryl is a three-ring aromatic group.

The heterocycle or heteroaryl groups may be carbon (carbon-linked) or nitrogen (nitrogen-linked) attached where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or O-carboline.

The heteroatoms present in heteroaryl or heterocyclcyl include the oxidized forms such as NO, SO, and SO₂.

The term “halo” or “halogen” refers to fluorine (F), chlorine (Cl), bromine (Br) or iodine (I).

The alkyl, alkenyl, alkynyl, cyclic alkyl, cyclic alkenyl, cyclic alkynyl, carbocyclyl, aryl, heterocyclyl and heteroaryl described above can be optionally substituted with one more (e.g., 2, 3, 4, 5, 6 or more) substituents.

Unless specifically stated as “unsubstituted,” references to chemical moieties herein are understood to include substituted variants. For example, reference to an “alkyl” group or moiety implicitly includes both substituted and unsubstituted variants.

Examples of substituents on chemical moieties includes but is not limited to, halogen, hydroxyl, carbonyl (such as carboxyl, alkoxycarbonyl, formyl, or acyl), thiocarbonyl (such as thioester, thioacetate, or thioformate), alkoxyl, alkylthio, acyloxy, phosphoryl, phosphate, phosphonate, amino, amido, amidine, imine, cyano, nitro, azido, sulfhydryl, alkylthio, sulfate, sulfonate, sulfamoyl, sulfonamido, sulfonyl, heterocyclyl, aralkyl, or aryl or heteroaryl moiety.

The term “substituted” refers to moieties having substituents replacing a hydrogen on one or more carbons of the backbone of a chemical compound. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a fonnyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfbydryl, an alkylthio, a sulfate, a sulfonate, a sulfamoyl, a sulfonamido, a sulfonyl, a heterocyclyl, an aralkyl, or an aromatic or heteroaromatic moiety. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

“Optional” or “optionally” means that the subsequently described circumstance may or may not occur, so that the application includes instances where the circumstance occurs and instances where it does not. For example, the phrase “optionally substituted” means that a nonhydrogen substituent may or may not be present on a given atom, and, thus, the application includes structures wherein a non-hydrogen substituent is present and structures wherein a nonhydrogen substituent is not present.

If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R″, wherein R′ and R″ together with the nitrogen atom to which they are attached, may form a heterocyclic ring. The heterocyclic ring formed from R′ and R″ together with the nitrogen atom to which they are attached may be partially or fully saturated. In some embodiments, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. Such substituents, in non-limiting examples, can be selected from a linear, branched or cyclic alkyl, alkenyl or alkynyl having from 1 to 10 carbon atoms, aryl, heteroaryl, heterocyclyl, halogen, guanidinium [—NH(C═NH)NH₂], —OR¹⁰¹, NR¹⁰²R¹⁰³, —NO₂, —NR¹⁰²COR¹⁰³, —SR¹⁰¹, a sulfoxide represented by —SOR¹⁰¹, a sulfone represented by —SO₂R¹⁰¹, a sulfonate —SO₃M, a sulfate —OSO₃M, a sulfonamide represented by —SO₂NR¹⁰²R¹⁰³, cyano, an azido, —COR¹⁰¹, —OCOR¹⁰¹, —OCONR10²R¹⁰³ and a polyethylene glycol unit (—CH₂CH₂O)ₙR¹⁰¹ wherein M is H or a cation (such as Na⁺ or K⁺); R¹⁰¹, R¹⁰² and R¹⁰³ are each independently selected from H, linear, branched or cyclic alkyl, alkenyl or alkynyl having from 1 to 10 carbon atoms, a polyethylene glycol unit (—CH₂CH₂O)ₙ—R¹⁰⁴ wherein n is an integer from 1 to 24, an aryl having from 6 to 10 carbon atoms, a heterocyclic ring having from 3 to 10 carbon atoms and a heteroaryl having 5 to 10 carbon atoms; and R¹⁰⁴ is H or a linear or branched alkyl having 1 to 4 carbon atoms, wherein the alkyl, alkenyl, alkynyl, aryl, heteroaryl and heterocyclyl in the groups represented by R¹⁰¹, R¹⁰², R¹⁰³ and R¹⁰⁴ are optionally substituted with one or more (e.g., 2, 3, 4, 5, 6 or more) substituents independently selected from halogen, —OH, —CN, —NO₂ and unsubstituted linear or branched alkyl having 1 to 4 carbon atoms. Preferably, the substituents for the optionally substituted alkyl, alkenyl, alkynyl, cyclic alkyl, cyclic alkenyl, cyclic alkynyl, carbocyclyl, aryl, heterocyclyl and heteroaryl described above include halogen, —CN, —NR¹⁰²R¹⁰³, —CF₃, —OR¹, aryl, heteroaryl, heterocyclyl, —SR¹⁰¹, —SOR¹⁰¹, —SO₂R¹⁰¹ and —SO₃M.

The term “compound” or “cytotoxic compound,” “cytotoxic dimer” and “cytotoxic dimer compound” are used interchangeably. They are intended to include compounds for which a structure or formula or any derivative thereof has been disclosed in the present invention or a structure or formula or any derivative thereof that has been incorporated by reference. The term also includes, stereoisomers, geometric isomers, tautomers, solvates, metabolites, salts (e.g., pharmaceutically acceptable salts) and prodrugs, and prodrug salts of a compound of all the formulae disclosed in the present invention. The term also includes any solvates, hydrates, and polymorphs of any of the foregoing. The specific recitation of “stereoisomers,” “geometric isomers,” “tautomers,” “solvates,” “metabolites,” “salt” “prodrug,” “prodrug salt,” “conjugates,” “conjugates salt,” “solvate,” “hydrate,” or “polymorph” in certain aspects of the invention described in this application shall not be interpreted as an intended omission of these forms in other aspects of the invention where the term “compound” is used without recitation of these other forms.

The term “conjugate” as used herein refers to a compound described herein or a derivative thereof that is linked to a cell binding agent.

The term “linkable to a cell binding agent” as used herein refers to the compounds described herein or derivates thereof comprising at least one linking group or a precursor thereof suitable to bond these compounds or derivatives thereof to a cell binding agent.

The term “precursor” of a given group refers to any group which may lead to that group by any deprotection, a chemical modification, or a coupling reaction.

The term “linked to a cell binding agent” refers to a conjugate molecule comprising at least one of the compounds described herein, or derivative thereof bound to a cell binding agent via a suitable linking group or a precursor thereof.

The term “chiral” refers to molecules which have the property of non-superimposability of the mirror image partner, while the term “achiral” refers to molecules which are superimposable on their mirror image partner.

The term “stereoisomer” refers to compounds which have identical chemical constitution and connectivity, but different orientations of their atoms in space that cannot be interconverted by rotation about single bonds.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as crystallization, electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “prodrug” as used in this application refers to a precursor or derivative form of a compound of the invention that is capable of being enzymatically or hydrolytically activated or converted into the more active parent form. See, e.g., Wilman, “Prodrugs in Cancer Chemotherapy” Biochemical Society Transactions, 14, pp. 375-382, 615^{th} Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, ester-containing prodrugs, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of the invention and chemotherapeutic agents such as described above.

The term “prodrug” is also meant to include a derivative of a compound that can hydrolyze, oxidize, or otherwise react under biological conditions (in vitro or in vivo) to provide a compound of this invention. Prodrugs may only become active upon such reaction under biological conditions, or they may have activity in their unreacted forms. Examples of prodrugs contemplated in this invention include, but are not limited to, analogs or derivatives of compounds of any one of the formulae disclosed herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5^{th} ed.); see also Goodman and Gilman's, The Pharmacological basis of Therapeutics, 8^{th} ed., McGraw-Hill, Int. Ed. 1992, “Biotransformation of Drugs.”

One preferred form of prodrug of the invention includes compounds (with or without any linker groups) and conjugates of the invention comprising an adduct formed between an imine bond of the compounds/conjugates and an imine reactive reagent. Another preferred form of prodrug of the invention includes compounds such as those of formula (I) and (II), wherein when the double line [symbol] between N and C represents a single bond, X is H or an amine protecting group, and the compound becomes a prodrug. A prodrug of the invention may contain one or both forms of prodrugs described herein (e.g., containing an adduct formed between an imine bond of the compounds/conjugates and an imine reactive reagent, and/or containing a Y leaving group when X is —H).

The term “imine reactive reagent” refers to a reagent that is capable of reacting with an imine group. Examples of imine reactive reagent includes, but is not limited to, sulfites (H₂SO₃, H₂SO₂ or a salt of HSO₃⁻, SO₃²⁻ or HSO₂⁻ formed with a cation), metabisulfite (H₂S₂O₅ or a salt of S₂O₅²⁻ formed with a cation), mono, di, tri, and tetra-thiophosphates (PO₃SH₃, PO₂S₂H₃, POS₃H₃, PS₄H₃ or a salt of PO₃S³⁻, PO₂S₂³⁻, POS₃³⁻ or PS₄³⁻ formed with a cation), thio phosphate esters ((RⁱO)₂PS(ORⁱ), RⁱSH, RⁱSOH, RⁱSO₂H, RⁱSO₃H), various amines (hydroxyl amine (e.g., NH₂OH), hydrazine (e.g., NH₂NH₂), NH₂O—Rⁱ, R^{i′} NH—Rⁱ, NH₂—Rⁱ), NH₂—CO—NH₂, NH₂—C(═S)—NH_{2′} thiosulfate (H₂S₂O₃ or a salt of S₂O₃²⁻ formed with a cation), dithionite (H₂S₂O₄ or a salt of S₂O₄²⁻ formed with a cation), phosphorodithioate (P(═S)(OR^{k})(SH)(OH) or a salt thereof formed with a cation), hydroxamic acid (R^{k}C(═O)NHOH or a salt formed with a cation), hydrazide (R^{k}CONHNH₂), formaldehyde sulfoxylate (HOCH₂SO₂H or a salt of HOCH₂SO₂⁻ formed with a cation, such as HOCH₂SO₂⁻Na⁺), glycated nucleotide (such as GDP-mannose), fludarabine or a mixture thereof, wherein Rⁱ and R^{i′} are each independently a linear or branched alkyl having 1 to 10 carbon atoms and are substituted with at least one substituent selected from —N(R^{j})₂, —CO₂H, —SO₃H, and —PO₃H; Rⁱ and R^{i′} can be further optionally substituted with a substituent for an alkyl described herein; R^{j} is a linear or branched alkyl having 1 to 6 carbon atoms; and R^{k} is a linear, branched or cyclic alkyl, alkenyl or alkynyl having 1 to 10 carbon atoms, aryl, heterocyclyl or heteroaryl (preferably, R^{k} is a linear or branched alkyl having 1 to 4 carbon atoms; more preferably, R^{k} is methyl, ethyl or propyl). Preferably, the cation is a monovalent cation, such as N⁺ or K⁺. Preferably, the imine reactive reagent is selected from sulfites, hydroxyl amine, urea and hydrazine. More preferably, the imine reactive reagent is NaHSO₃ or KHSO₃.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the term “solvate” means a compound which further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

The terms “abnormal cell growth” and “proliferative disorder” are used interchangeably in this application. “Abnormal cell growth,” as used herein, unless otherwise indicated, refers to cell growth that is independent of normal regulatory mechanisms (e.g., loss of contact inhibition). This includes, for example, the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs.

The terms “cancer” and “cancerous” refer to or describe the physiological condition in mammals that is typically characterized by unregulated cell growth. A “tumor” comprises one or more cancerous cells, and/or benign or pre-cancerous cells.

A “therapeutic agent” encompasses both a biological agent such as an antibody, a peptide, a protein, an enzyme or a chemotherapeutic agent.

A “chemotherapeutic agent” is a chemical compound useful in the treatment of cancer.

A “metabolite” is a product produced through metabolism in the body of a specified compound, a derivative thereof, or a conjugate thereof, or salt thereof. Metabolites of a compound, a derivative thereof, or a conjugate thereof, may be identified using routine techniques known in the art and their activities determined using tests such as those described herein. Such products may result for example from the oxidation, hydroxylation, reduction, hydrolysis, amidation, deamidation, esterification, deesterification, enzymatic cleavage, and the like, of the administered compound. Accordingly, the invention includes metabolites of compounds, a derivative thereof, or a conjugate thereof, of the invention, including compounds, a derivative thereof, or a conjugate thereof, produced by a process comprising contacting a compound, a derivative thereof, or a conjugate thereof, of this invention with a mammal for a period of time sufficient to yield a metabolic product thereof.

The phrase “pharmaceutically acceptable” indicates that the substance or composition must be compatible chemically and/or toxicologically, with the other ingredients comprising a formulation, and/or the mammal being treated therewith.

The phrase “pharmaceutical composition” refers to a composition comprising a compound or a conjugate of the present invention and a pharmaceutically acceptable carrier.

The term “protecting group” or “protecting moiety” refers to a substituent that is commonly employed to block or protect a particular functionality while reacting other functional groups on the compound, a derivative thereof, or a conjugate thereof. For example, an “amine-protecting group” or an “amino-protecting moiety” is a substituent attached to an amino group that blocks or protects the amino functionality in the compound. Such groups are well known in the art (see for example P. Wuts and T. Greene, 2007, Protective Groups in Organic Synthesis, Chapter 7, J. Wiley & Sons, NJ) and exemplified by carbamates such as methyl and ethyl carbamate, FMOC, substituted ethyl carbamates, carbamates cleaved by 1,6-β-elimination (also termed “self immolative”), ureas, amides, peptides, alkyl and aryl derivatives. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see P. G. M. Wuts & T. W. Greene, Protective Groups in Organic Synthesis, John Wiley & Sons, New York, 2007.

The term “leaving group” refers to an group of charged or uncharged moiety that departs during a substitution or displacement. Such leaving groups are well known in the art and include, but not limited to, halogens, esters, alkoxy, hydroxyl, tosylates, triflates, mesylates, nitriles, azide, carbamate, disulfides, thioesters, thioethers and diazonium compounds.

The term “bifunctional crosslinking agent,” “bifunctional linker” or “crosslinking agents” refers to modifying agents that possess two reactive groups; one of which is capable of reacting with a cell binding agent while the other one reacts with the cytotoxic compound to link the two moieties together. Such bifunctional crosslinkers are well known in the art (see, for example, Isalm and Dent in Bioconjugation chapter 5, p 218-363, Groves Dictionaries Inc. New York, 1999). For example, bifunctional crosslinking agents that enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that introduce maleimido groups or haloacetyl groups on to a cell binding agent are well known in the art (see US Patent Applications 2008/0050310, 20050169933, available from Pierce Biotechnology Inc. P.O. Box 117, Rockland, Ill. 61105, USA) and include, but not limited to, bis-maleimidopolyethyleneglycol (BMPEO), BM(PEO)₂, BM(PEO)₃, N-(β-maleimidopropyloxy)succinimide ester (BMPS), γ-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(p-maleimidophenyl)-butyrate (SMPB), succinimidyl-6-(β-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(γ-maleimidobutryloxy)sulfosuccinimide ester (sulfo-GMBS), N-(ε-maleimidocaproyloxy)sulfosuccimido ester (sulfo-EMCS), N-(κ-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents containing both an amine-reactive N-hydroxysuccinimide group (NHS group) and a carbonyl-reactive hydrazine group can also be used to link the cytotoxic compounds described herein with a cell-binding agent (e.g., antibody). Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present invention. Examples of bifunctional crosslinking agents that can be used include succinimidyl-p-formyl benzoate (SFB) and succinimidyl-p-formylphenoxyacetate (SFPA).

Bifunctional crosslinking agents that enable the linkage of cell binding agent with cytotoxic compounds via disulfide bonds are known in the art and include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. Other bifunctional crosslinking agents that can be used to introduce disulfide groups are known in the art and are disclosed in U.S. Pat. Nos. 6,913,748, 6,716,821 and US Patent Publications 20090274713 and 20100129314, all of which are incorporated herein by reference. Alternatively, crosslinking agents such as 2-iminothiolane, homocysteine thiolactone or S-acetylsuccinic anhydride that introduce thiol groups can also be used.

A “reactive moiety” or “reactive group” as defined herein refers to a chemical moiety that form a covalent bond with another chemical group. For example, a reactive moiety can reactive with certain groups on the cell-binding agent (CBA) to form a covalent bond. In some embodiments, the reactive moiety is an amine reactive group that can form a covalent bond with ε-amine of a lysine residue located on the CBA. In another embodiment, a reactive moiety is an aldehyde reactive group that can form a covalent bond with an aldehyde group located on the CBA. In yet another embodiment, a reactive moiety is a thiol reactive group that can form a covalent bond with the thiol group of a cysteine residue located on the CBA.

A “linker,” “linker moiety,” or “linking group” as defined herein refers to a moiety that connects two groups, such as a cell binding agent and a cytotoxic compound, together. Typically, the linker is substantially inert under conditions for which the two groups it is connecting are linked. A bifunctional crosslinking agent may comprise two reactive groups, one at each ends of a linker moiety, such that one reactive group can be first reacted with the cytotoxic compound to provide a compound bearing the linker moiety and a second reactive group, which can then react with a cell binding agent. Alternatively, one end of the bifunctional crosslinking agent can be first reacted with the cell binding agent to provide a cell binding agent bearing a linker moiety and a second reactive group, which can then react with a cytotoxic compound. The linking moiety may contain a chemical bond that allows for the release of the cytotoxic moiety at a particular site. Suitable chemical bonds are well known in the art and include disulfide bonds, thioether bonds, acid labile bonds, photolabile bonds, peptidase labile bonds and esterase labile bonds (see for example U.S. Pat. Nos. 5,208,020; 5,475,092; 6,441,163; 6,716,821; 6,913,748; 7,276,497; 7,276,499; 7,368,565; 7,388,026 and 7,414,073). Preferred are disulfide bonds, thioether and peptidase labile bonds. Other linkers that can be used in the present invention include non-cleavable linkers, such as those described in are described in detail in U.S. publication number 20050169933, or charged linkers or hydrophilic linkers and are described in US 2009/0274713, US 2010/01293140 and WO 2009/134976, each of which is expressly incorporated herein by reference, each of which is expressly incorporated herein by reference.

In some embodiments, the linking group with a reactive group attached at one end, such as a reactive ester, is selected from the following: —O(CR₂₀R₂₁)ₘ(CR₂₂R₂₃)_{n′}(OCH₂CH₂)ₚ(CR₄₀R₄₁)_{p″}Y″(CR₂₄R₂₅)_{q}(CO)ₜX″, —O(CR₂₀R₂₁)ₘ(CR₂₆═CR₂₇)_{m′}(CR₂₂R₂₃)ₙ(OCH₂CH₂)ₚ(CR₄₀R₄₁)_{p″}Y″(CR₂₄R₂₅)_{q}(CO)ₜX″, —O(CR₂₀R₂₁)ₘ(alkynyl)_{n′}(CR₂₂R₂₃)ₙ(OCH₂CH₂)ₚ(CR₄₀R₄₁)_{p″}Y″(CR₂₄R₂₅)_{q}(CO)ₜX″, —O(CR₂₀R₂₁)ₘ(piperazino)_{t′}(CR₂₂R₂₃)ₙ(OCH₂CH₂)ₚ(CR₄₀R₄₁)_{p″}Y″, (CR₂₄R₂₅)_{q}(CO)ₜX″, —O(CR₂₀R₂₁)ₘ(pyrrolo)_{t′}(CR₂₂R₂₃)ₙ(OCH₂CH₂)ₚ(CR₄₀R₄₁)_{p″}Y″, (CR₂₄R₂₅)_{q}(CO)ₜX″, —O(CR₂₀R₂₁)ₘA″ₘ″(CR₂₂R₂₃)ₙ(OCH₂CH₂)ₚ(CR₄₀R₄₁)_{p″}Y″(CR₂₄R₂₅)_{q}(CO)ₜX″, —S(CR₂₀R₂₁)ₘ(CR₂₂R₂₃)ₙ(OCH₂CH₂)ₚ(CR₄₀R₄₁)_{p″}Y″(CR₂₄R₂₅)_{q}(CO)ₜX″, —S(CR₂₀R₂₁)ₘ(CR₂₆═CR₂₇)_{m′}(CR₂₂R₂₃)ₙ(OCH₂CH₂)ₚ(CR₄₀R₄₁)_{p″}Y″, (CR₂₄R₂₅)_{q}(CO)ₜX″, —S(CR₂₀R₂₁)ₘ(alkynyl)ₘ(CR₂₂R₂₃)ₙ(OCH₂CH₂)ₚ(CR₄₀R₄₁)_{p″}Y″(CR₂₄R₂₅)_{q}(CO)ₜX″, —S(CR₂₀R₂₁)ₘ(piperazino)_{t′}(CR₂₂R₂₃)ₙ(OCH₂CH₂)ₚ(CR₄₀R₄₁)_{p″}Y″(CR₂₄R₂₅)_{q}(CO)ₜX″, —S(CR₂₀R₂₁)ₘ(pyrrolo)_{t′}(CR₂₂R₂₃)ₙ(OCH₂CH₂)ₚ(CR₄₀R₄₁)_{p″}Y″(CR₂₄R₂₅)_{q}(CO)ₜX″, —S(CR₂₀R₂₁)ₘA″_{m″}(CR₂₂R₂₃)ₙ(OCH₂CH₂)ₚ(CR₄₀R₄₁)_{p″}Y″(CR₂₄R₂₅)_{q}(CO)ₜX″, —NR₃₃(C═O)_{p″}(CR₂₀R₂₁)ₘ(CR₂₂R₂₃)ₙ(OCH₂CH₂)ₚ(CR₄₀R₄₁)_{p″}Y″(CR₂₄R₂₅)_{q}(CO)ₜX″, —NR₃₃(C═O)_{p″}(CR₂₀R₂₁)ₘ(CR₂₆═CR₂₇)_{m′}(CR₂₂R₂₃)ₙ(OCH₂CH₂)ₚ(CR₄₀R₄₁)_{p″}Y″(CR₂₄R₂₅)_{q}(CO)ₜX″, —NR₃₃(C═O)_{p″}(CR₂₀R₂₁)ₘ(alkynyl)_{n′}(CR₂₂R₂₃)ₙ(OCH₂CH₂)ₚ(CR₄₀R₄₁)_{p″}Y″, (CR₂₄R₂₅)_{q}—(CO)ₜX″, —NR₃₃(C═O)_{p″}(CR₂₀R₂₁)ₘ(piperazino)ₜ(CR₂₂R₂₃)ₙ(OCH₂CH₂)ₚ(CR₄₀R₄₁)_{p″}Y″(CR₂₄R₂₅)_{q}(CO)ₜX″, —NR₃₃(C═O)_{p″}(CR₂₀R₂₁)ₘ(pyrrolo)ₜ(CR₂₂R₂₃)ₙ(OCH₂CH₂)ₚ(CR₄₀R₄₁)_{p″}Y″(CR₂₄R₂₅)_{q}—(CO)ₜX″, —NR₃₃(C═O)_{p″}(CR₂₀R₂₁)ₘA″_{m″}(CR₂₂R₂₃)ₙ(OCH₂CH₂)ₚ(CR₄₀R₄₁)_{p″}Y″(CR₂₄R₂₅)_{q} (CO)ₜX″, —(CR₂₀R₂₁)ₘ(CR₂₂R₂₃)ₙ(OCH₂CH₂)ₚ(CR₄₀R₄₁)_{p″}Y″(CR₂₄R₂₅)_{q}(CO)ₜX″, —(CR₂₀R₂₁)ₘ(CR₂₆═CR₂₇)_{m′}(CR₂₂R₂₃)ₙ(OCH₂CH₂)ₚ(CR₄₀R₄₁)_{p″}Y″(CR₂₄R₂₅)_{q}(CO)ₜX, —(CR₂₀R₂₁)ₘ(alkynyl)_{n′}(CR₂₂R₂₃)ₙ(OCH₂CH₂)ₚ(CR₄₀R₄₁)_{p″}Y″(CR₂₄R₂₅)_{q}(CO)ₜX″, —(CR₂₀R₂₁₁)ₘ(piperazino)_{t′}(CR₂₂R₂₃)ₙ(OCH₂CH₂)ₚ(CR₄₀R₄₁)_{p″}Y″(CR₂₄R₂₅)_{q}(CO)ₜX″, —(CR₂₀R₂₁)ₘA″_{m″}(CR₂₂R₂₃)ₙ(OCH₂CH₂)ₚ(CR₄₀R₄₁)_{p″}Y″(CR₂₄R₂₅)_{q}(CO)ₜX, —(CR₂₀R₂₁)ₘ(CR₂₉═N—NR₃₀)_{n″}(CR₂₂R₂₃)ₙ(OCH₂CH₂)ₚ(CR₄₀R₄₁)_{p″}Y″(CR₂₄R₂₅)_{q}(CO)ₜX″, —(CR₂₀R₂₁)ₘ(CR₂₉═N—NR₃₀)_{n″}(CR₂₆═CR₂₇)_{m′}(CR₂₂R₂₃)ₙ(OCH₂CH₂)ₚ(CR₄₀R₄₁)_{p″}Y″(CR₂₄R₂₅)_{q}(CO)ₜX″, —(CR₂₀R₂₁)ₘ(CR₂₉═N—NR₃₀)_{n″}(alkynyl)_{n′}(CR₂₂R₂₃)ₙ(OCH₂CH₂)ₚ(CR₄₀R₄₁)_{p″}Y″(CR₂₄R₂₅)_{q}⁻(CO)ₜX″, —(CR₂₀R₂₁)ₘ(CR₂₉═N—NR₃₀)_{n″}A″_{m″}(CR₂₂R₂₃)ₙ(OCH₂CH₂)ₚ(CR₄₀R₄₁)_{p″}Y″(CR₂₄R₂₅)_{q} (CO)ₜX″,

wherein:

m, n, p, q, m′, n′, t′ are integer from 1 to 10, or are optionally 0;

t, m″, n″, and p″ are 0 or 1;

X″ is selected from OR₃₆, SR₃₇, NR₃₈R₃₉, wherein R₃₆, R₃₇, R₃₈, R₃₉ are H, or linear, branched or cyclic alkyl, alkenyl or alkynyl having from 1 to 20 carbon atoms and, or, a polyethylene glycol unit —(OCH₂CH₂)ₙ, R₃₇, optionally, is a thiol protecting group when t=1, COX″ forms a reactive ester selected from N-hydroxysuccinimide esters, N-hydroxyphthalimide esters, N-hydroxy sulfo-succinimide esters, para-nitrophenyl esters, dinitrophenyl esters, pentafluorophenyl esters and their derivatives, wherein said derivatives facilitate amide bond formation;

Y″ is absent or is selected from O, S, S—S or NR₃₂, wherein R₃₂ has the same definition as given above for R; or

when Y″ is not S—S and t=0, X″ is selected from a maleimido group, a haloacetyl group or SR₃₇, wherein R₃₇ has the same definition as above;

A″ is an amino acid residue or a polypeptide containing between 2 to 20 amino acid residues;

R₂₀, R₂₁, R₂₂, R₂₃, R₂₄, R₂₅, R₂₆, and R₂₇ are the same or different, and are —H or a linear or branched alkyl having from 1 to 5 carbon atoms;

R₂₉ and R₃₀ are the same or different, and are —H or alkyl from 1 to 5 carbon atoms;

R₃₃ is —H or linear, branched or cyclic alkyl, alkenyl or alkynyl having from 1 to 12 carbon atoms, a polyethylene glycol unit R—(OCH₂CH₂)ₙ—, or R₃₃ is —COR₃₄, —CSR₃₄, —SOR₃₄, or —SO₂R₃₄, wherein R₃₄ is H or linear, branched or cyclic alkyl, alkenyl or alkynyl having from 1 to 20 carbon atoms or, a polyethylene glycol unit —(OCH₂CH₂)ₙ; and

one of R₄₀ and R₄₁ is optionally a negatively or positively charged functional group and the other is H or alkyl, alkenyl, alkynyl having 1 to 4 carbon atoms.

Any of the above linking groups may be present in any of the compounds, drug-linker compounds, or conjugates of the invention, including replacing the linking groups of any of the formulas described herein.

The term “amino acid” refers to naturally occurring amino acids or non-naturally occurring amino acid. In some embodiments, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl, or R^{aa} and the N-terminal nitrogen atom can together form a heterocyclic ring (e.g., as in proline). The term “amino acid residue” refers to the corresponding residue when one hydrogen atom is removed from the amine and/or carboxy end of the amino acid, such as —NH—C(R^{aa}R^{aa′})—C(═O)O—.

The term “cation” refers to an ion with positive charge. The cation can be monovalent (e.g., Na⁺, K⁺, etc.), bi-valent (e.g., Ca²⁺, Mg²⁺, etc.) or multi-valent (e.g., Al³⁺ etc.). In some embodiments, the cation is monovalent.

The term “therapeutically effective amount” means that amount of active compound or conjugate that elicits the desired biological response in a subject. Such response includes alleviation of the symptoms of the disease or disorder being treated, prevention, inhibition or a delay in the recurrence of symptom of the disease or of the disease itself, an increase in the longevity of the subject compared with the absence of the treatment, or prevention, inhibition or delay in the progression of symptom of the disease or of the disease itself. Determination of the effective amount is well within the capability of those skilled in the art, especially in light of the detailed disclosure provided herein. Toxicity and therapeutic efficacy of compound I can be determined by standard pharmaceutical procedures in cell cultures and in experimental animals. The effective amount of compound or conjugate of the present invention or other therapeutic agent to be administered to a subject will depend on the stage, category and status of the multiple myeloma and characteristics of the subject, such as general health, age, sex, body weight and drug tolerance. The effective amount of compound or conjugate of the present invention or other therapeutic agent to be administered will also depend on administration route and dosage form. Dosage amount and interval can be adjusted individually to provide plasma levels of the active compound that are sufficient to maintain desired therapeutic effects.

Cytotoxic Compounds

In a first aspect, the present invention is directed to cytotoxic compounds described herein.

In some embodiments, the cytotoxic compound is represented by structural formula (I):

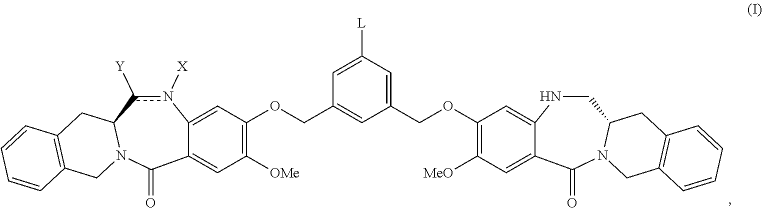

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;

L is represented by the following formula:

—NR₅—P—C(═O)—W-J (L1);

—NR₅—P—C(═O)—W—S—Z^{s} (L2);

—N(R^{e′})—W—S—Z^{s} (L3);

—N(R^{e})—C(═O)—W—S—Z^{s} (L4); or

—N(R^{e′})—W-J (L5);

R₅, for each occurrence, is independently H or a (C₁-C₃)alkyl;

W is a spacer unit;

J is a reactive moiety capable of forming a covalent bond with a cell-binding agent;

R^{e} is H or a (C₁-C₃)alkyl;

R^{e′} is —(CH₂—CH₂—O)ₙ—R^{k};

n is an integer from 2 to 6;

R^{k} is H or Me;

Z^{s} is H, —SR^{d}, —C(═O)R^{d1} or a bifunctional linker having a reactive moiety capable of forming a covalent bond with a cell-binding agent;

R^{d} is a (C₁-C₆)alkyl or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and

R^{d1} is a (C₁-C₆)alkyl.

In a more specific embodiment, W is a linear, branched or cyclic alkyl, alkenyl, alkynyl, an aryl, a heteroaryl, or a heterocycloalkyl.

In another more specific embodiment, J is —COOR^{c} or —C(═O)E, wherein R^{c} is H or a (C₁-C₃)alkyl; and —C(═O)E represents a reactive ester.

In a first embodiment, the cytotoxic compound of the present invention has an amine-reactive group that can form a covalent bond with the ε-amino group of one or more lysine residues located on the cell-binding agents described herein.

In a 1^{st} specific embodiment, the cytotoxic compound is represented by the following formula:

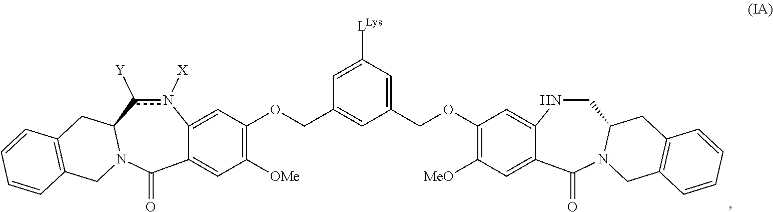

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;

L^{Lys} is represented by the following formula:

—NR₅—P—C(═O)—(CR^{a}R^{b})ₘ-J^{Lys} (L1);

—NR₅—P—C(═O)—(CR^{a}R^{b})ₘ—S—Z^{s} (L2);

—N(R^{e})—C(═O)—R^{x1}—S—Z^{s} (L3);

—N(R^{e′})—R^{x2}—S—Z^{s} (L4);

—N(R^{e′})—R^{x3}-J^{Lys} (L5);

R₅ is —H or a (C₁-C₃)alkyl;

P is an amino acid residue or a peptide containing between 2 to 20 amino acid residues;

Rₐ and R_{b}, for each occurrence, are each independently —H, (C₁-C₃)alkyl, or a charged substituent or an ionizable group Q;

m is an integer from 1 to 6;

R^{x1} and R^{x2} are independently (C₁-C₆)alkyl;

R^{x3} is a (C₁-C₆)alkyl;

R^{e} is —H or a (C₁-C₆)alkyl;

R^{e′} is —(CH₂—CH₂—O)ₙ—R^{k};

n is an integer from 2 to 6;

R^{k} is —H or -Me;

J^{Lys} is —COOR^{c} or —C(═O)E, wherein R^{c} is H or a (C₁-C₃)alkyl; and —C(═O)E represents a reactive ester;

Z^{s} is H, —SR^{d}, —C(═O)R^{d1} or is selected from any one of the following formulae:

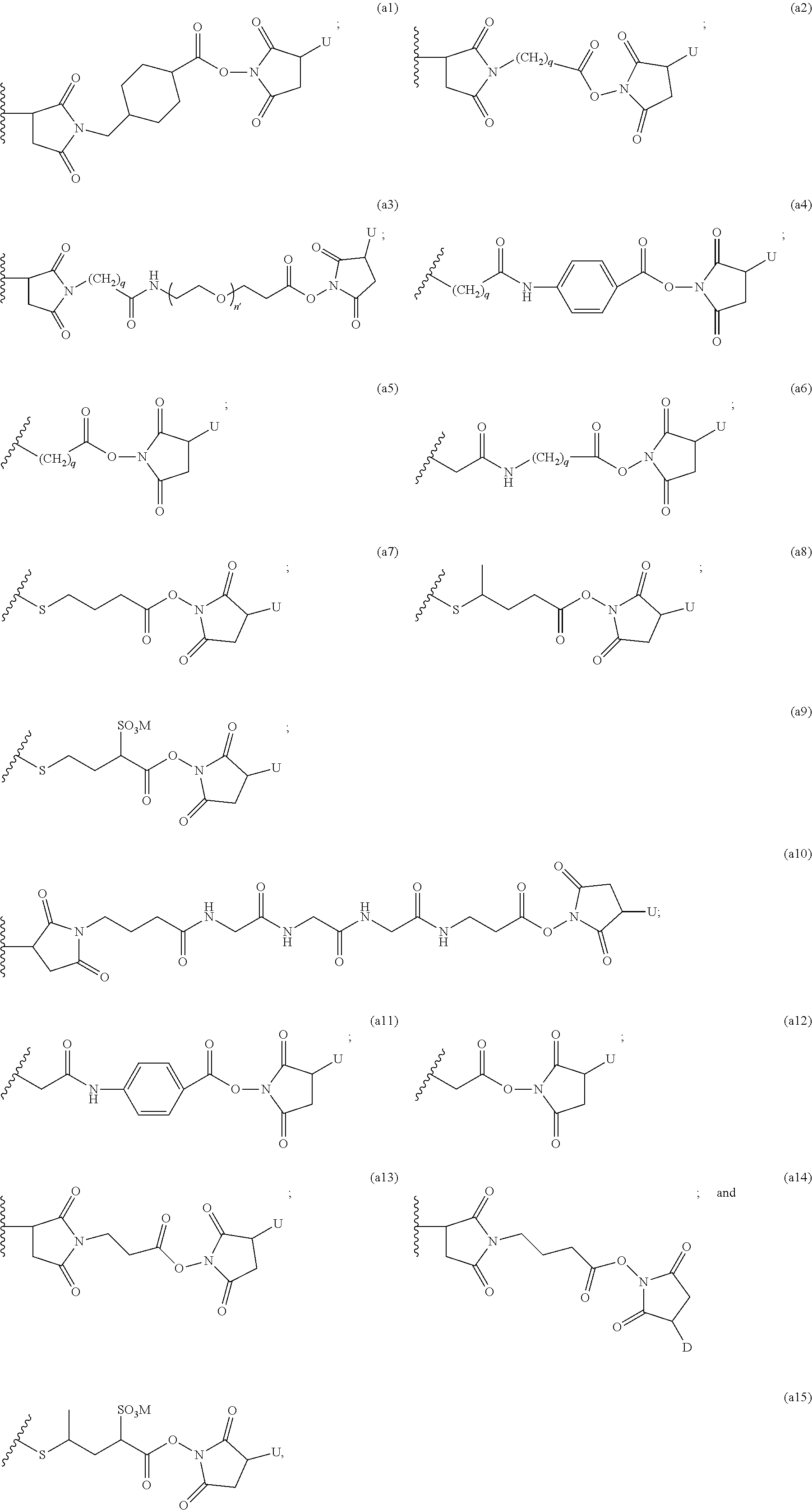

q is an integer from 1 to 5;

n′ is an integer from 2 to 6;

U is H or SO₃M;

M is H or a pharmaceutically acceptable cation;

R^{d} is a (C₁-C₆)alkyl or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and

R^{d1} is a (C₁-C₆)alkyl.

In a 2^{nd} specific embodiment, L^{Lys} is represented by formula (L1) or (L2); and the remaining variables are as described above in the 1^{st} specific embodiment.

In a 3^{rd} specific embodiment, L^{Lys} is represented by formula (L5); and the remaining variables are as described above in the 1^{st} specific embodiment. More specifically, R^{x3} is a (C₂-C₄)alkyl.

In a 4^{th} specific embodiment, for formulae (L1) and (L2), Rₐ and R_{b} are both H; R₅ is H or Me, and the remaining variables are as described above in the 1^{st} specific embodiment.

In a 5^{th} specific embodiment, for formulae (L1) and (L2), P is a peptide containing 2 to 5 amino acid residues; and the remaining variables are described above in the 1^{st}, 2^{nd} or 4^{th} specific embodiment. In a more specific embodiment, P is selected from the group consisting of Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), β-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. More specifically, P is Gly-Gly-Gly, Ala-Val, Ala-Ala, Ala-D-Ala, D-Ala-Ala, or D-Ala-D-Ala.

As used herein, the peptide represented by P or P′ can be connected to the rest of the molecules in both directions. For example, a dipeptide X₁-X₂ includes X₁-X₂ and X₂—X₁. Similarly, a tripeptide X₁-X₂-X₃ includes X₁-X₂-X₃ and X₃-X₂-X₁ and a tetrapeptide X₁-X₂-X₃-X₄ includes X₁-X₂-X₃-X₄ and X₄-X₂-X₃-X₁. X₁, X₂, X₃ and X₄ represents an amino acid.

In a 6^{th} specific embodiment, Q is —SO₃M; and the remaining variables are as described above in the 1^{st}, 2^{nd}, 4^{th} or 5^{th} specific embodiment or any more specific embodiments described therein.

In a 7^{th} specific embodiment, for formulae (L1) and (L5), J^{Lys} is a reactive ester selected from the group consisting of N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4 sulfo-2,3,5,6-tetrafluorophenyl) ester, and pentafluorophenyl ester; and the remaining variables are as described in the 1^{st}, 2^{nd}, 3^{rd}, 4^{th}, 5^{th} or 6^{th} specific embodiment or any more specific embodiments described therein. More specifically, J^{Lys} is N-hydroxysuccinimide ester.

In a 8^{th} specific embodiment, for formulae (L2), (L3) and (L4), Z^{s} is H or —SR^{d}, wherein R^{d} is a (C₁-C₃)alkyl, pyridyl or nitropyridyl (e.g., 4-nitropyridyl); and the remaining variables are as described in the 1^{st}, 2^{nd}, 4^{th}, 5^{th} or 6^{th} specific embodiment or any more specific embodiments described therein.

In a 9^{th} specific embodiment, for formulae (L2), (L3) and (L4), Z^{s} is selected from any one of the following formulae:

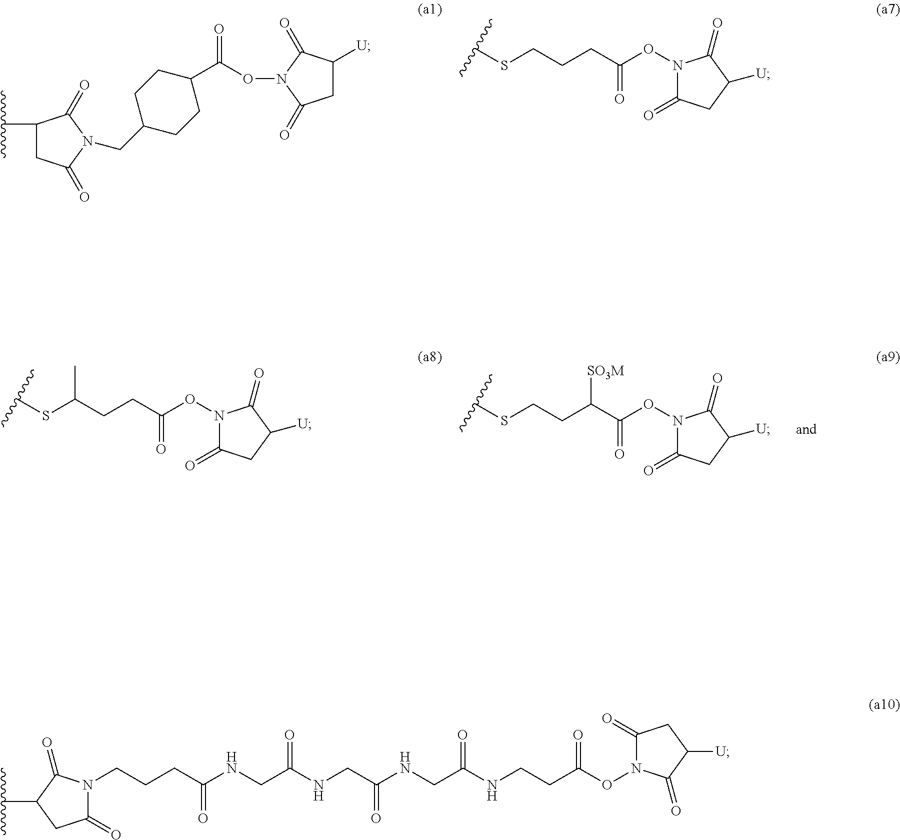

and the remaining variables are as described in the 1^{st}, 2^{nd}, 4^{th}, 5^{th} or 6^{th} specific embodiment or any more specific embodiments described therein.

In a 10^{th} specific embodiment, for cytotoxic compounds of formula (IA), the double line [symbol] between N and C represents a double bond, X is absent and Y is —H; and the remaining variables are as described in the 1^{st}, 2^{nd}, 3^{rd}, 4^{th}, 5^{th}, 6^{th}, 7^{th}, 8^{th} or 9^{th} specific embodiment or any more specific embodiments described therein.

In a 11^{th} specific embodiment, for cytotoxic compounds of formula (IA), the double line [symbol] between N and C represents a single bond, X is H and Y is —SO₃M; and [symbol] the remaining variables are as described in the 1^{st}, 2^{nd}, 3^{rd}, 4^{th}, 5^{th}, 6^{th}, 7^{th}, 8^{th} or 9^{th} specific embodiment or any more specific embodiments described therein.

In a 12^{th} specific embodiment, for cytotoxic compounds of formula (IA), the double line [symbol] between N and C represents a single bond or a double bond, provided that when it is a double bond, X is absent and Y is —H; and when it is a single bond, X is —H, and Y is —SO₃M;

M is H, Na⁺ or K⁺;

L^{Lys} is represented by the following formula:

—NR₅—P—C(═O)—(CR^{a}R^{b})ₘ-J^{Lys} (L1);

wherein:
R^{a} and R^{b} are both —H;
m is 3 to 5;
P is Ala-Ala, Ala-D-Ala, D-Ala-Ala, or D-Ala-D-Ala;
R₅ is H or Me; and
J^{Lys} is N-hydroxysuccinimide ester or N-hydroxy sulfosuccinimide ester.

In a 13^{th} specific embodiment, for cytotoxic compounds of formula (IA), the double line [symbol] between N and C represents a single bond or a double bond, provided that when it is a double bond, X is absent and Y is —H; and when it is a single bond, X is —H, and Y is —SO₃M;

M is H, N⁺ or K⁺;

L^{Lys} is represented by the following formula:

—NR₅—P—C(═O)—(CR^{a}R^{b})ₘ—S—Z^{s} (L2),

wherein:
(CR^{a}R^{b})ₘ— is —(CH₂)ₚ—(CR^{f}R^{g})—, wherein R^{f} and R^{g} are each independently —H or -Me; and p is 0, 1, 2 or 3;
P is Ala-Ala, Ala-D-Ala, D-Ala-Ala, or D-Ala-D-Ala;
R is H or Me;
Z^{s} is H, —SR^{d} or is represented by formula (a1), (a7), (a8), (a9) or (a10); and
R^{d} is a (C₁-C₃)alkyl, pyridyl or nitropyridyl (e.g., 4-nitropyridyl).

In a 14^{th} specific embodiment, for cytotoxic compounds of formula (IA), the double line [symbol] between N and C represents a single bond or a double bond, provided that when it is a double bond, X is absent and Y is —H; and when it is a single bond, X is —H, and Y is —SO₃M;

M is H, N⁺ or K⁺;

L^{Lys} is represented by the following formula:

—N(R^{e})—C(═O)—R^{x1}—S—Z^{s} (L3);

wherein:
R^{e} is H or Me;
R^{x1} is —(CH₂)ₚ—(CR^{f}R^{g})—, wherein R^{f} and R^{g} are each independently —H or -Me; and p is 0, 1, 2 or 3;
Z^{s} is H, —SR^{d} or is represented by formula (a1), (a7), (a8), (a9) or (a10); and
R^{d} is a (C₁-C₃)alkyl, pyridyl or nitropyridyl (e.g., 4-nitropyridyl).

In a 15^{th} specific embodiment, for cytotoxic compounds of formula (IA), the double line [symbol] between N and C represents a single bond or a double bond, provided that when it is a double bond, X is absent and Y is —H; and when it is a single bond, X is —H, and Y is —SO₃M;

M is H, Na⁺ or K⁺;

L^{Lys} is represented by the following formula:

—N(R^{e′})—R^{x2}—S—Z^{s} (L4);

wherein:
R^{x2} is —(CH₂)ₚ—(CR^{f}R^{g})—, wherein R^{f} and R^{g} are each independently —H or -Me; and p is 0, 1, 2 or 3;
R^{e′} is —(CH₂—CH₂—O)ₙ—R^{k};
R^{k} is Me;
Z^{s} is H, —SR^{d} or is represented by formula (a1), (a7), (a8), (a9) or (a10); and R^{d} is a (C₁-C₃)alkyl, pyridyl or nitropyridyl (e.g., 4-nitropyridyl).

In a 16^{th} specific embodiment, for cytotoxic compounds of formula (IA), the double line [symbol] between N and C represents a single bond or a double bond, provided that when it is a double bond, X is absent and Y is —H; and when it is a single bond, X is —H, and Y is —SO₃M;

M is H, N⁺ or K⁺;

L^{Lys} is represented by the following formula:

—N(R^{e′})—R³-J^{Lys} (L5);

wherein:
R^{e′} is —(CH₂—CH₂—O)ₙ—R^{k};
R^{k} is Me;
R^{x3} is —(CR^{a}R^{b})ₘ—
R^{a} and R^{b} are both —H;
m is 3 to 5; and
J^{Lys} is N-hydroxysuccinimide ester or N-hydroxy sulfosuccinimide ester.

In a 17^{th} specific embodiment, the cytotoxic compounds of the first embodiment is represented by the following formula:

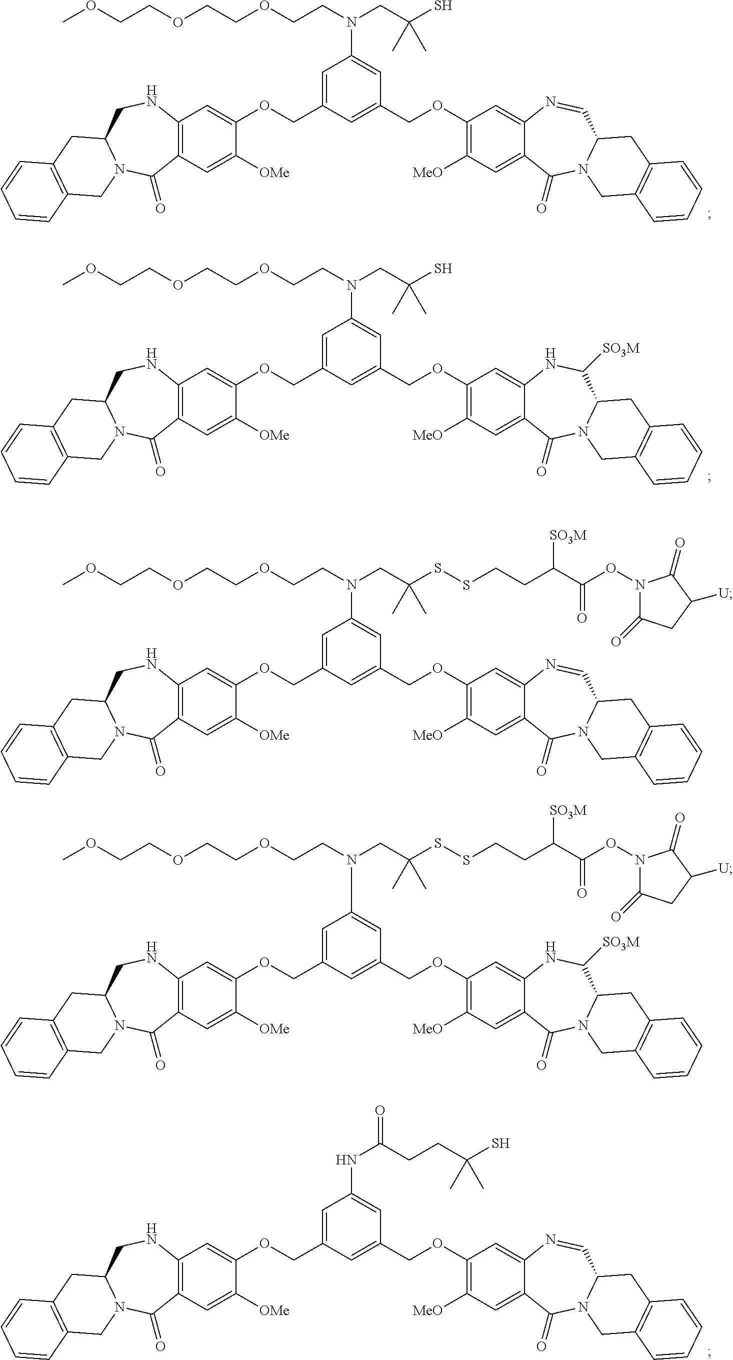

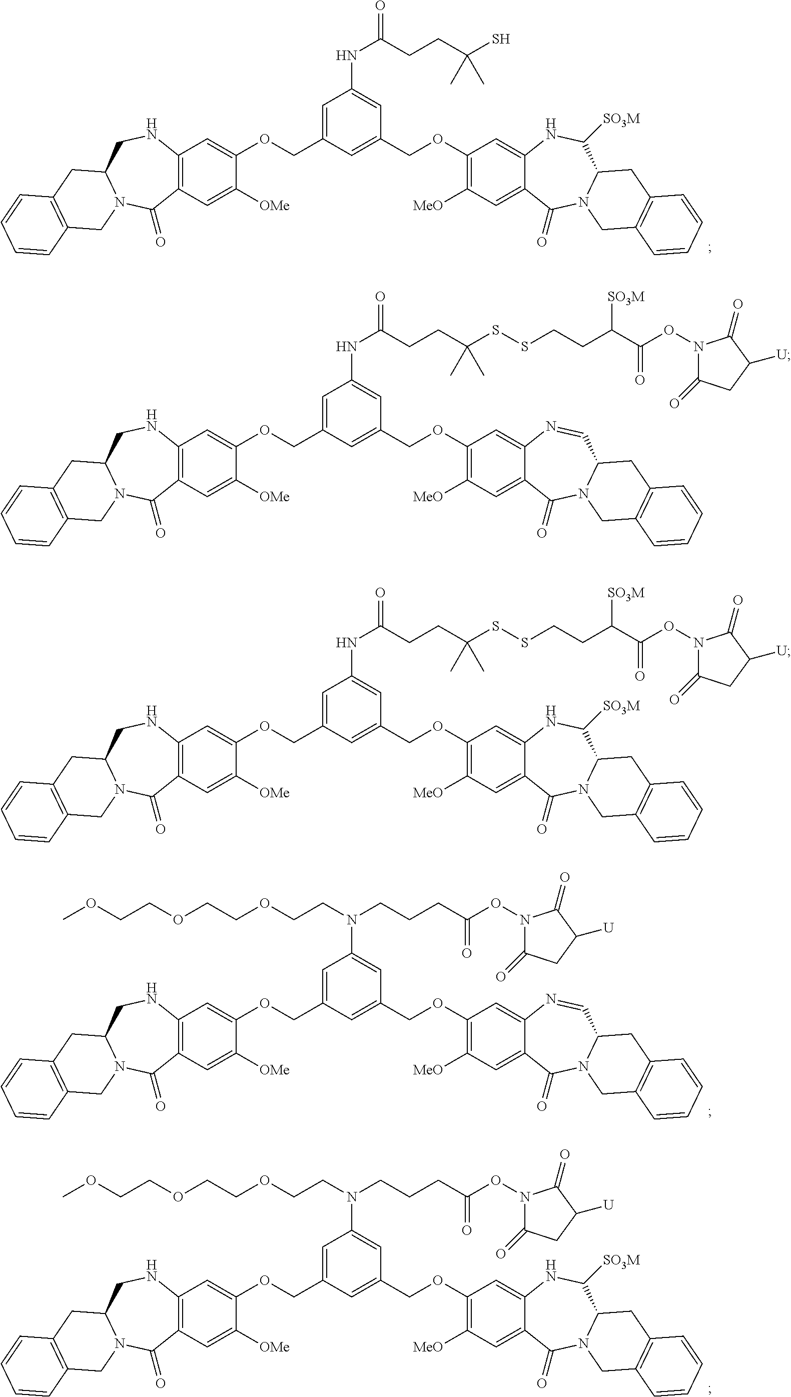

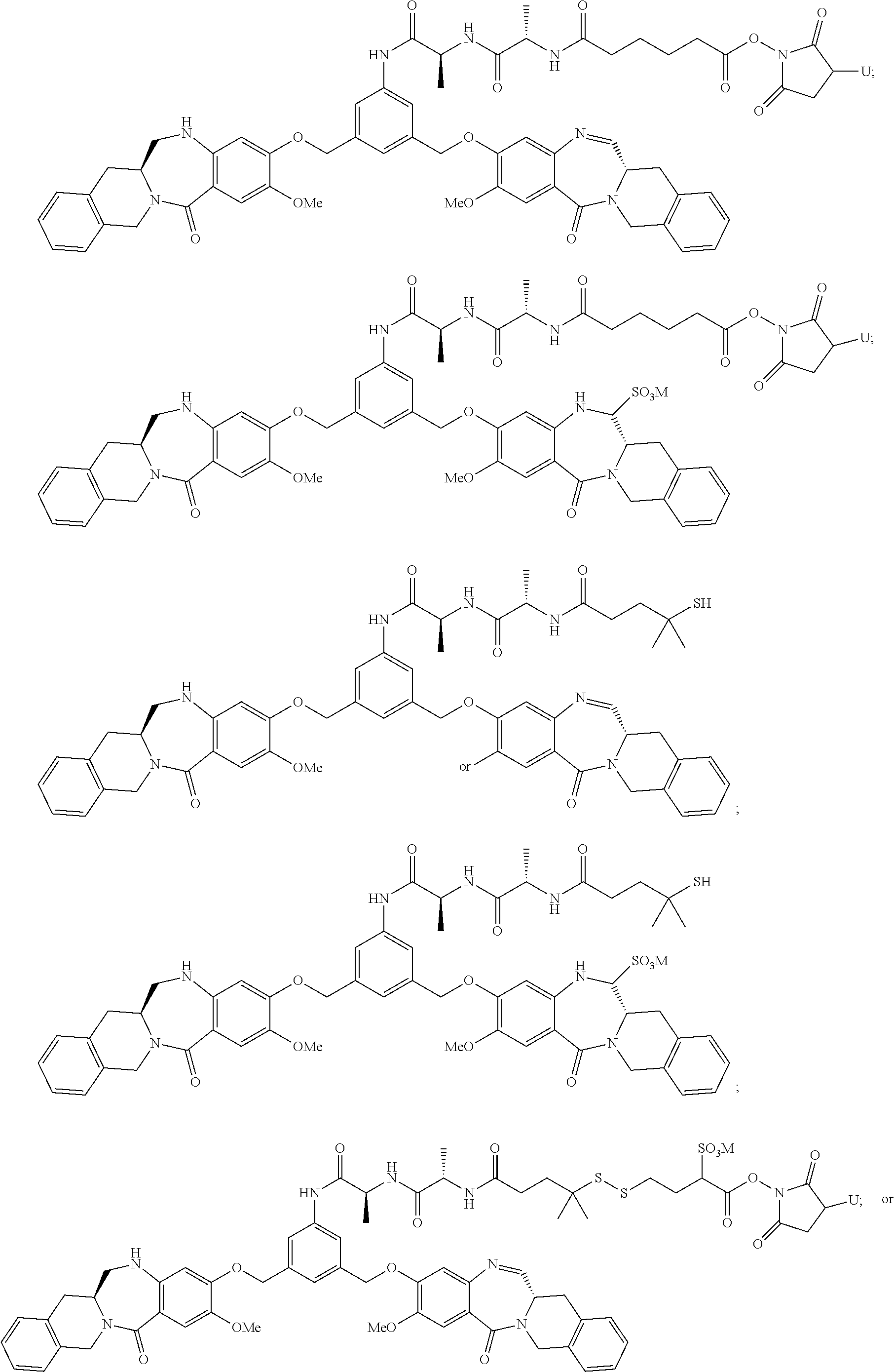

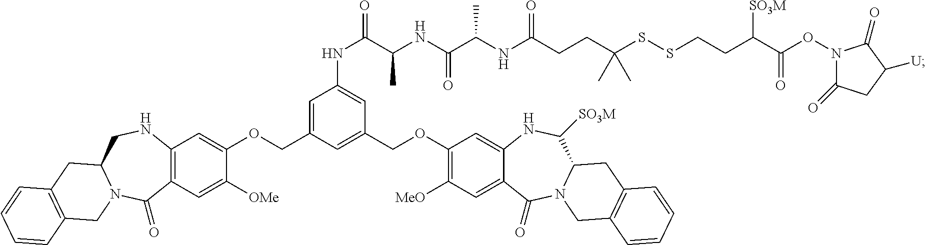

or a pharmaceutically acceptable salt thereof, wherein U is H or SO₃M; and M is H, Na⁺ or K⁺.

In a second embodiment, the cytotoxic compound of the present invention has an aldehyde reactive group that can form a covalent bond with one or more aldehyde groups located on the oxidized cell-binding agent described herein.

In a 1^{st} specific embodiment, the cytotoxic compound is represented by the following formula:

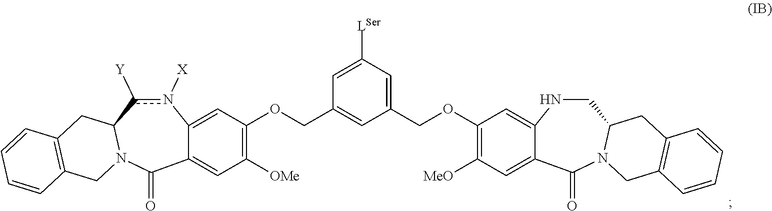

or a pharmaceutically acceptable salt thereof, wherein L^{Ser}:

—NR₅—P—C(═O)—(CR^{a}R^{b})ᵣ—Z_{d1}—(CR^{a}R^{b})_{r′}-J^{Ser} (S1); or

—N(R^{e′})—R^{x3}—C(═O)-L-J^{ser} (S2);

—N(R^{e})—C(═O)—R^{x1}—S-L₁-J^{Ser} (S3)

—N(R^{e′})—R^{x2}—S-L₁-J^{ser} (S4);

wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃M, and M is H⁺ or a cation;

R₅ is —H or a (C₁-C₃)alkyl;

P is an amino acid residue or a peptide containing 2 to 20 amino acid residues;

Z_{d1} is absent, —C(═O)—NR₉—, or —NR₉—C(═O)—;

R₉ is —H or a (C₁-C₃)alkyl;

Rₐ and R_{b}, for each occurrence, are independently —H, (C₁-C₃)alkyl, or a charged substituent or an ionizable group Q;

r and r′ are independently an integer from 1 to 6;

R^{e′} is —(CH₂—CH₂—O)ₙ—R^{k};

n is an integer from 2 to 6;

R^{k} is —H or -Me;

R^{x3} is a (C₁-C₆)alkyl;

L is —NR₉—(CRₐR^{b})_{r″} or absent;

r″ is an integer from 0 to 6;

R^{x1} is a (C₁-C₆)alkyl;

R^{x2} is a (C₁-C₆)alkyl;

L₁ is represented by the following formula:

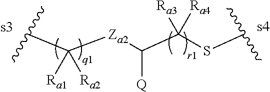

wherein:

s3 is the site covalently linked to the group J^{Ser};

s4 is the site covalently linked to the —S— group on Cy^{Ser}

Zₐ₂ is absent, —C(═O)—NR₉—, or —NR₉—C(═O)—;

Q is H, a charged substituent or an ionizable group;

Rₐ₁, Rₐ₂, Rₐ₃, Rₐ₄, for each occurrence, are independently H or (C₁-C₃)alkyl; and

q1 and r1 are each independently an integer from 0 to 10, provided that q1 and r1 are not both 0; and

J^{Ser} is an aldehyde reactive group.

In some embodiments, J^{Ser} is

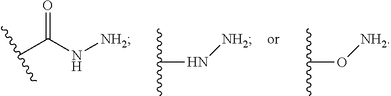

In a 2^{nd} specific embodiment, L^{ser} is represented by formula (S1); and the remaining variables are as described above in the 1^{st} specific embodiment.

In a 3^{rd} specific embodiment, L^{ser} is represented by formula (S2); and the remaining variables are as described above in the 1^{st} specific embodiment. More specifically, R^{x3} is a (C₂-C₄)alkyl.

In a 4^{th} specific embodiment, for formula (S1), Rₐ and R_{b} are both H, and R₅ and R₉ are both H or Me; and the remaining variables are as described above in the 1^{st} or 2^{nd} specific embodiment.

In a 5^{th} specific embodiment, for formula (S1), P is a peptide containing 2 to 5 amino acid residues; and the remaining variables are as described above in the 1^{st}, 2^{nd} or 4^{th} specific embodiment. In a more specific embodiment, P is selected from the group consisting of Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), β-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. Even more specifically, P is Gly-Gly-Gly, Ala-Val, Ala-Ala, Ala-D-Ala, D-Ala-Ala, or D-Ala-D-Ala.

In a 6^{th} specific embodiment, for formula (S1), Q is —SO₃M; and the remaining variables are as described above in the 1^{st}, 2^{nd}, 4^{th} or 5^{th} specific embodiment.

In a 7^{th} specific embodiment, the cytotoxic compound of the second embodiment is represented by the following formula:

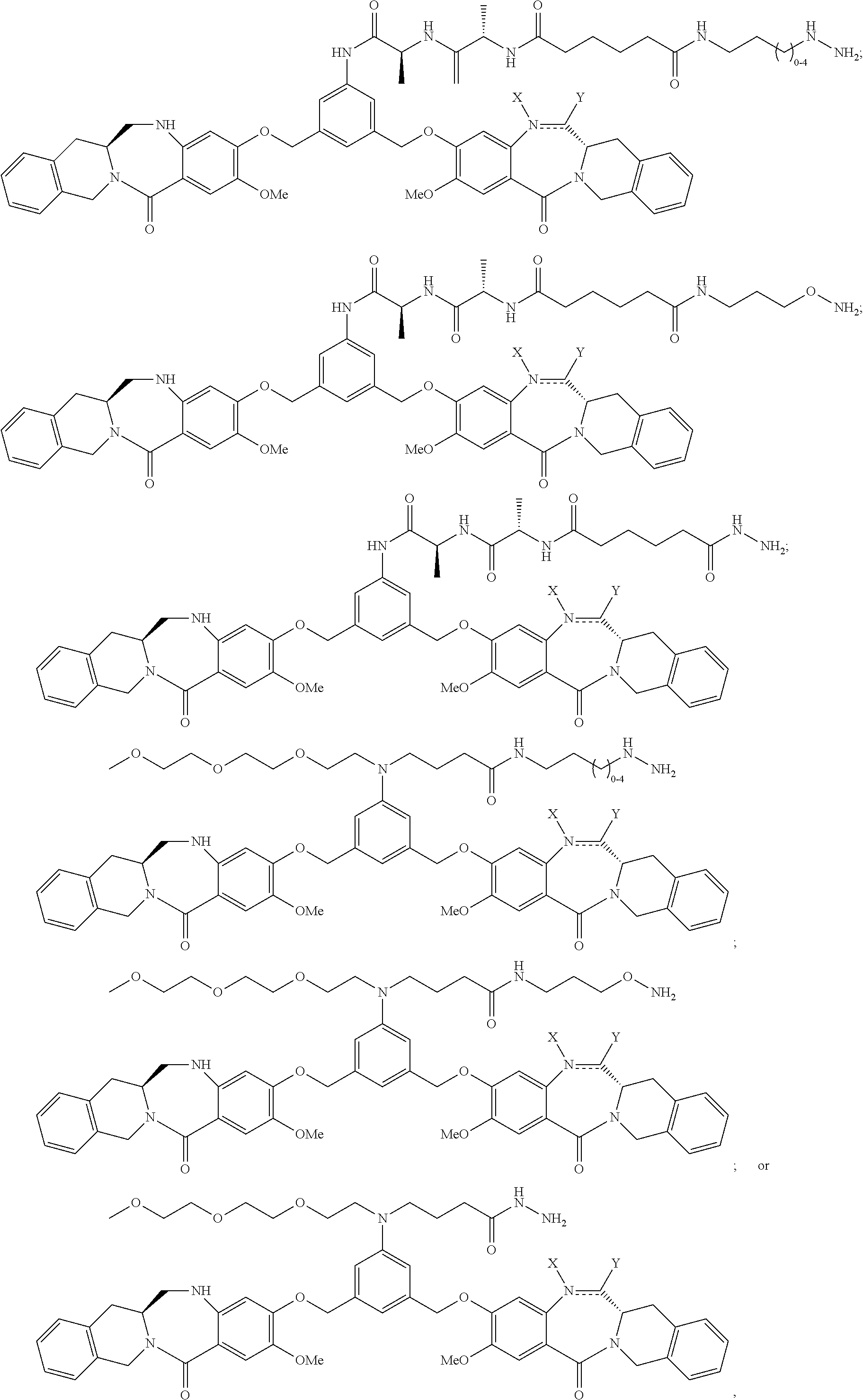

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃M. In a more specific embodiment, the double line [symbol] between N and C represents a double bond, X is absent and Y is —H. In another more specific embodiment, the double line [symbol] between N and C represents a single bond, X is —H and Y is —SO₃M.

In an 8^{th} specific embodiment, L^{Ser} is represented by formula (S3) or (S4), and the remaining variables as described above in the 1^{st} specific embodiment.

In a more specific embodiment, Zₐ₂ is absent; q1 and r1 are each independent an integer from 0 to 3, provided that q1 and r1 are not both 0; and the remaining variables are as described above in the 8^{th} specific embodiments. Even more specifically, Rₐ₁, Rₐ₂, Rₐ₃, Rₐ₄ are all —H.

In another more specific embodiment, Zₐ₂ is —C(═O)—NH—, or —NH₉—C(═O)—; q1 and r1 are each independently an integer from 1 to 6; and the remaining variables are as described above in the 8^{th} specific embodiments. Even more specifically, Rₐ₁, Rₐ₂, Rₐ₃, Rₐ₄ are all —H.

In a 9^{th} specific embodiment, L^{Ser} is represented by formula (S3); and the remaining variables are as described above in the 8^{th} specific embodiment or any more specific embodiments described therein.

In a 10^{th} specific embodiment, L^{Ser} is represented by formula (S4); and the remaining variables are as described above in the 8^{th} specific embodiment or any more specific embodiments described therein.

In an 11^{th} specific embodiment, for formulae (S3) and (S4), -L₁- is represented by the following formula:

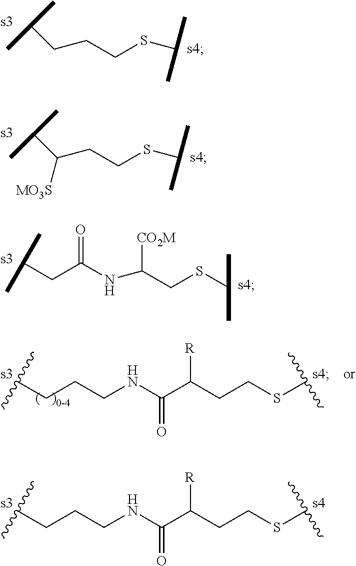

or a pharmaceutically acceptable salt thereof, wherein R is H or —SO₃M; and the remaining variables are as described above in the 8^{th}, 9^{th} or 10^{th} specific embodiment or any more specific embodiments described therein.

In a 12^{th} specific embodiment, for formulae (S3) and (S4), R^{e} is H or Me; and R^{x1} is —(CH₂)ₚ—(CR^{f}R^{g})—, and R^{x2} is —(CH₂)ₚ—(CR^{f}R^{g})—, wherein R^{f} and R^{g} are each independently —H or a (C₁-C₄)alkyl; and p is 0, 1, 2 or 3. More specifically, R^{f} and R^{g} are the same or different, and are selected from —H and -Me.

In a 13^{th} specific embodiment, the cytotoxic compound of the second embodiment is represented by the following formula:

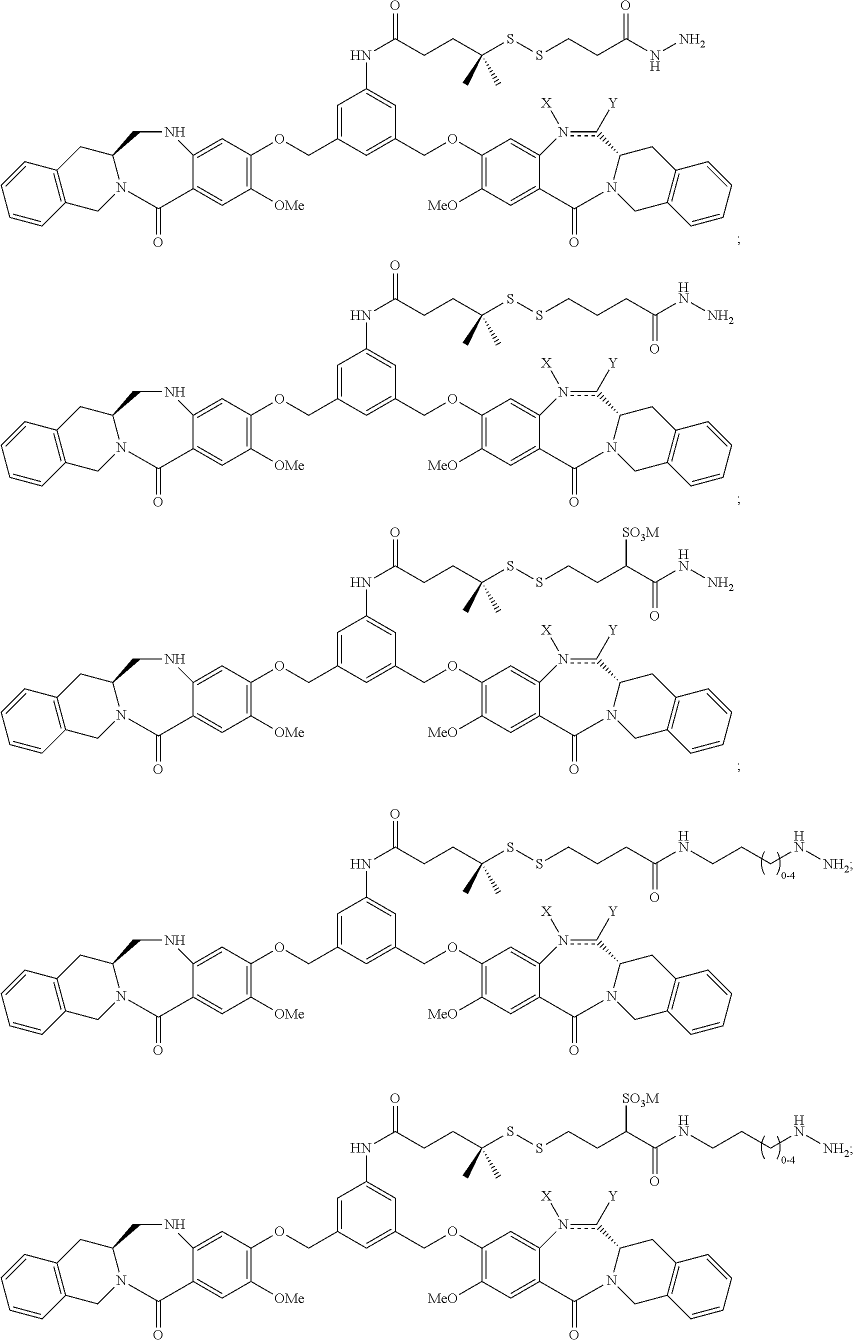

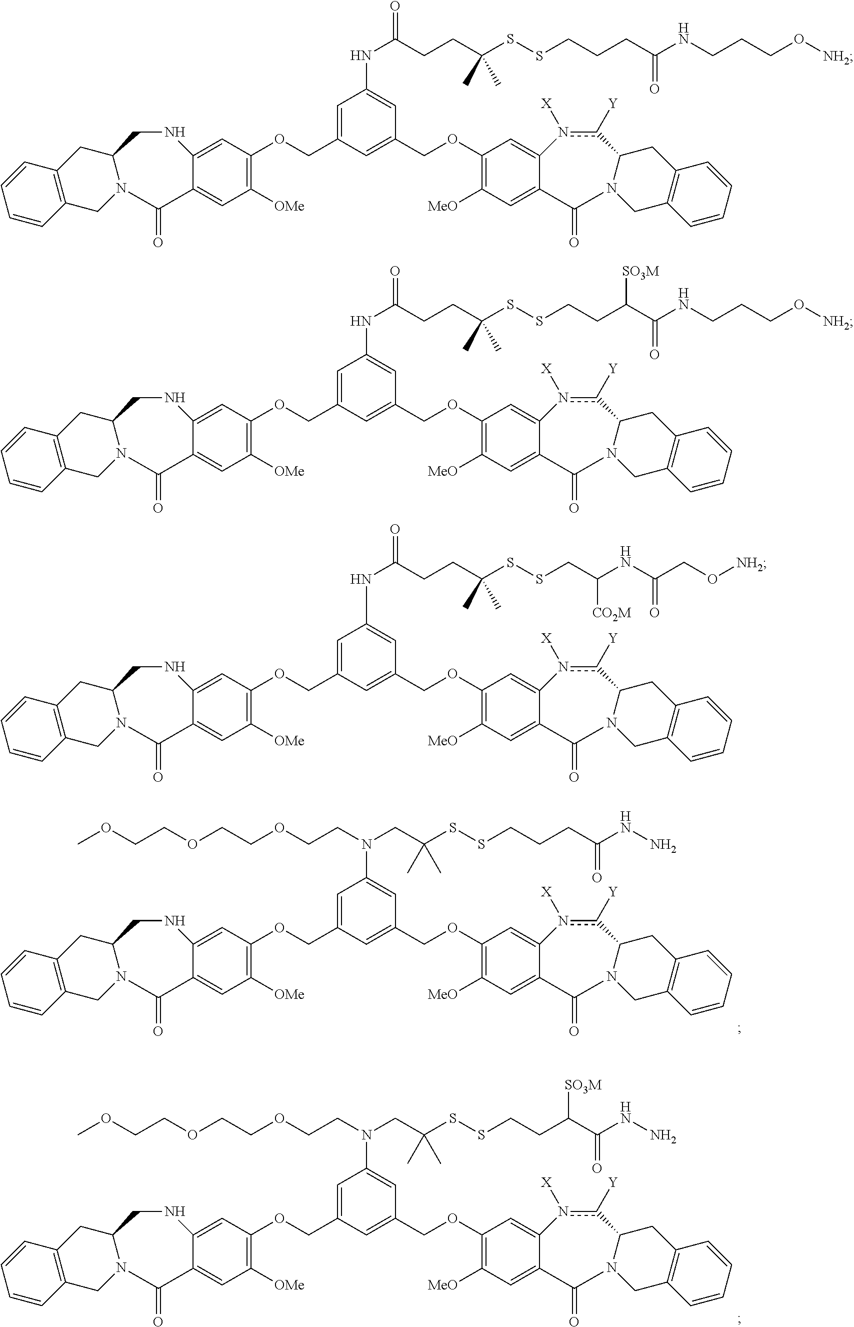

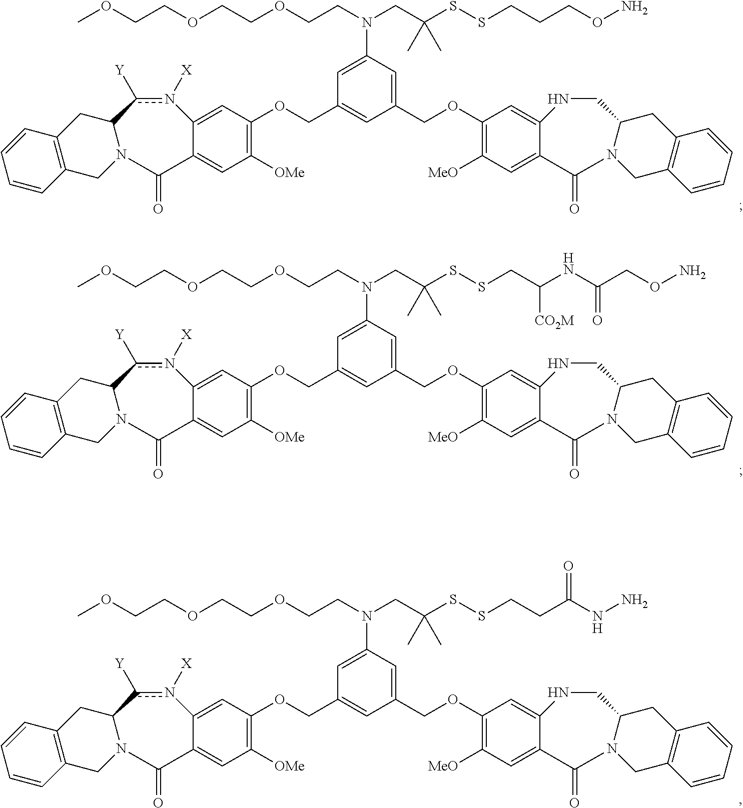

or a pharmaceutically acceptable salt thereof, wherein the double line [symbol] between N and C represents a single bond or a double bond, provided that when it is a double bondm X is absent and Y is —H; and when it is a single bond, X is —H; and Y is —OH or -S₃M. In a more specific embodiment, the double line [symbol] between N and C represents a double bond, X is absent and Y is —H. In another more specific embodiment, the double line [symbol] between N and C represents a single bond, X is —H and Y is —SO₃M.

In a third embodiment, the cytotoxic compound of the present invention has a thiol reactive group that can form a covalent bond with or more thiol groups (—SH) of one or more cysteine residues located on the cell-binding agent.

In a 1^{st} specific embodiment, the cytotoxic compound of the third embodiment is represented by the following formula:

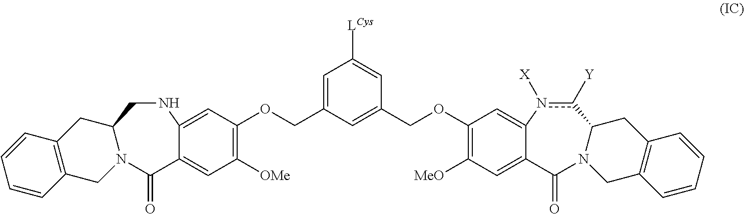

or a pharmaceutically acceptable salt thereof, wherein:

L^{Cys} is represented by the following formula:

—NR₅—P—C(═O)—(CRₐR_{b})ₘ—C(═OO)-L_{c}^{Cys} (C1);

—NR^{e′}—R^{x3}—C(═O)-L_{c}^{Cys} (C2);

—NR^{e}—C(═O)—R^{x1}—S-L_{c}^{Cys} (C3)

—NR^{e′}—R^{x2}—S-L_{c}^{Cys} (C4)

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃M, and M is H⁺ or a cation;

R₅ is —H or a (C₁-C₃)alkyl;

P is an amino acid residue or a peptide containing 2 to 20 amino acid residues;

Rₐ and R_{b}, for each occurrence, are independently —H, (C₁-C₃)alkyl, or a charged substituent or an ionizable group Q;

R^{e′} is —(CH₂—CH₂—O)ₙ—R^{k};

n is an integer from 2 to 6;

R^{k} is —H or -Me;

R^{x3} is a (C₁-C₆)alkyl;

L_{c}^{Cys} is represented by:

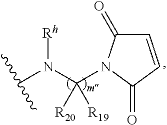

R₁₉ and R₂₀, for each occurrence, are independently —H or a (C₁-C₃)alkyl;

m″ is an integer between 1 and 10; and

R^{h} is —H or a (C₁-C₃)alkyl.

R^{x1} is a (C₁-C₆)alkyl;

R^{e} is —H or a (C₁-C₆)alkyl;

R^{x2} is a (C₁-C₆)alkyl;

L_{c}^{Cys} is represented by the following formula:

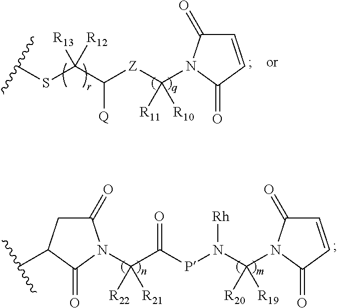

wherein:

Z is —C(═O)—NR₉—, or —NR₉—C(═O)—;

Q is —H, a charged substituent, or an ionizable group;

R₉, Ro₁₀, R₁₁, R₁₂, R₁₃, R₁₉, R₂₀, R₂₁ and R₂₂, for each occurrence, are independently —H or a (C₁-C₃)alkyl;

q and r, for each occurrence, are independently an integer between 0 and 10;

m and n are each independently an integer between 0 and 10;

R^{h} is —H or a (C₁-C₃)alkyl; and

P′ is an amino acid residue or a peptide containing 2 to 20 amino acid residues.

In a 2^{nd} specific embodiment, L^{Cys} is represented by formula (C1); and the remaining variables are as described above in the 1^{st} specific embodiment.

In a 3^{rd} specific embodiment, LC^{ys} is represented by formula (C2); and the remaining variables are as described above in the 1^{st} specific embodiment.

In a 4^{th} specific embodiment, for formula (C1); Rₐ and R_{b} are both H; and R₅ is H or Me; and the remaining variables are as described above in the 1^{st} or 2^{nd} specific embodiment.

In a 5^{th} specific embodiment, for formula (C1), P is a peptide containing 2 to 5 amino acid residues; and the remaining variables are as described above in the 1^{st}, 2^{nd} or 4^{th} specific embodiment. In a more specific embodiment, P is selected from Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), β-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. In another more specific embodiment, P is Gly-Gly-Gly, Ala-Val, Ala-Ala, Ala-D-Ala, D-Ala-Ala, or D-Ala-D-Ala.

In a 6^{th} specific embodiment, for formula (C1), Q is —SO₃M; and the remaining variables are as describe above in the 1^{st}, 2^{nd}, 4^{th} or 5^{th} specific embodiment or any more specific embodiments described therein.

In a 7^{th} specific embodiment, for formulae (C1) and (C2), R₁₉ and R₂₀ are both H; and m″ is an integer from 1 to 6; and the remaining variables are as described above in the 1^{st}, 2^{nd}, 3^{rd}, 4^{th}, 5^{th} or 6^{th} specific embodiment or any more specific embodiments described therein.

In a 8^{th} specific embodiment, for formulae (C1) and (C₂), -L_{C}^{Cys} is represented by the following formula:

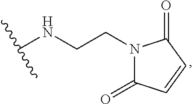 [SMILES: *NCCN1C(=O)C=CC1=O]

and the remaining variables are as described above in the 1^{st}, 2^{nd}, 3^{rd}, 4^{th}, 5^{th}, 6^{th} or 7^{th} specific embodiment or any more specific embodiments described therein.

In a 9^{th} specific embodiment, the cytotoxic compound of the third embodiment is represented by the following formula:

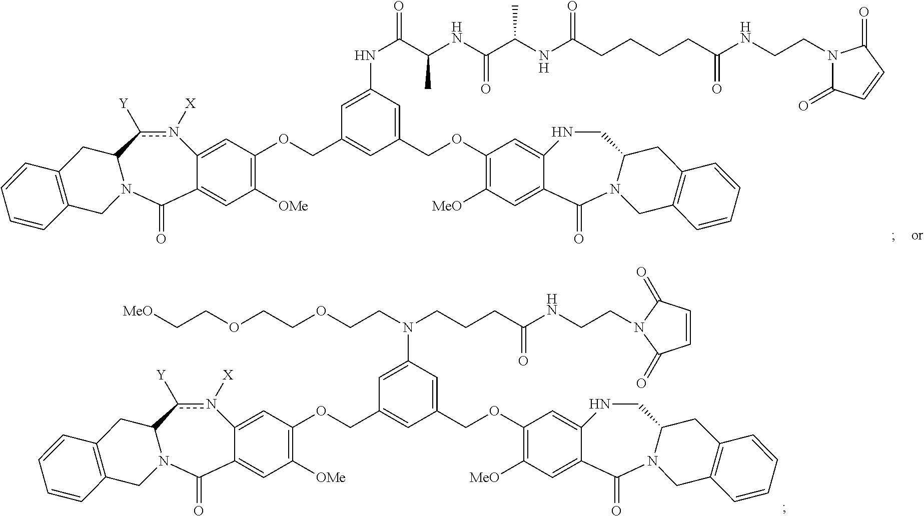

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃M. In a more specific embodiment, the double line [symbol] between N and C represents a double bond, X is absent and Y is —H. In another more specific embodiment, the double line [symbol] between N and C represents a single bond, X is —H and Y is —SO₃M.

In a 10^{th} specific embodiment, L^{Cys} is represented by formula (C3) or (C4), and the remaining variables are as described in the 1^{st} specific embodiment.

In a more specific embodiment, q and r are each independently an integer between 1 to 6, more specifically, an integer between 1 to 3. Even more specifically, R₁₀, R₁₁, R₁₂ and R₁₃ are all H.

In another more specific embodiment, m and n are each independently an integer between 1 and 6, more specifically, an integer between 1 to 3. Even more specifically, R₁₉, R₂₀, R₂₁ and R₂₂ are all H.

In a 11^{th} specific embodiment, L^{Cys} is represented by formula (C3); and the remaining variables are as described above in the 10^{th} specific embodiment or any more specific embodiments described therein.

In a 12^{th} specific embodiment, L^{Cys} is represented by formula (C4); and the remaining variables are as described above in the 10^{th} specific embodiment.

In a 13^{th} specific embodiment, for formula (C3) or (C4), P′ is a peptide containing 2 to 5 amino acid residues; and the remaining variables are as described in the 10^{th}, 11^{th} or 12^{th} specific embodiment or any more specific embodiments described therein. In a more specific embodiment, P′ is selected from Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), f3-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. In another more specific embodiment, P′ is Gly-Gly-Gly, Ala-Val, Ala-Ala, Ala-D-Ala, D-Ala-Ala, or D-Ala-D-Ala.

In a 14^{th} specific embodiment, for formula (C3) or (C4), -L^{Cys} is represented by the following formula:

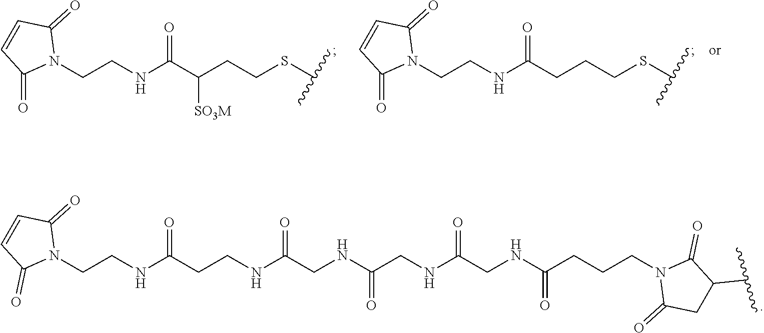

In a 15^{th} specific embodiment, for formula (C3) or (C4), R^{e} is H or Me; R^{x1} is —(CH₂)ₚ—(CR^{f}R^{g})—, and R^{x2} is —(CH₂)ₚ—(CR^{f}R^{g})—, wherein R^{f} and R^{g} are each independently —H or a (C₁-C₄)alkyl; and p is 0, 1, 2 or 3; and the remaining variables are as described above in the 10^{th}, 11^{th}, 12^{th}, 13^{th}, or 14^{th} specific embodiment. More specifically, R^{f} and R^{g} are the same or different, and are selected from —H and -Me.

In a 16^{th} specific embodiment, the cytotoxic compound of the third embodiment is represented by the following formula:

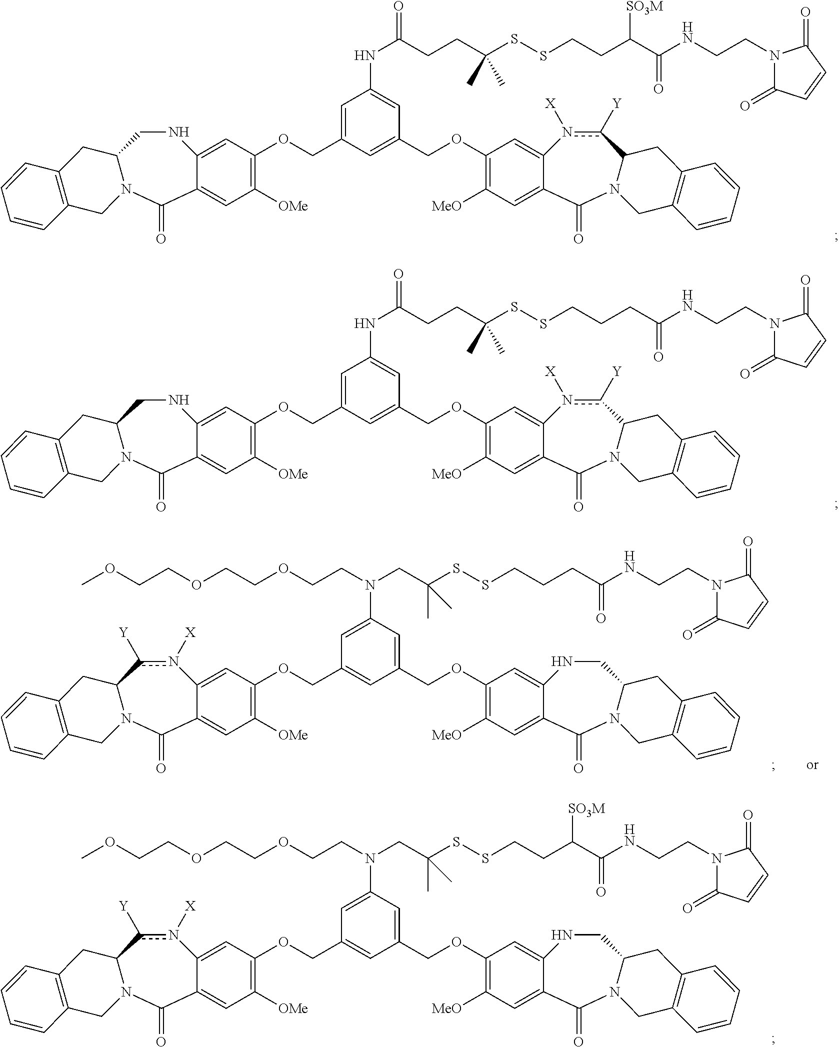

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃M. In a more specific embodiment, the double line [symbol] between N and C represents a double bond, X is absent and Y is —H. In another specific embodiment, the double line [symbol] between N and C represents a single bond, X is —H and Y is —SO₃M.

In some aspect, radio-labeled compounds of the present invention (e.g., compounds of formulae (I), (IA), (IB) or (IC)) could be useful in radio-imaging, in in vitro assays or in in vivo assays. “Isotopically” or “radio-labeled” compounds are identical to compounds disclosed herein in (e.g., compounds of formulae (I), (IA), (IB) or (IC)), but for the fact that one or more atoms are replaced or substituted by an atom having an atomic mass or mass number different from the atomic mass or mass number typically found in nature (i.e., naturally occurring). Suitable radionuclides that may be incorporated in compounds include, but are not limited to, ²H (also written as D for deuterium), ³H (also written as T for tritium), C, ¹³C, ¹⁴C ¹³N, ¹⁵N, ¹⁵O, ¹⁷O, ¹⁸O, ¹⁸F, ³⁵S, ³⁶Cl, ⁷⁵Br, 76Br, ⁷⁷Br, ⁸²Br, ¹²³I, ¹²⁴I, ¹²⁵I, or ¹³¹I. In some embodiments, the radionuclide is ³H, ¹⁴C, ³⁵S, ⁸²Br or ¹²⁵I. In some embodiments, the radionuclide is ³H or ¹²⁵I. Synthetic methods for incorporating radio-isotopes into organic compounds are applicable to compounds of the invention and are well known in the art. Examples of synthetic methods for the incorporation of tritium into target molecules are catalytic reduction with tritium gas, reduction with sodium borohydride or reduction with lithium aluminum hydride or tritium gas exposure labeling. Examples of synthetic methods for the incorporation of ¹²⁵I into target molecules are Sandmeyer and like reactions, or aryl or heteroaryl bromide exchange with ¹²⁵I.

In certain embodiment, for the compounds described herein (e.g., compounds of formula (I), (IA), (IB) or (Ic)), wherein the double line [symbol] between N and C represents a single bond, X is —H and Y is —SO₃M, the compounds is prepared by reacting the compound described herein, wherein the double line [symbol] between N and C represents a single bond, X is —H and Y is H, with a sulfonating agent. In a specific embodiment, the sulfonating agent is NaHSO₃ or KHSO₃. In another specific embodiment, the compound he compounds described herein (e.g., compounds of formula (I), (IA), (IB) or (Ic)), wherein the double line [symbol] between N and C represents a single bond, X is —H and Y is —SO₃M, is prepared by reacting the compound described herein, wherein the double line [symbol] between N and C represents a single bond, X is —H and Y is H, with a sulfonating agent in situ without purification before the resulting compound is reacted with the cell-binding agent. In one embodiment, the sulfonation reaction is carried out in an aqueous solution at a pH of 1.9 to 5.0, 2.9 to 4.0, 2.9 to 3.7, 3.1 to 3.5, 3.2 to 3.4. In a specific embodiment, the sulfonation reaction is carried out in an aqueous solution at pH 3.3. In one embodiment, the sulfonation reaction is carried out in dimethylacetamide (DMA) and water.

Cell-Binding Agent-Cytotoxic Agent Conjugates

In a second aspect, the present invention also provide cell-binding agent-cytotoxic agent conjugates comprising a cell-binding agent described herein covalently linked to one or more moleculars of the cytotoxic compounds described herein.

In some embodiments, the conjugate of the present invention is represented by the following formula:

CBACy)_{w} (III),

or a pharmaceutically acceptable salt thereof, wherein:

CBA is a cell-binding agent;

Cy is a cytotoxic agent represented by the following formula:

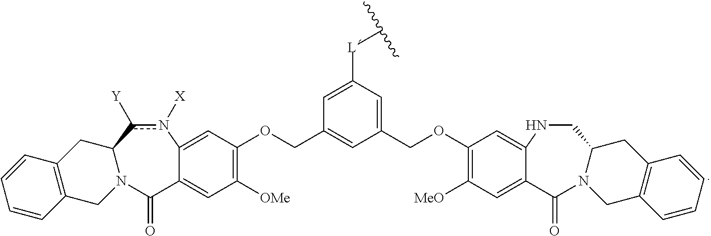

or a pharmaceutically acceptable salt thereof, wherein:

the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;

L′ is represented by the following formula:

—NR₅—P—C(═O)—W-J′ (L1′);

—NR₅—P—C(═O)—W—S—Z^{s1} (L2′);

—N(R^{e′})—W—S—Z^{s1} (L3′);

—N(R^{e})—C(═)—W—S—Z^{s1} (L4′); or

—N(R^{e′})—W-J′ (L5′);

R₅, for each occurrence, is independently H or a (C₁-C₃)alkyl;

W is a spacer unit;

J′ is a linking moiety;

R^{e} is H or a (C₁-C₃)alkyl;

R^{e′} is —(CH₂—CH₂—O)ₙ—R^{k};

n is an integer from 2 to 6;

R^{k} is H or Me;

Z^{s1} is a bifunctional linker covalently linked to the cytotoxic agent and the CBA; and

w is an integer from 1 to 20.

In a more specific embodiment, W is an optionally substituted linear, branched or cyclic alkyl, alkenyl, alkynyl, an aryl, a heteroaryl, or a heterocyclyl.

In another more specific embodiment, J′ is-C(═O)—.

In a first embodiment of the second aspect, the conjugates of the present invention comprises the cytotoxic compound covalently linked with the ε-amino group of one or more lysine residues located on the cell-binding agents described herein.

In a 1^{st} specific embodiment, the conjugate of the present invention is represented by the following formula:

CBACy^{Lys})_{w}_{_{L}} (IIIA)

wherein:

CBA is a cell-binding agent that is covalently linked through a lysine residue to Cy^{Lys};

Cy^{Lys} is represented by the following formula:

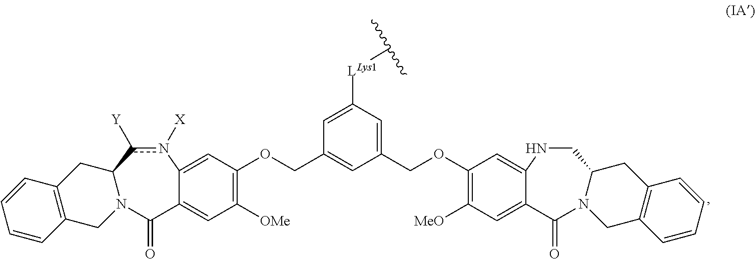

or a pharmaceutically acceptable salt thereof, wherein:

L^{Lys1} is represented by the following formula:

—NR₅—P—C(═O)—(CR^{a}R^{b})ₘ—C(═O)— (L1′);

—NR₅—P—C(═O)—(CR^{a}R^{b})ₘ—S—Z^{s1} (L2′);

—N(R^{e})—C(═O)—R^{x1}—S—Z^{s1} (L3′);

—N(R^{e′})—R^{x2}—S—Z^{s1} (L4′);

—N(R^{e′})—R^{x3}—C(═O)— (L5);

Z^{s1} is selected from any one of the following formulae:

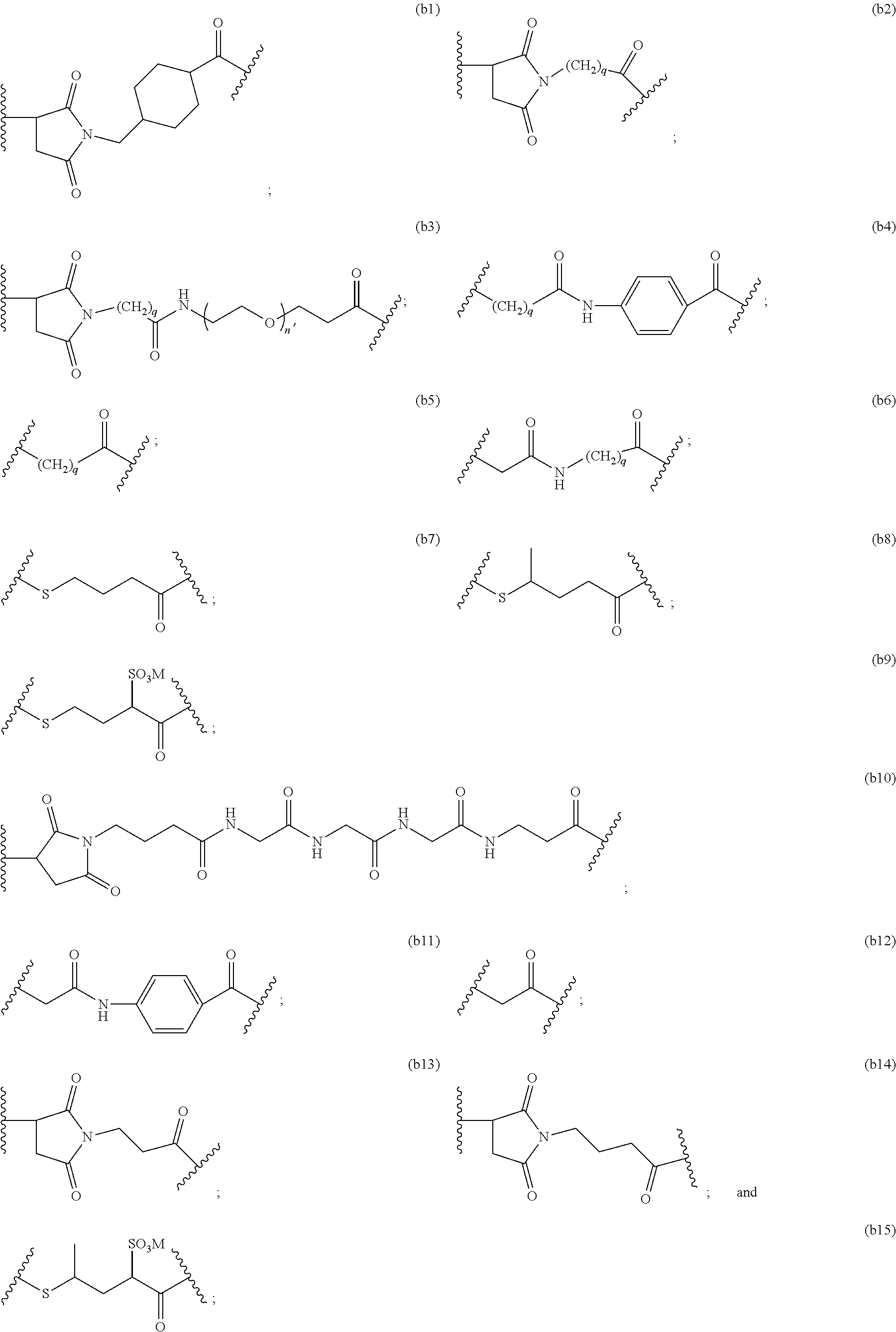

and the remaining variables are described above for formula (IA) in the 1^{st} specific embodiment of the first aspect.

In a 2^{nd} specific embodiment, L^{Lys1} is represented by formula (L1′) or (L2′); and the remaining variables are as described above in the 1^{st} specific embodiment.

In a 3^{rd} specific embodiment, L^{Lys1} is represented by formula (L5′); and the remaining variables are as described above in the 1^{st} specific embodiment. More specifically, R^{x3} is a (C₂-C₄)alkyl.

In a 4^{th} specific embodiment, for formulae (L1′) and (L2′), Rₐ and R_{b} are both H; R₅ is H or Me, and the remaining variables are as described above in the 1^{st} specific embodiment.

In a 5^{th} specific embodiment, for formulae (L′) and (L2′), P is a peptide containing 2 to 5 amino acid residues; and the remaining variables are described above in the 1^{st}, 2^{nd} or 4^{th} specific embodiment. In a more specific embodiment, P is selected from the group consisting of Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), β-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. More specifically, P is Gly-Gly-Gly, Ala-Val, Ala-Ala, Ala-D-Ala, D-Ala-Ala, or D-Ala-D-Ala.

In a 6^{th} specific embodiment, Q is —SO₃M; and the remaining variables are as described above in the 1^{st}, 2^{nd}, 4^{th} or 5^{th} specific embodiment or any more specific embodiments described therein.

In a 7^{th} specific embodiment, for formulae (L2′), (L3′) and (L4′), Z^{s1} is selected from any one of the following formulae:

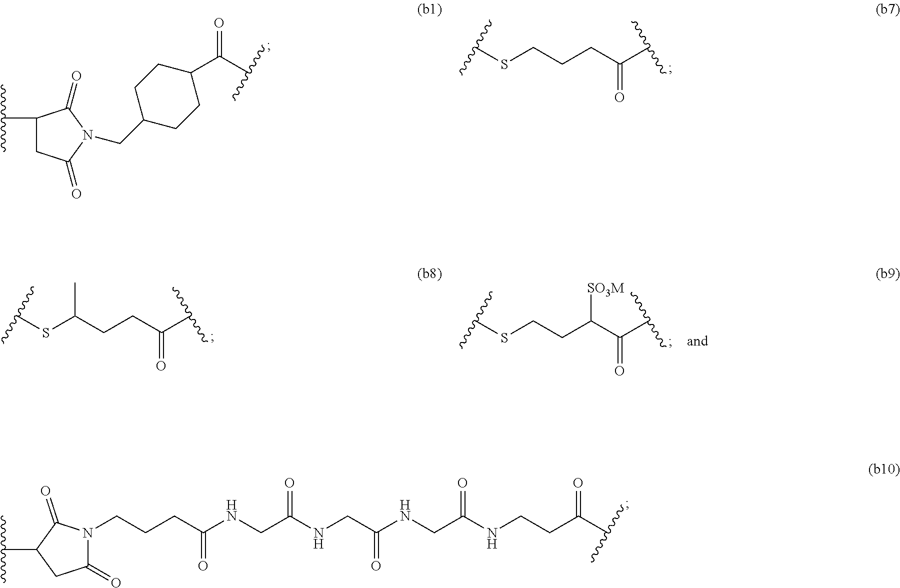

and the remaining variables are as described in the 1^{st}, 2^{nd}, 4^{th}, 5^{th} or 6^{th} specific embodiment or any more specific embodiments described therein.

In a 8^{th} specific embodiment, for formula (IA′), the double line [symbol] between N and C represents a double bond, X is absent and Y is —H; and the remaining variables are as described in the 1^{st}, 2^{nd}, 3^{rd}, 4^{th}, 5^{th}, 6^{th}, or 7^{th} specific embodiment or any more specific embodiments described therein.

In a 9^{th} specific embodiment, for formula (IA′), the double line [symbol] between N and C represents a single bond, X is H and Y is —SO₃M; and the remaining variables are as described in the 1^{st}, 2^{nd}, 3^{rd}, 4^{th}, 5^{th}, 6^{th}, or 7^{th} specific embodiment or any more specific embodiments described therein.

In a 10^{th} specific embodiment, for formula (IA′), the double line [symbol] between N and C represents a single bond or a double bond, provided that when it is a double bond, X is absent and Y is —H; and when it is a single bond, X is —H, and Y is —SO₃M;

M is H, N⁺ or K⁺;

L^{Lys1} is represented by the following formula:

—NR₅—P—C(═O)—(CR^{a}R^{b})ₘ—C(═O)— (L1′);

wherein:
R^{a} and R^{b} are both —H;
m is 3 to 5;
P is Ala-Ala, Ala-D-Ala, D-Ala-Ala, or D-Ala-D-Ala; and
R₅ is H or Me.

In a 11^{th} specific embodiment, for conjugates of formula (IIIA), the double line [symbol] between N and C represents a single bond or a double bond, provided that when it is a double bond, X is absent and Y is —H; and when it is a single bond, X is —H, and Y is-SO₃M;

M is H, Na⁺ or K⁺;

L^{Lys1} is represented by the following formula:

—NR₅—P—C(═O)—(CR^{a}R^{b})ₘ—S—Z^{s1} (L2′),

wherein:
(CR^{a}R^{b})ₘ— is —(CH₂)ₚ—(CR^{f}R^{g})—, wherein R^{f} and R^{g} are each independently —H or -Me; and p is 0, 1, 2 or 3;
P is Ala-Ala, Ala-D-Ala, D-Ala-Ala, or D-Ala-D-Ala;
R is H or Me; and
Z^{s1} is H, —SR^{d} or is represented by formula (b1), (b7), (b8), (b9) or (b10).

In a 12^{th} specific embodiment, for formula (IA′), the double line [symbol] between N and C represents a single bond or a double bond, provided that when it is a double bond, X is absent and Y is —H; and when it is a single bond, X is —H, and Y is —SO₃M;

M is H, N⁺ or K⁺;

L^{Lys1} is represented by the following formula:

—N(R^{e})—C(═O)—R^{x1}—S—Z^{s1} (L3′);

wherein:
R^{e} is H or Me;
R^{x1} is —(CH₂)ₚ—(CR^{f}R^{g})—, wherein R^{f} and R^{g} are each independently —H or -Me; and p is 0, 1, 2 or 3;
Z^{s1} is represented by formula (b1), (b7), (b8), (b9) or (b10).

In a 13^{th} specific embodiment, for formula (IA′), the double line [symbol] between N and C represents a single bond or a double bond, provided that when it is a double bond, X is absent and Y is —H; and when it is a single bond, X is —H, and Y is —SO₃M;

M is H, N⁺ or K⁺;

L^{Lys1} is represented by the following formula:

—N(R^{e′})—R^{x2}—S—Z^{s1} (L4′);

wherein:
R^{x2} is —(CH₂)ₚ—(CR^{f}R^{g})—, wherein R^{f} and R^{g} are each independently —H or -Me; and p is 0, 1, 2 or 3;
R^{e′} is —(CH₂—CH₂—O)ₙ—R^{k};
R^{k} is Me;
Z^{s1} is represented by formula (b1), (b7), (b8), (b9) or (b10).

In a 14^{th} specific embodiment, for conjugates of formula (IIIA), the double line [symbol] between N and C represents a single bond or a double bond, provided that when it is a double bond, X is absent and Y is —H; and when it is a single bond, X is —H, and Y is —SO₃M;

M is H, Na⁺ or K⁺;

L^{Lys1} is represented by the following formula:

—N(R^{e′})—R^{x3}—C(═O)— (L5′);

wherein:
R^{e′} is —(CH₂—CH₂—O)ₙ—R^{k};
R^{k} is Me;
R^{x3} is —(CR^{a}R^{b})ₘ—
R^{a} and R^{b} are both —H;
m is 3 to 5.

In a 15^{th} specific embodiment, the conjugates of the first embodiment is represented by the following formula:

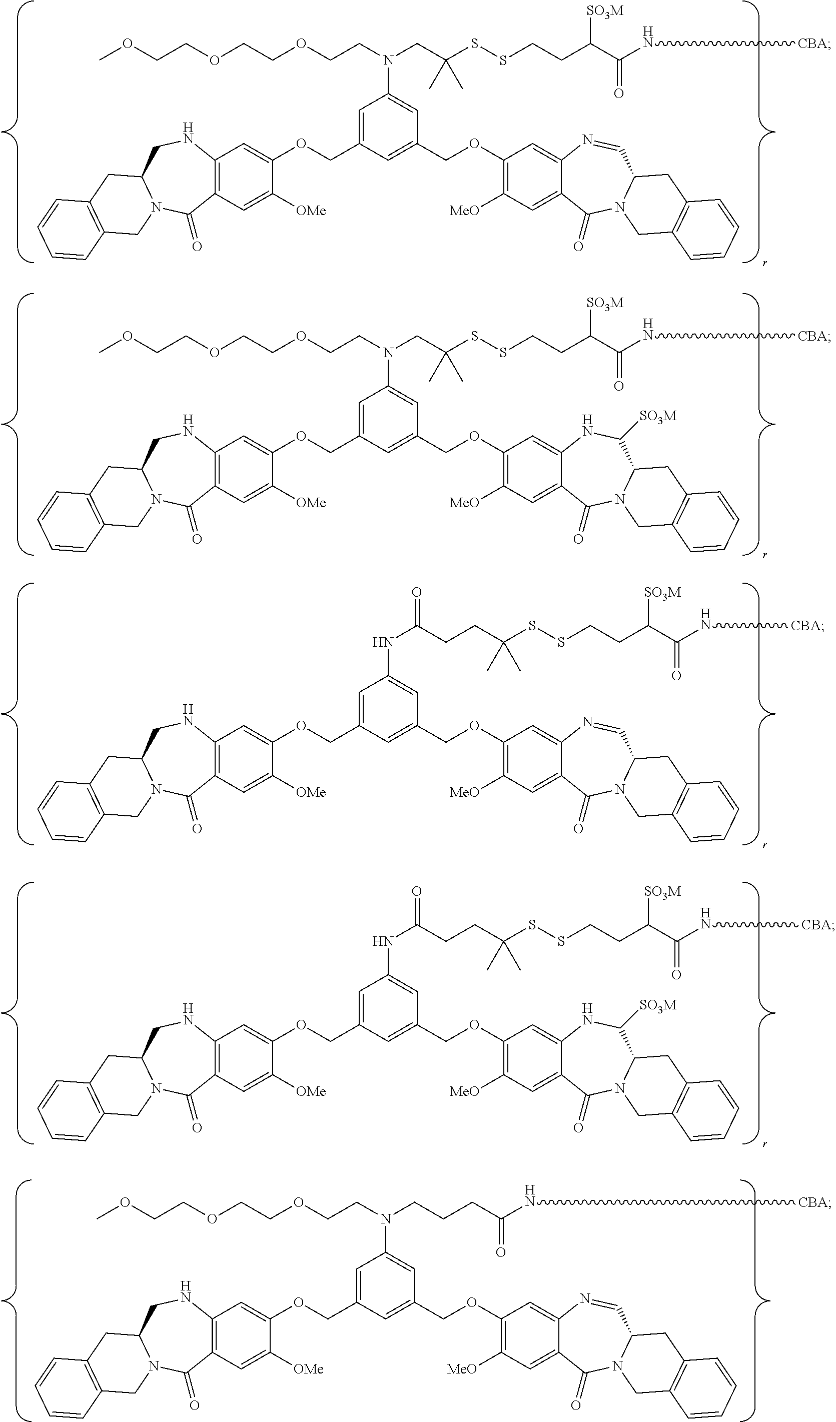

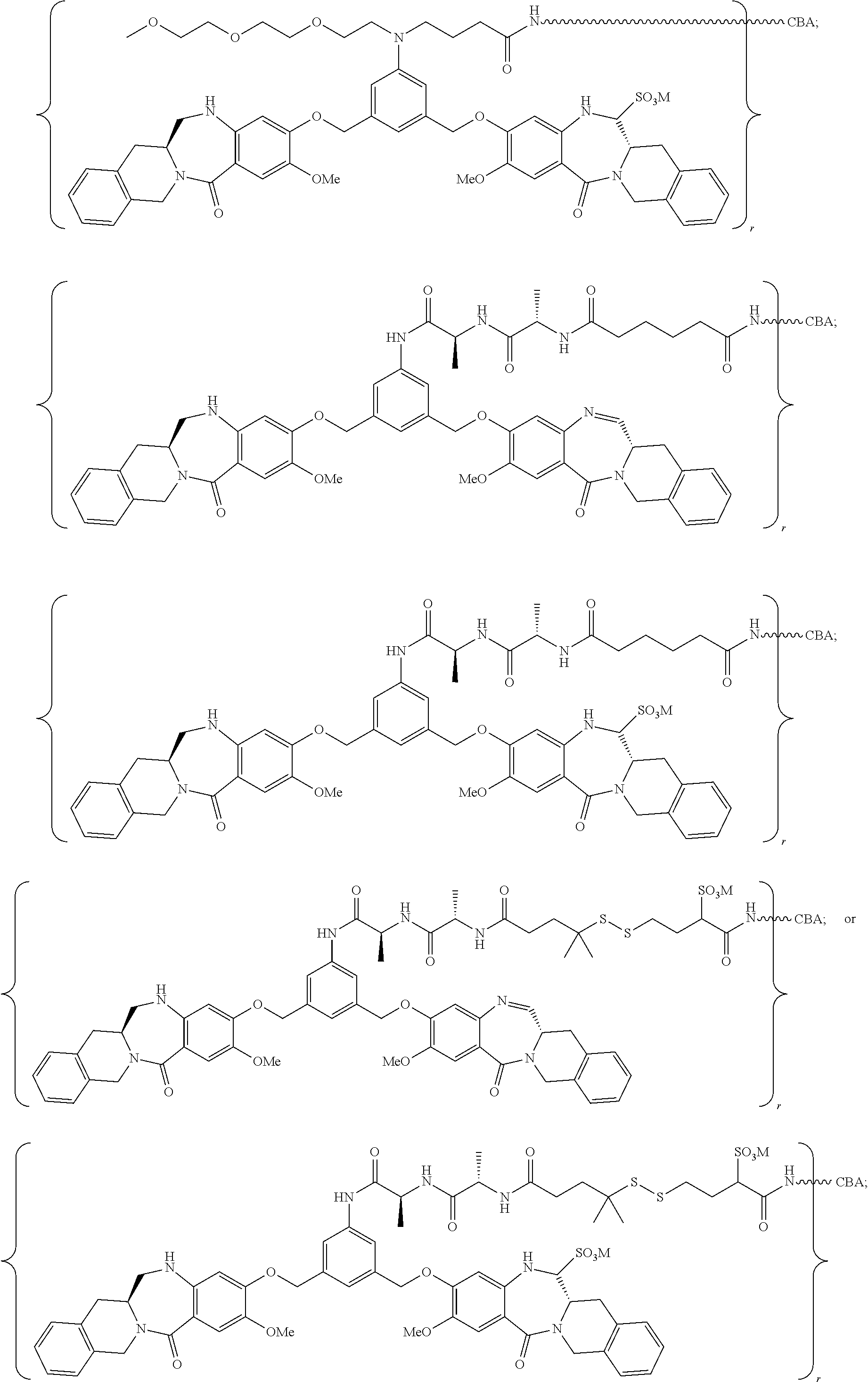

or a pharmaceutically acceptable salt thereof, wherein CBA [symbol] NH— represents the cell-binding agent that is covalently linked to the cytotoxic compound; M is H, Na⁺ or K⁺; and r is an integer from 1 to 10.

The conjugates described in the first embodiment or any specific embodiments descried therein can be prepared according to any methods known in the art, see, for example, WO 2012/128868 and WO2012/112687, which are incorporate herein by reference.

In some embodiments, the immunoconjugates of the first embodiment can be prepared by a first method comprising the steps of reacting the CBA with a cytotoxic agent having an amine reactive group.

In some embodiments, for the first method described above, the reaction is carried out in the presence of an imine reactive reagent, such as NaHSO₃.

In some embodiments, the conjugates of the first embodiment can be prepared by a second method comprising the steps of:

(a) reacting a cytotoxic agent with a linker compound having an amine reactive group and a thiol reactive group to form a cytotoxic agent-linker compound having the amine reactive group bound thereto; and

(b) reacting the CBA with the cytotoxic agent-linker compound.

In some embodiments, for the second method described above, the reaction in step (a) is carried out in the presence of an imine reactive reagent, such as NaHSO₃.

In some embodiments, for the second method described above, the cytotoxic agent-linker compound is reacted with the CBA without purification. Alternatively, the cytotoxic agent-linker compound is first purified before reacting with the CBA.

In another embodiment, the conjugates of the first embodiment can be prepared by a third method comprising the steps of:

(a) reacting the CBA with a linker compound having an amine reactive group and a thiol reactive group to form a modified CBA having a thiol reactive group bound thereto; and

(b) reacting the modified CBA with the cytotoxic agent.

In some embodiments, for the third method described above, the reaction in step (b) is carried out in the presence of an imine reactive reagent.

In another embodiment, the conjugates of the first embodiment can be prepared by a fourth method comprising the steps of reacting the CBA, a cytotoxic compound and a linker compound having an amine reactive group and a thiol reactive group.

In some embodiments, for the fourth method, the reaction is carried out in the presence of an imine reactive agent.

In a second embodiment, the conjugates of the present invention comprises a cell-binding agent (CBA) covalently linked to a cytotoxic compound described in the second embodiment of the first aspect through one or more aldehyde groups located on the CBA.

In a 1^{st} specific embodiment, the conjugate is represented by the following formula:

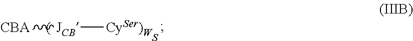

wherein:

CBA is the oxidized cell-binding agent described herein;

W_{S} is 1, 2, 3, or 4;

J_{CB}′ is a moiety formed by reacting an aldehyde group on the CBA with an aldehyde reactive group on Cy^{Ser}, and is represented by the following formula:

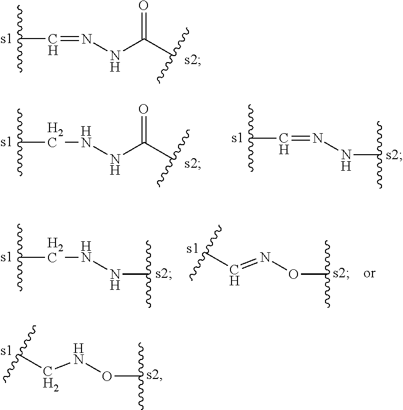

wherein s1 is the site covalently linked to the CBA; and s2 is the site covalently linked to Cy^{Ser}; and

Cy^{Ser} is represented by the following formula:

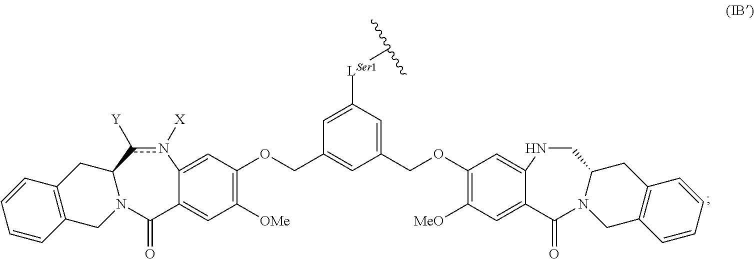

or a pharmaceutically acceptable salt thereof, wherein L^{Ser}:

—NR₅—P—C(═O)—(CR^{a}R^{b})ᵣ—Z_{d1}—(CR^{a}R^{b})_{r′}— (S1′); or

—N(R^{e′})—R^{x3}—C(═O)-L- (S2′);

—N(R^{e})—C(═O)—R^{x1}—S-L₁- (S3′)

—N(R^{e′})—R^{x2}—S-L₁- (S4′);

and the remaining variables are described above for formula (IB) in the first aspect.

In a 2^{nd} specific embodiment, L^{Ser1} is represented by formula (S1′); and the remaining variables are as described above in the 1^{st} specific embodiment.

In a 3^{rd} specific embodiment, L^{Ser1} is represented by formula (S2′); and the remaining variables are as described above in the 1^{st} specific embodiment. More specifically, R^{x3} is a (C₂-C₄)alkyl.

In a 4^{th} specific embodiment, for formula (S1′), Rₐ and R_{b} are both H, and R₅ and R₉ are both H or Me; and the remaining variables are as described above in the 1^{st} or 2^{nd} specific embodiment.

In a 5^{th} specific embodiment, for formula (S1′), P is a peptide containing 2 to 5 amino acid residues; and the remaining variables are as described above in the 1^{st}, 2^{nd} or 4^{th} specific embodiment. In a more specific embodiment, P is selected from the group consisting of Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), β-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. Even more specifically, P is Gly-Gly-Gly, Ala-Val, Ala-Ala, Ala-D-Ala, D-Ala-Ala, or D-Ala-D-Ala.

In a 6^{th} specific embodiment, for formula (S1′), Q is —SO₃M; and the remaining variables are as described above in the 1^{st}, 2^{nd}, 4^{th} or 5^{th} specific embodiment.

In a 7^{th} specific embodiment, the conjugates of the second embodiment is represented by the following formula:

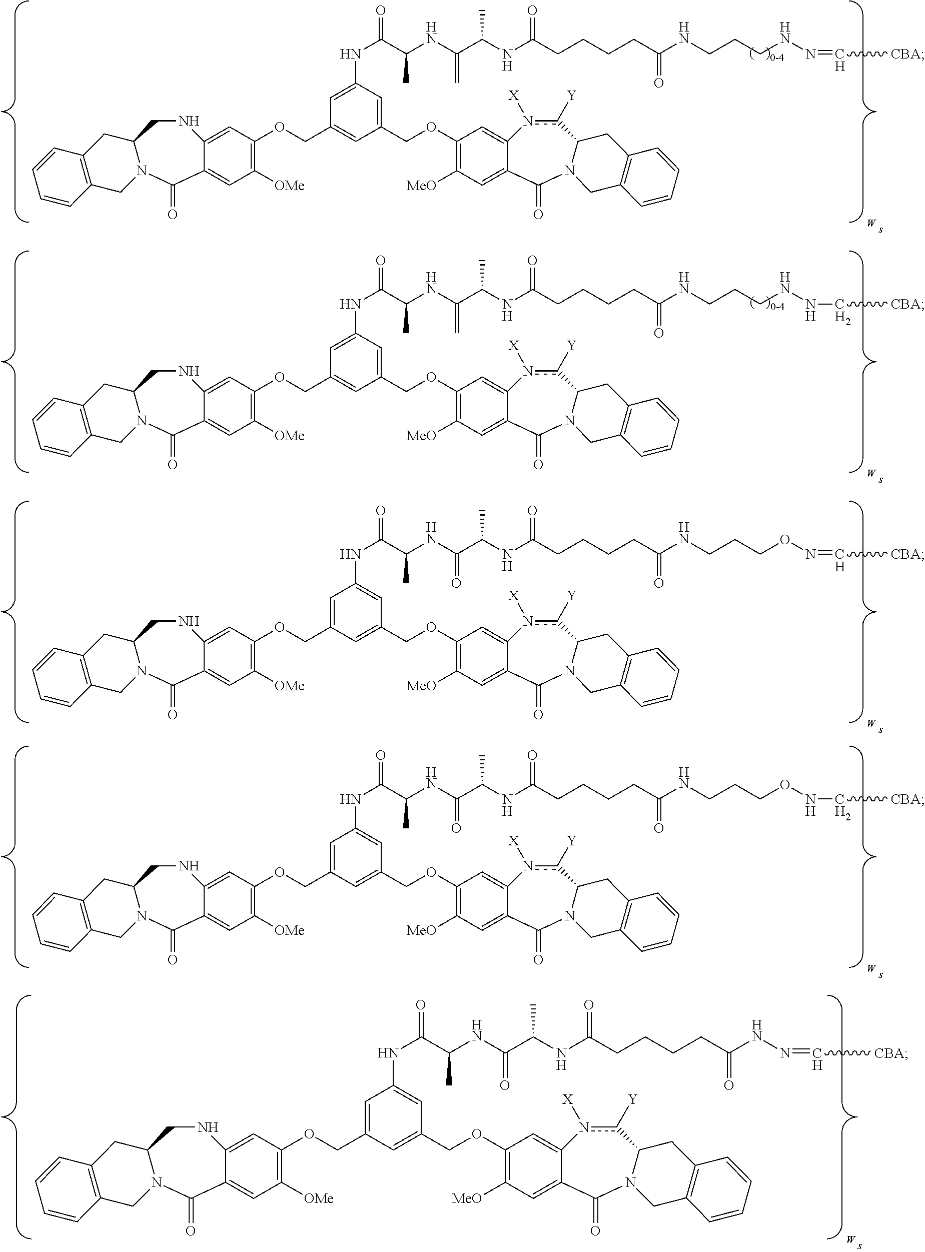

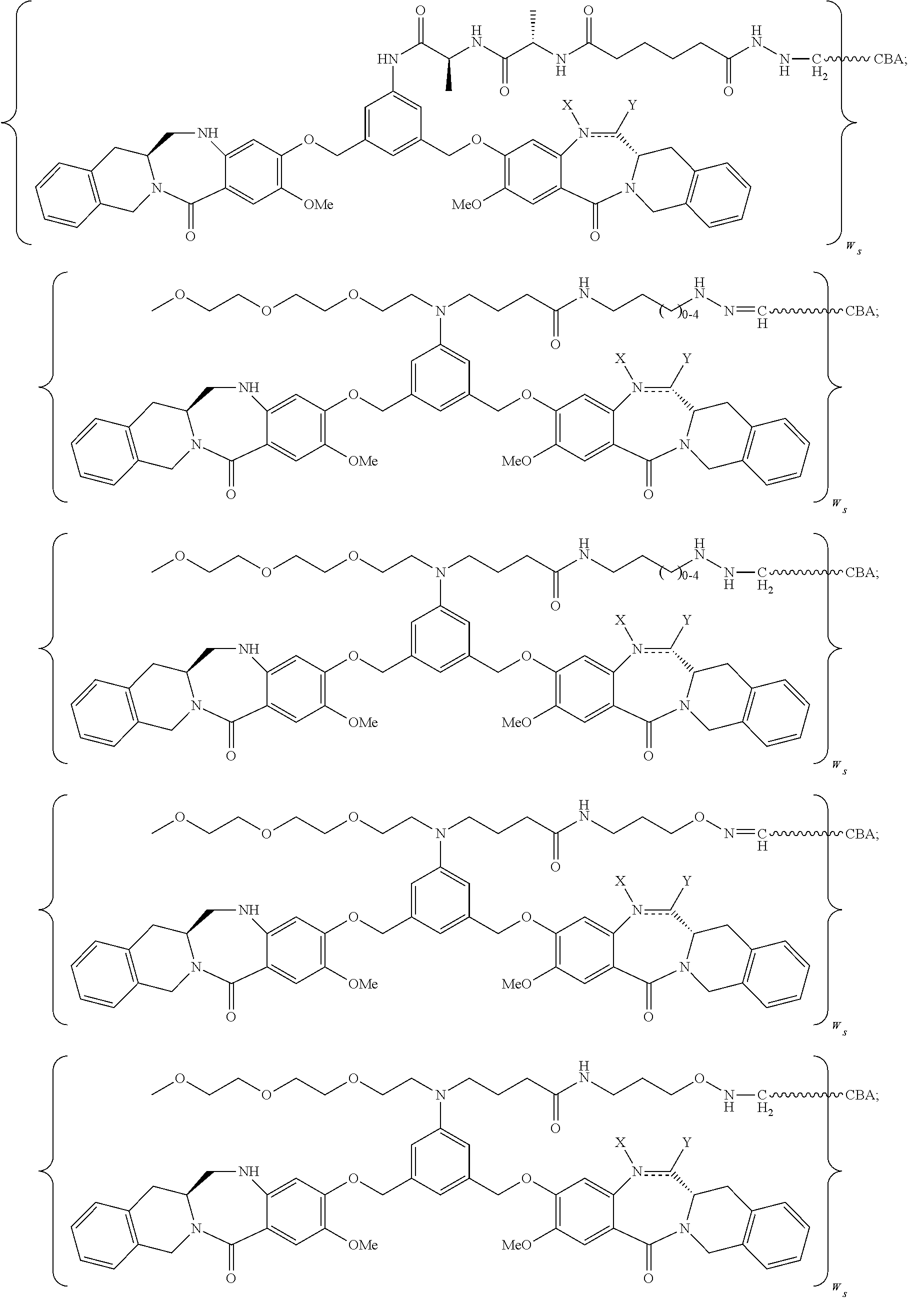

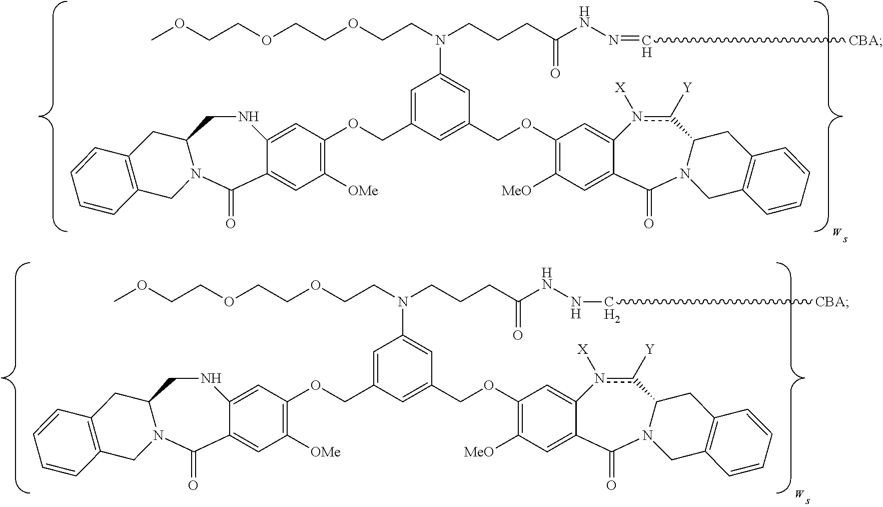

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or -SO₃M. In a more specific embodiment, the double line [symbol] between N and C represents a double bond, X is absent and Y is —H. In another more specific embodiment, the double line [symbol] between N and C represents a single bond, X is —H and Y is —SO₃M.

In an 8^{th} specific embodiment, L^{Ser1} is represented by formula (S3′) or (S4′), and the remaining variables as described above in the 1^{st} specific embodiment.

In a more specific embodiment, Zₐ₂ is absent; q1 and r1 are each independent an integer from 0 to 3, provided that q1 and r1 are not both 0; and the remaining variables are as described above in the 8^{th} specific embodiments. Even more specifically, Rₐ₁, Rₐ₂, Rₐ₃, Rₐ₄ are all —H.

In another more specific embodiment, Zₐ₂ is —C(═O)—NH—, or —NH₉—C(═O)—; q1 and r1 are each independently an integer from 1 to 6; and the remaining variables are as described above in the 8^{th} specific embodiments. Even more specifically, Rₐ₁, Rₐ₂, Rₐ₃, Rₐ₄ are all —H.

In a 9^{th} specific embodiment, L^{Ser1} is represented by formula (S3′); and the remaining variables are as described above in the 8^{th} specific embodiment or any more specific embodiments described therein.

In a 10^{th} specific embodiment, L^{Ser1} is represented by formula (S4′); and the remaining variables are as described above in the 8^{th} specific embodiment or any more specific embodiments described therein.

In an 11^{th} specific embodiment, for formula (S3′) and (S4′), -L₁- is represented by the following formula:

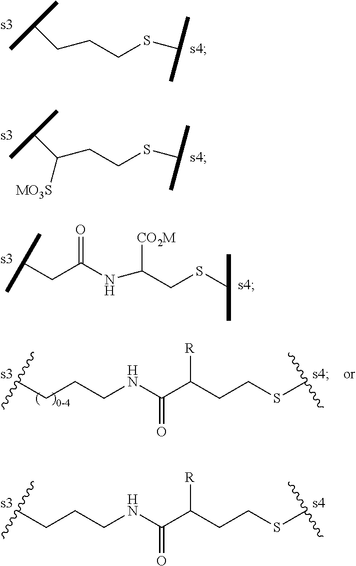

or a pharmaceutically acceptable salt thereof, wherein R is H or —SO₃M; and the remaining variables are as described above in the 8^{th}, 9^{th} or 10^{th} specific embodiment or any more specific embodiments described therein.

In a 12^{th} specific embodiment, for formula (S3′) or (S4′), R^{e} is H or Me; and R^{x1} is —(CH₂)ₚ—(CR^{f}R^{g})—, and R^{x2} is —(CH₂)ₚ—(CR^{f}R^{g})—, wherein R^{f} and R^{g} are each independently —H or a (C₁-C₄)alkyl; and p is 0, 1, 2 or 3. More specifically, R^{f} and R^{g} are the same or different, and are selected from —H and -Me.

In a 13^{th} specific embodiment, the conjugate of formula (IIIB) of the second embodiment is represented by the following formula:

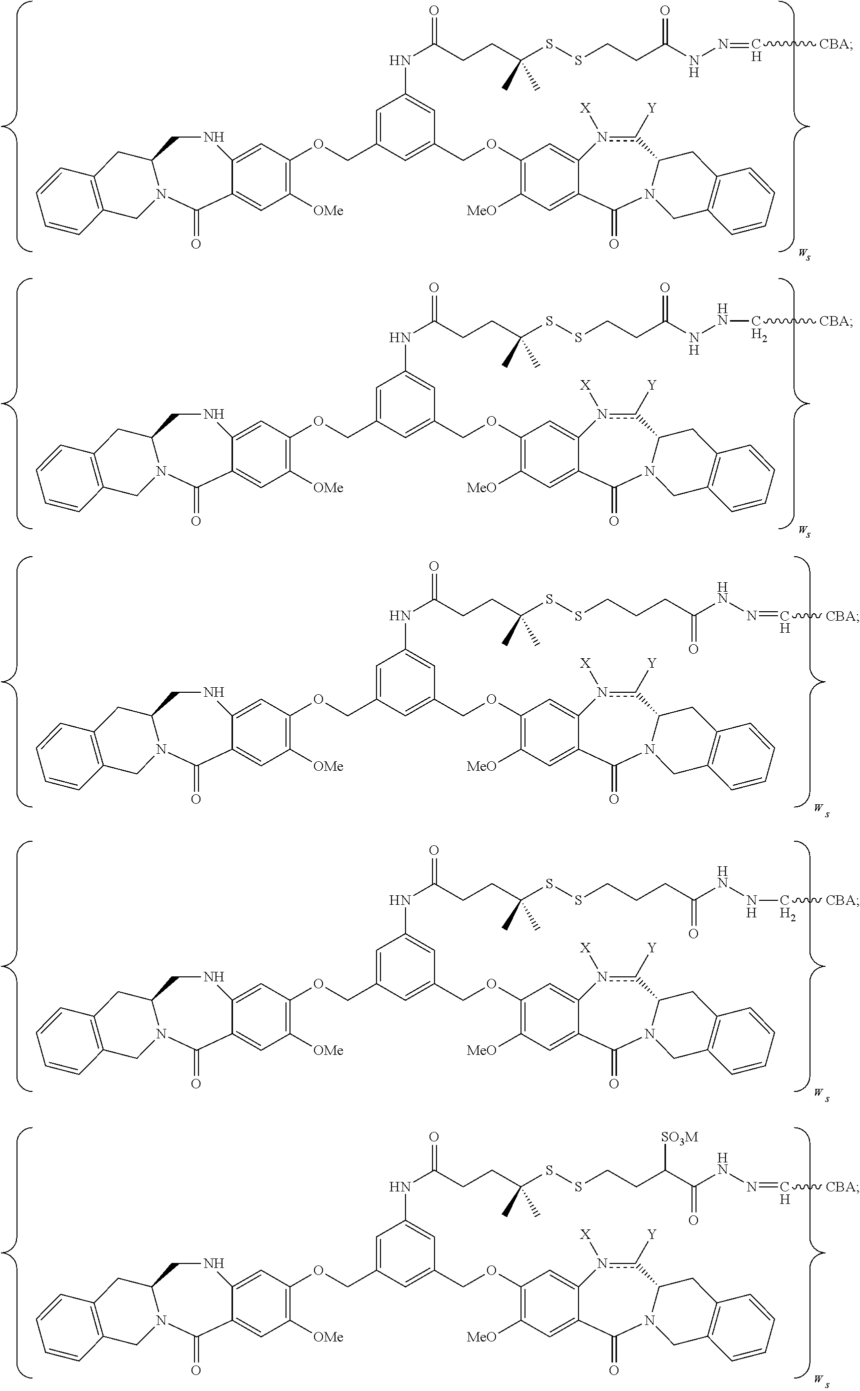

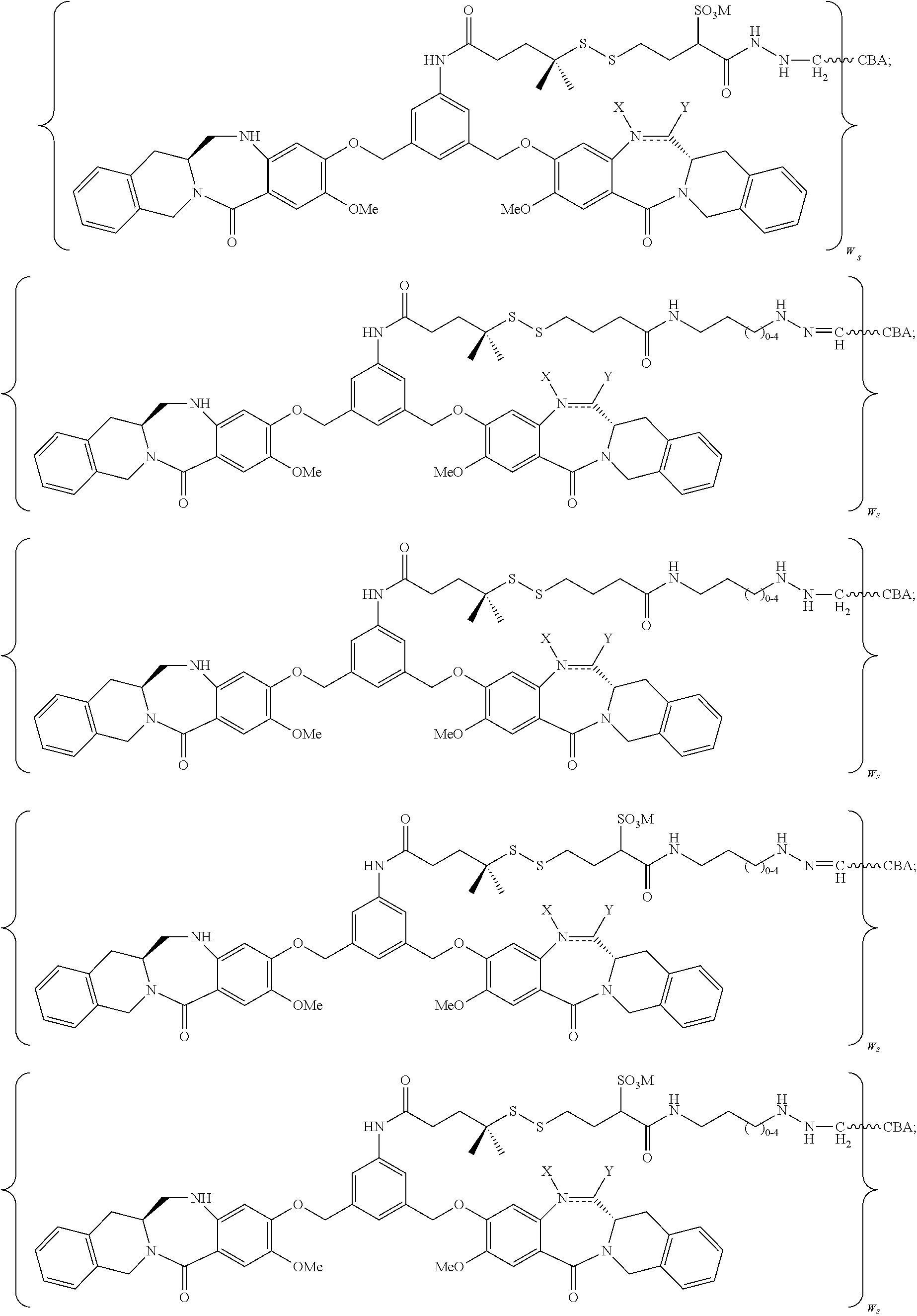

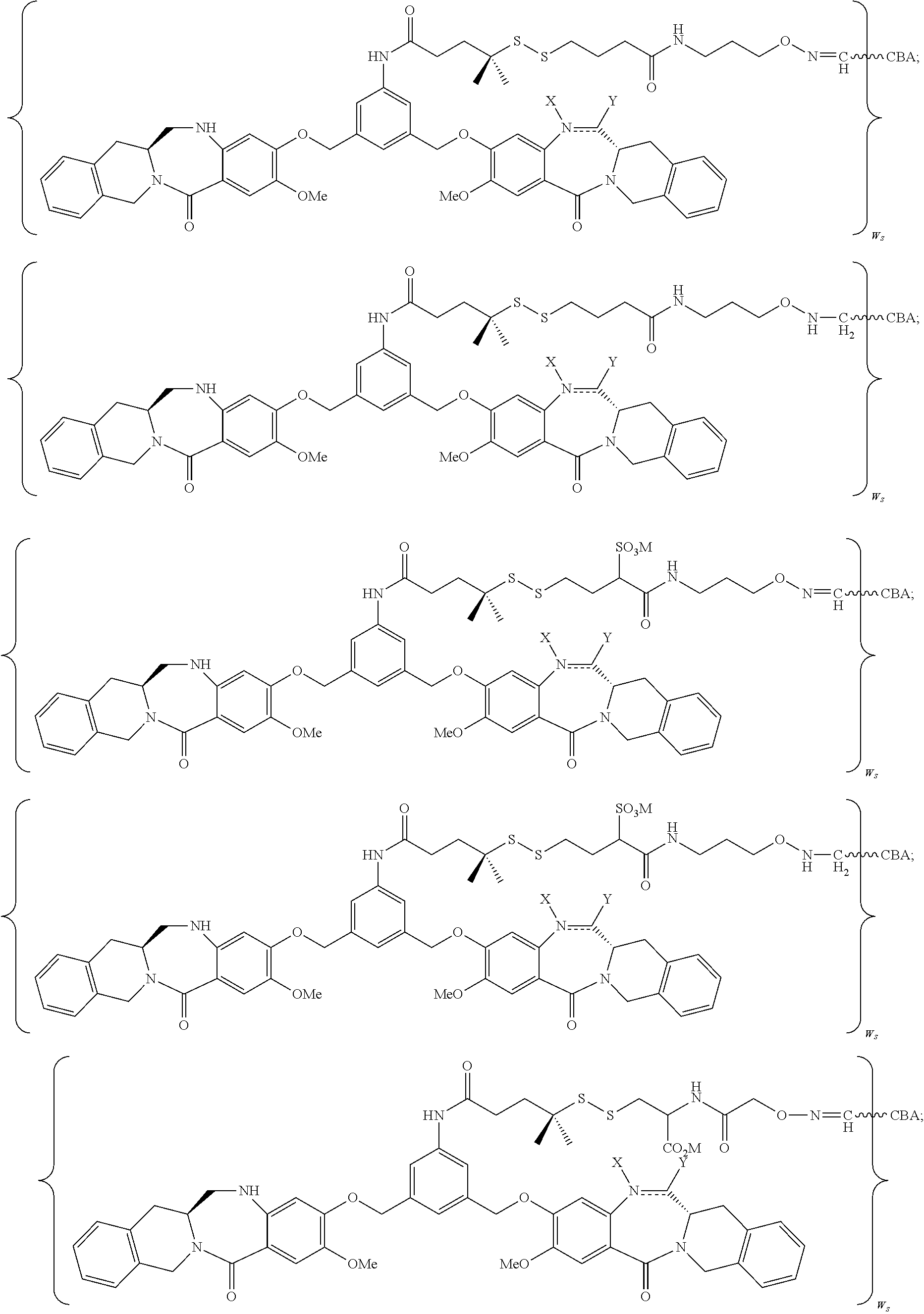

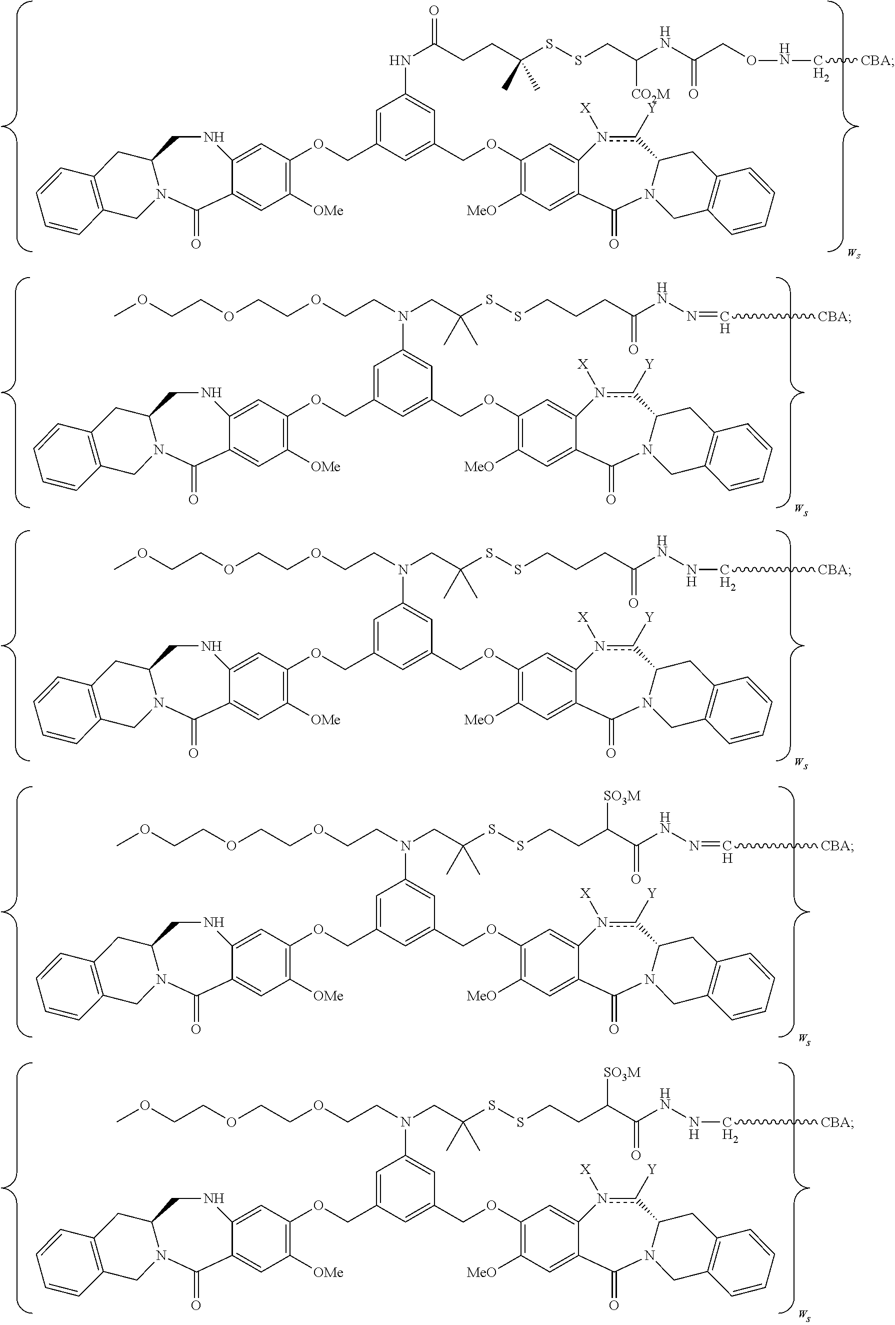

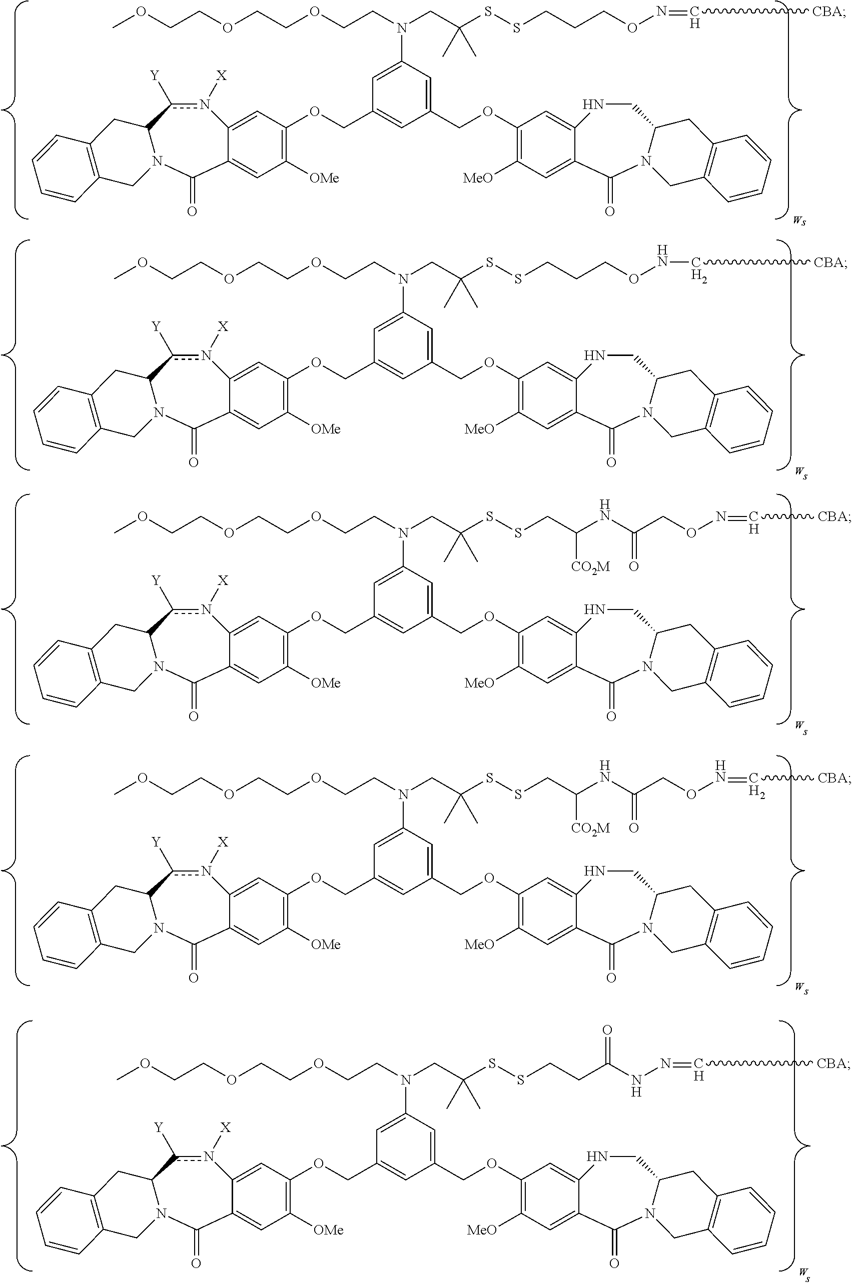

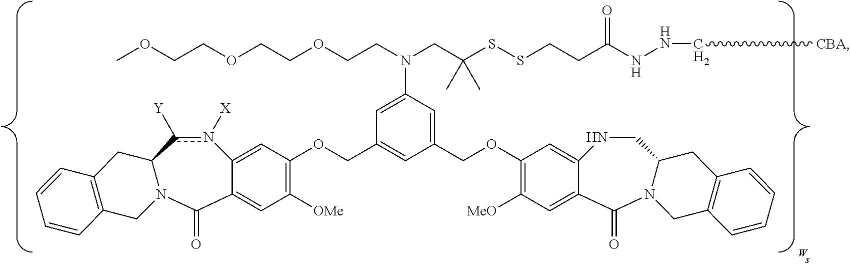

or a pharmaceutically acceptable salt thereof, wherein the double line [symbol] between N and C represents a single bond or a double bond, provided that when it is a double bond, X is absent and Y is —H; and when it is a single bond, X is —H; and Y is —OH or —SO₃M. In a more specific embodiment, the double line [symbol] between N and C represents a double bond, X is absent and Y is —H. In another more specific embodiment, the double line [symbol] between N and C represents a single bond, X is —H and Y is —SO₃M.

In any of the above 1^{st} to the 13^{th} specific embodiments, the subject oxidized cell-binding agent may have 1, 2, 3, or up to 4 N-terminal 2-hydroxyethylamine moieties oxidized to aldehyde group(s), for linking covalently to a cytotoxic agent described herein. The N-terminal 2-hydroxyethylamine moiety may be part of a serine, threonine, hydroxylysine, 4-hydroxyornithine or 2,4-diamino-5-hydroxy valeric acid residue, preferably Ser or Thr. For simplicity, the description below, including the oxidation reaction and any subsequent conjugation with linkers or cytotoxic agents, may refer to Ser as a specific example of such N-terminal 2-hydroxyethylamine moieties, but should generally be construed as referring to all N-terminal 2-hydroxyethylamine moieties.

In some embodiments, the conjugates of the second embodiment can be prepared by a first method comprising reacting an oxidized CBA having an N-terminal aldehyde described herein with a cytotoxic agent having an aldehyde reactive group.

In some embodiments, the conjugates of the second embodiment can be prepared by a second method comprising reacting an oxidized CBA agent having an N-terminal aldehyde described in the first aspect of the invention with a linker compound having an aldehyde reactive group to form a modified cell-binding agent having a linker bound thereto, followed by reacting the modified CBA with a cytotoxic agent.

In another embodiment, the conjugates of the second embodiment can be prepared by a third method comprising contacting an oxidized CBA having an N-terminal aldehyde described herein with a cytotoxic agent followed by addition of a linker compound having an aldehyde reactive group.

In another embodiment, the conjugates of the second embodiment can be prepared by a fourth method comprising the steps of:

(a) oxidizing a CBA having a N-terminal 2-hydroxyethylamine moiety (e.g., Ser/Thr) with an oxidizing agent to form an oxidized CBA having a N-terminal aldehyde group; and

(b) reacting the oxidized CBA having the N-terminal aldehyde group with a cytotoxic agent having an aldehyde reactive group.

In some embodiments, the conjugates of the second embodiment can be prepared by a fifth method comprising the steps of:

(a) oxidizing a CBA having a N-terminal 2-hydroxyethylamine moiety (e.g., Ser/Thr) with an oxidizing agent to form an oxidized CBA having a N-terminal aldehyde group;

(b) reacting the oxidized CBA having the N-terminal aldehyde group with a linker compound having an aldehyde reactive group to form a modified binding agent having a linker bound thereto, followed by reacting the modified CBA with a cytotoxic agent.

In another embodiments, the conjugates of the second embodiment can be prepared by a sixth method comprising the steps of:

(a) oxidizing the CBA having a N-terminal 2-hydroxyethylamine moiety (e.g., Ser/Thr) with an oxidizing agent to form an oxidized CBA having a N-terminal aldehyde group;

(b) contacting the oxidized CBA having the N-terminal aldehyde group with a cytotoxic agent followed by addition of a linker compound having an aldehyde reactive group.

Any suitable oxidizing agent can be used in step (a) of the methods described above. In certain embodiments, the oxidizing agent is a periodate. More specifically, the oxidizing agent is sodium periodate.

In a third embodiment, the conjugate of the present invention comprises a cell-binding agent (CBA) described herein covalently linked to a cytotoxic agent described herein through the thiol groups (—SH) of one or more cysteine residues located on the cell-binding agent.

In a 1^{st} specific embodiment, the conjugate of the third embodiment is represented by the following formula:

CBACy^{Cys})_{w}_{_{C}} (IIIC),

wherein:

w_{C} is 1 or 2;

Cy^{Cys} is represented by the following formula:

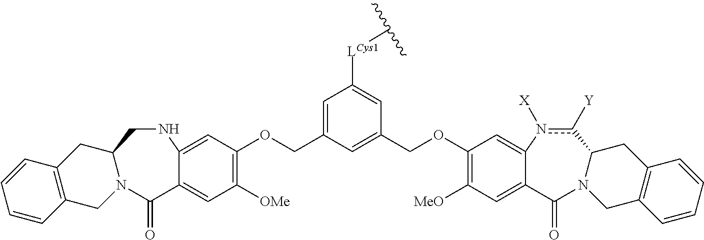

or a pharmaceutically acceptable salt thereof, wherein:

L^{Cys1} is represented by the following formula:

—NR₅—P—C(═O)—(CRₐR_{b})ₘ—C(═O)-L_{c}^{Cys1} (C1′);

—NR^{e′}—R^{x3}—C(═O)-L_{c}^{Cys1} (C2′);

—NR^{e}—C(═O)—R^{x1}—S-L_{c1}^{Cys1} (C3′)

—NR^{e′}—R^{x2}—S-L_{c1}^{Cys1} (C4′)

the double line [symbol] between N and C represents a single bond or a double bond, provided that when it is a double bond, X is absent and Y is —H or a (C₁-C₄)alkyl; and when it is a single bond, X is —H or an amine protecting moiety, Y is —OH or —SO₃M, and M is H⁺ or a cation;

R₅ is —H or a (C₁-C₃)alkyl;

P is an amino acid residue or a peptide containing 2 to 20 amino acid residues;

Rₐ and R_{b}, for each occurrence, are independently —H, (C₁-C₃)alkyl, or a charged substituent or an ionizable group Q;

W′ is —NR^{e′},

R^{e′} is —(CH₂—CH₂—O)ₙ—R^{k};

n is an integer from 2 to 6;

R^{k} is —H or -Me;

R^{x3} is a (C₁-C₆)alkyl; and,

L_{C}^{Cys1} is represented by:

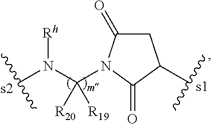

wherein s1 is the site covalently linked to CBA, and s2 is the site covalently linked to the —C(═O)— group on Cy^{Cys}

R₁₉ and R₂₀, for each occurrence, are independently —H or a (C₁-C₃)alkyl;

m″ is an integer between 1 and 10; and

R^{h} is —H or a (C₁-C₃)alkyl.

R^{x1} is a (C₁-C₆)alkyl;

R^{e} is —H or a (C₁-C₆)alkyl;

R^{k} is —H or -Me;

R^{x2} is a (C₁-C₆)alkyl;

L_{c1}^{Cys1} is represented by the following formula:

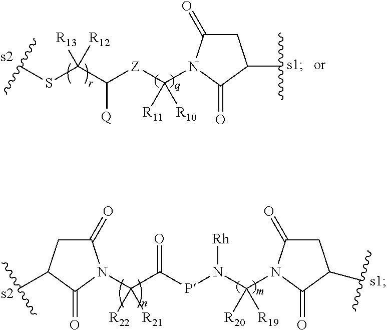

wherein:

s1 is the site covalently linked to the CBA and s2 is the site covalently linked to —S— group on Cy^{Cys};

Z is —C(═O)—NR₉—, or —NR₉—C(═O)—;

Q is —H, a charged substituent, or an ionizable group;

R₉, R₁₀, R₁₁, R₁₂, R₁₃, R₁₉, R₂₀, R₂₁ and R₂₂, for each occurrence, are independently —H or a (C₁-C₃)alkyl;

q and r, for each occurrence, are independently an integer between 0 and 10;

m and n are each independently an integer between 0 and 10;

R^{h} is —H or a (C₁-C₃)alkyl; and

P′ is an amino acid residue or a peptide containing 2 to 20 amino acid residues.

In a 2^{nd} specific embodiment, L^{Cys1} is represented by formula (C1′); and the remaining variables are as described above in the 1^{st} specific embodiment.

In a 3^{rd} specific embodiment, L^{Cys1} is represented by formula (C2′); and the remaining variables are as described above in the 1^{st} specific embodiment.

In a 4^{th} specific embodiment, for formula (C1′); Rₐ and R_{b} are both H; and R₅ is H or Me; and the remaining variables are as described above in the 1^{st} or 2^{nd} specific embodiment.

In a 5^{th} specific embodiment, for formula (C1′), P is a peptide containing 2 to 5 amino acid residues; and the remaining variables are as described above in the 1^{st}, 2^{nd} or 4^{th} specific embodiment. In a more specific embodiment, P is selected from Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), β-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. More specifically, P is Gly-Gly-Gly, Ala-Val, Ala-Ala, Ala-D-Ala, D-Ala-Ala, or D-Ala-D-Ala.

In a 6^{th} specific embodiment, for formula (C1′), Q is —SO₃M; and the remaining variables are as describe above in the 1^{st}, 2^{nd}, 4^{th} or 5^{th} specific embodiment or any more specific embodiments described therein.

In a 7^{h} specific embodiment, for formula (C1′) or (C2′), R₁₉ and R₂₀ are both H; and m″ is an integer from 1 to 6; and the remaining variables are as described above in the 1^{st}, 2^{nd}, 3^{rd}, 4^{th}, 5^{th} or 6^{th} specific embodiment or any more specific embodiments described therein.

In a 8^{th} specific embodiment, for formula (C1′) or (C2′), -L_{C}^{Cys1} is represented by the following formula:

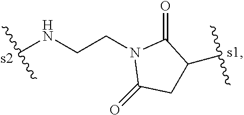 [SMILES: *NCCN1C(=O)CC(*)C1=O]

and the remaining variables are as described above in the 1^{st}, 2^{nd}, 3^{rd}, 4^{th}, 5^{th}, 6^{th} or 7^{th} specific embodiment or any more specific embodiments described therein.

In a 9^{th} specific embodiment, the conjugate of the third embodiment is represented by the following formula:

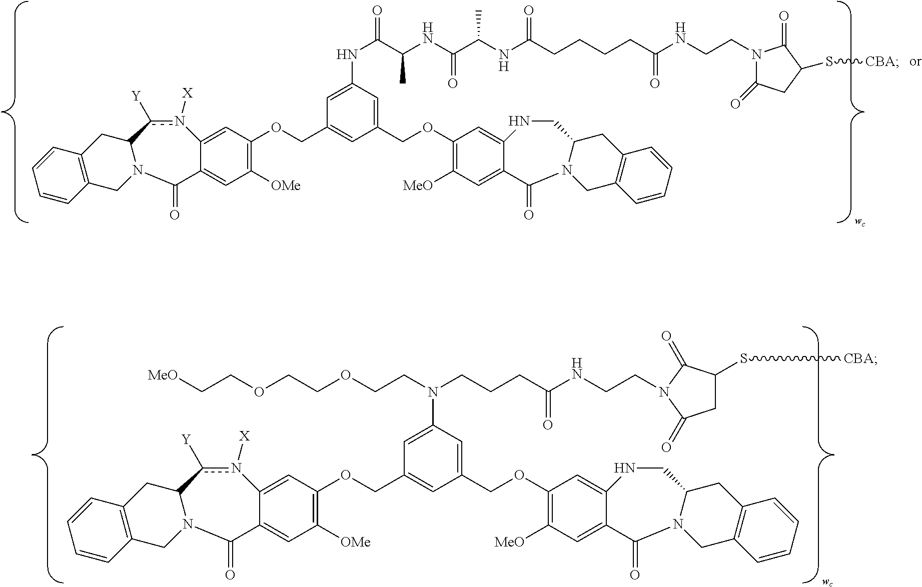

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃M. In a more specific embodiment, the double line [symbol] between N and C represents a double bond, X is absent and Y is —H. In another more specific embodiment, the double line [symbol] between N and C represents a single bond, X is —H and Y is —SO₃M.

In a 10^{th} specific embodiment, L^{Cys1} is represented by formula (C3′) or (C4′), and the remaceuticayining variabl e as described in the 1^{st} specific embodiment.

In a more specific embodiment, q and r are each independently an integer between 1 to 6, more specifically, an integer between 1 to 3. Even more specifically, R₁₀, R₁₁, R₁₂ and R₁₃ are all H.

In another more specific embodiment, m and n are each independently an integer between 1 and 6, more specifically, an integer between 1 to 3. Even more specifically, R₁₉, R₂₀, R₂₁ and R₂₂ are all H.

In a 11^{th} specific embodiment, L^{Cys1} is represented by formula (C3′); and the remaining variables are as described above in the 10^{th} specific embodiment or any more specific embodiments described therein.

In a 12^{th} specific embodiment, L^{Cys1} is represented by formula (C4′); and the remaining variables are as described above in the 10^{th} specific embodiment.

In a 13^{th} specific embodiment, for formulae (C3′) and (C₄′), P′ is a peptide containing 2 to 5 amino acid residues; and the remaining variables are as described in the 10^{th}, 11^{th} or 12^{th} specific embodiment or any more specific embodiments described therein. In a more specific embodiment, P′ is selected from Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), f3-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala. Even more specifically, P is Gly-Gly-Gly, Ala-Val, Ala-Ala, Ala-D-Ala, D-Ala-Ala, or D-Ala-D-Ala.

In a 14^{th} specific embodiment, for formula (C3′) or (C4′), -L_{C1}^{Cys1} is represented by the following formula:

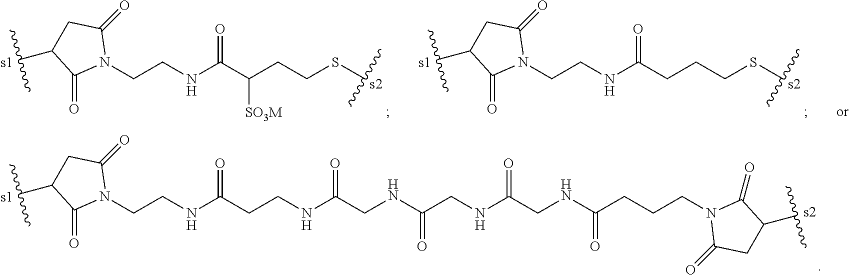

In a 15^{th} specific embodiment, for formula (C3′) or (C4′), R^{e} is H or Me; R^{x1} is —(CH₂)ₚ—(CR^{f}R^{g})—, and R^{x2} is —(CH₂)ₚ—(CR^{f}R^{g})—, wherein R^{f} and R^{g} are each independently —H or a (C₁-C₄)alkyl; and p is 0, 1, 2 or 3; and the remaining variables are as described above in the 10^{th}, 11^{th}, 12^{th}, 13^{th}, or 14^{th} specific embodiment. More specifically, R^{f} and R^{g} are the same or different, and are selected from —H and -Me.

In a 16^{th} specific embodiment, the conjugate of the third embodiment is represented by the following formula:

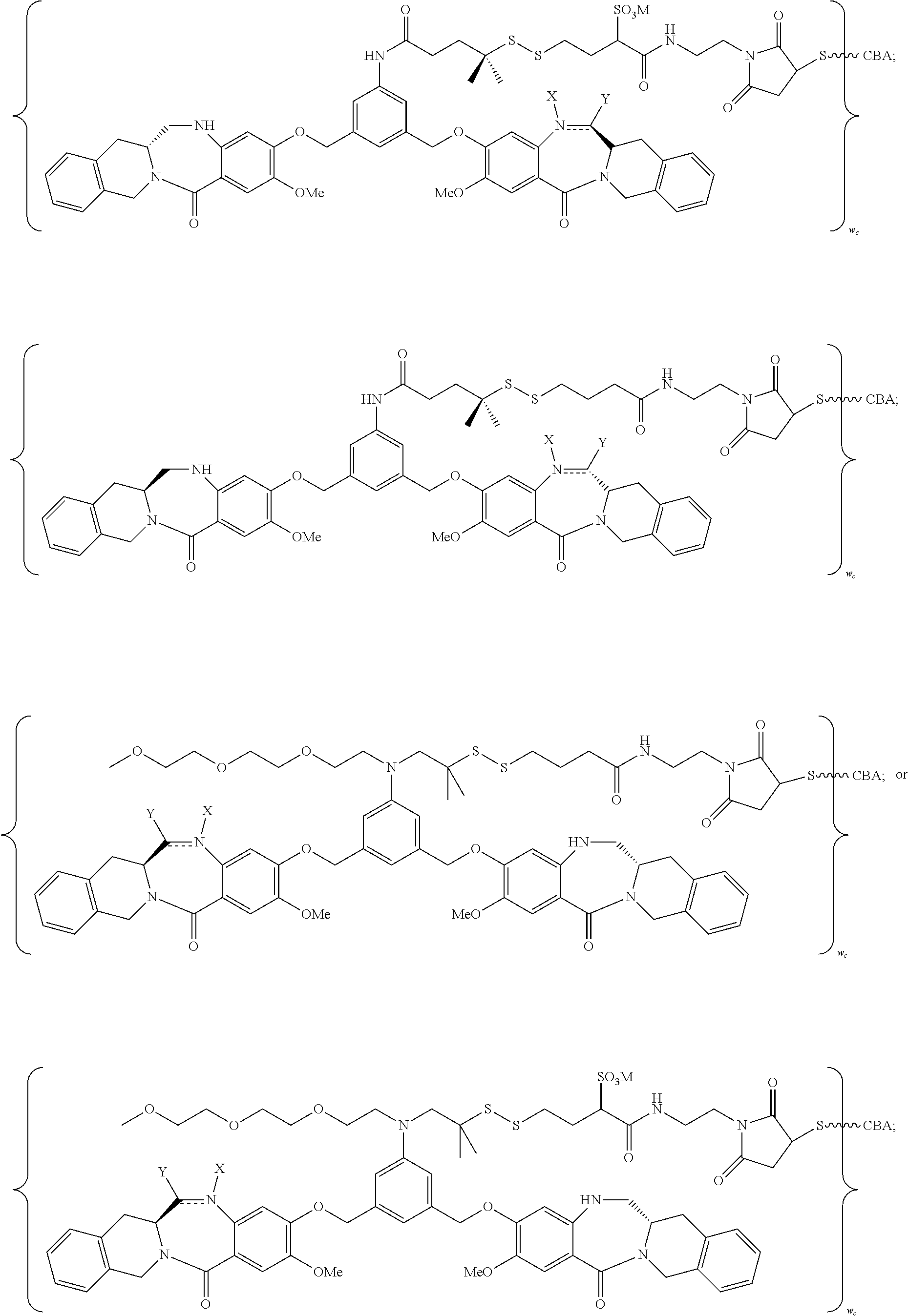

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO₃M. In a more specific embodiment, the double line [symbol] between N and C represents a double bond, X is absent and Y is —H. In another specific embodiment, the double line [symbol] between N and C represents a single bond, X is —H and Y is —SO₃M.

In some embodiments, the CBA comprises the subject antibody or antigen-binding fragment thereof, has a Cys residue at a location corresponding to the engineered Cys in the heavy chain CH3 domain.

In another embodiment, the conjugates of the third embodiment described above can be prepared by reacting the CBA having one or more free cysteine with a cytotoxic agent having a thiol-reactive group described herein.

Cell-Binding Agents

The effectiveness of the conjugates of the invention as therapeutic agents depends on the careful selection of an appropriate cell-binding agent. Cell-binding agents can be of any kind presently known, or that become known, including peptides and non-peptides. Generally, these can be antibodies (such as polyclonal antibodies and monoclonal antibodies, especially monoclonal antibodies), lymphokines, hormones, growth factors, vitamins (such as folate etc., which can bind to a cell surface receptor thereof, e.g., a folate receptor), nutrient-transport molecules (such as transferrin), or any other cell-binding molecule or substance.

Selection of the appropriate cell-binding agent is a matter of choice that partly depends upon the particular cell population that is to be targeted, but in many (but not all) cases, human monoclonal antibodies are a good choice if an appropriate one is available. For example, the monoclonal antibody MY9 is a murine IgG₁ antibody that binds specifically to the CD33 Antigen (J. D. Griffin et al., Leukemia Res., 8:521 (1984)), and can be used if the target cells express CD33 as in the disease of acute myelogenous leukemia (AML).

In certain embodiments, the cell-binding agent is not a protein. For example, in certain embodiments, the cell binding agent may be a vitamin that binds to a vitamin receptor, such as a cell-surface receptor. In this regard, vitamin A binds to retinol-binding protein (RBP) to form a complex, which complex in turn binds the STRA6 receptor with high affinity and increases vitamin A in-take. In another example, folic acid/folate/vitamin B₉ binds the cell-surface folate receptor (FR), for example, FRa, with high affinity. Folic acid or antibodies that bind to FRa can be used to target the folate receptor expressed on ovarian and other tumors. In addition, vitamin D and its analog bind to vitamin D receptor.

In other embodiments, the cell-binding agent is a protein or a polypeptide, or a compound comprising a protein or polypeptide, including antibody, non-antibody protein, or polypeptide. Preferably, the protein or polypeptides comprise one or more Lys residues with side chain —NH₂ group. The Lys side chain —NH₂ groups can be covalently linked to the bifunctional crosslinkers, which in turn are linked to the dimer compounds of the invention, thus conjugating the cell-binding agents to the dimer compounds of the invention. Each protein-based cell-binding agents can contain multiple Lys side chain —NH₂ groups available for linking the compounds of the invention through the bifunctional crosslinkers.

In some embodiments, GM-CSF, a ligand/growth factor which binds to myeloid cells can be used as a cell-binding agent to diseased cells from acute myelogenous leukemia. IL-2 which binds to activated T-cells can be used for prevention of transplant graft rejection, for therapy and prevention of graft-versus-host disease, and for treatment of acute T-cell leukemia. MSH, which binds to melanocytes, can be used for the treatment of melanoma, as can antibodies directed towards melanomas. Epidermal growth factor can be used to target squamous cancers, such as lung and head and neck.

Somatostatin can be used to target neuroblastomas and other tumor types. Estrogen (or estrogen analogues) can be used to target breast cancer. Androgen (or androgen analogues) can be used to target testes.

In certain embodiments, the cell-binding agent can be a lymphokine, a hormone, a growth factor, a colony stimulating factor, or a nutrient-transport molecule.

In certain embodiments, the cell-binding agent is an antibody mimetic, such as an ankyrin repeat protein, a Centyrin, or an adnectin/monobody.

In other embodiments, the cell-binding agent is an antibody, a single chain antibody, an antibody fragment that specifically binds to the target cell, a monoclonal antibody, a single chain monoclonal antibody, a monoclonal antibody fragment (or “antigen-binding portion”) that specifically binds to a target cell, a chimeric antibody, a chimeric antibody fragment (or “antigen-binding portion”) that specifically binds to the target cell, a domain antibody (e.g., sdAb), or a domain antibody fragment that specifically binds to the target cell.

In certain embodiments, the cell-binding agent is a humanized antibody, a humanized single chain antibody, or a humanized antibody fragment (or “antigen-binding portion”). In a specific embodiment, the humanized antibody is huMy9-6 or another related antibody, which is described in U.S. Pat. Nos. 7,342,110 and 7,557,189. In another specific embodiment, the humanized antibody is an anti-folate receptor antibody described in U.S. Provisional Application Nos. 61/307,797, 61/346,595, and 61/413,172 and U.S. application Ser. No. 13/033,723 (published as US 2012/0009181 A1). The teachings of all these applications are incorporated herein by reference in its entirety.

In certain embodiments, the cell-binding agent is a resurfaced antibody, a resurfaced single chain antibody, a resurfaced antibody fragment (or “antigen-binding portion”), or a bispecific antibody.

In certain embodiments, the cell-binding agent is a minibody, an avibody, a diabody, a tribody, a tetrabody, a nanobody, a probody, a domain antibody, or an unibody.

In other words, an exemplary cell binding agent may include an antibody, a single chain antibody, an antibody fragment that specifically binds to the target cell, a monoclonal antibody, a single chain monoclonal antibody, a monoclonal antibody fragment that specifically binds to a target cell, a chimeric antibody, a chimeric antibody fragment that specifically binds to the target cell, a bispecific antibody, a domain antibody, a domain antibody fragment that specifically binds to the target cell, an interferon (e.g., α, β, γ), a lymphokine (e.g., IL-2, IL-3, IL-4, and IL-6), a hormone (e.g., insulin, thyrotropin releasing hormone (TRH), melanocyte-stimulating hormone (MSH), and a steroid hormone (e.g., androgen and estrogen)), a vitamin (e.g., folate), a growth factor (e.g., EGF, TGF-alpha, FGF, VEGF), a colony stimulating factor, a nutrient-transport molecule (e.g., transferrin; see O'Keefe et al. (1985) J. Biol. Chem. 260:932-937, incorporated herein by reference), a Centyrin (a protein scaffold based on a consensus sequence of fibronectin type III (FN3) repeats; see U.S. Patent Publication 2010/0255056, 2010/0216708 and 2011/0274623 incorporated herein by reference), an Ankyrin Repeat Protein (e.g., a designed ankyrin repeat protein, known as DARPin; see U.S. Patent Publication Nos. 2004/0132028, 2009/0082274, 2011/0118146, and 2011/0224100, incorporated herein by reference, and also see C. Zahnd et al., Cancer Res. (2010) 70:1595-1605; Zahnd et al., J. Biol. Chem. (2006) 281(46):35167-35175; and Binz, H. K., Amstutz, P. & Pluckthun, A., Nature Biotechnology (2005) 23:1257-1268, incorporated herein by reference), an ankyrin-like repeats protein or synthetic peptide (see e.g., U.S. Patent Publication No. 2007/0238667; U.S. Pat. No. 7,101,675; WO 2007/147213; and WO 2007/062466, incorporated herein by reference), an Adnectin (a fibronectin domain scaffold protein; see US Patent Publication Nos. 2007/0082365; 2008/0139791, incorporated herein by reference), Avibody (including diabodies, triabodies, and tetrabodies; see U.S. Publication Nos. 2008/0152586 and 2012/0171115), dual receptor retargeting (DART) molecules (P. A. Moore et al., Blood, 2011; 117(17):4542-4551; Veri M C, et al., Arthritis Rheum, 2010 Mar. 30; 62(7):1933-43; Johnson S, et al., J. Mol. Biol., 2010 Apr. 9; 399(3):436-49), cell penetrating supercharged proteins (Methods in Enzymol. 502, 293-319 (2012), and other cell-binding molecules or substances.

In certain embodiments, the cell-binding agent may be a ligand that binds to a moiety on the target cell, such as a cell-surface receptor. For example, the ligand may be a growth factor or a fragment thereof that binds to a growth factor receptor; or may be a cytokine or a fragment thereof that binds to a cytokine receptor. In certain embodiments, the growth factor receptor or cytokine receptor is a cell-surface receptor.

In certain embodiments, wherein the cell-binding agent is an antibody or an antigen-binding portion thereof (including antibody derivatives), or certain antibody mimetics, the CBA may bind to a ligand on the target cell, such as a cell-surface ligand, including cell-surface receptors.

Specific exemplary antigens or ligands may include renin; a growth hormone (e.g., human growth hormone and bovine growth hormone); a growth hormone releasing factor; a parathyroid hormone; a thyroid stimulating hormone; a lipoprotein; alpha-1-antitrypsin; insulin A-chain; insulin B-chain; proinsulin; a follicle stimulating hormone; calcitonin; a luteinizing hormone; glucagon; a clotting factor (e.g., factor vmc, factor IX, tissue factor, and von Willebrands factor); an anti-clotting factor (e.g., Protein C); an atrial natriuretic factor; a lung surfactant; a plasminogen activator (e.g., a urokinase, a human urine or tissue-type plasminogen activator); bombesin; a thrombin; hemopoietic growth factor; tumor necrosis factor-alpha and -beta; an enkephalinase; RANTES (i.e., the regulated on activation normally T-cell expressed and secreted); human macrophage inflammatory protein-1-alpha; a serum albumin (human serum albumin); Muellerian-inhibiting substance; relaxin A-chain; relaxin B-chain; prorelaxin; a mouse gonadotropin-associated peptide; a microbial protein (beta-lactamase); DNase; IgE; a cytotoxic T-lymphocyte associated antigen (e.g., CTLA-4); inhibin; activin; a vascular endothelial growth factor; a receptor for hormones or growth factors; protein A or D; a rheumatoid factor; a neurotrophic factor (e.g., bone-derived neurotrophic factor, neurotrophin-3, -4, -5, or -6), a nerve growth factor (e.g., NGF-β); a platelet-derived growth factor; a fibroblast growth factor (e.g., aFGF and bFGF); fibroblast growth factor receptor 2; an epidermal growth factor; a transforming growth factor (e.g., TGF-alpha, TGF-β1, TGF-β2, TGF-β3, TGF-β4, and TGF-β5); insulin-like growth factor-I and —II; des(1-3)-IGF-I (brain IGF-I); an insulin-like growth factor binding protein; melanotransferrin; CA6, CAK1, CALLA, CAECAM5, EpCAM; GD3; FLT3; PSMA; PSCA; MUC1; MUC16; STEAP; CEA; TENB2; an EphA receptor; an EphB receptor; a folate receptor; FOLR1; mesothelin; cripto; an alphaᵥbeta₆; integrins; VEGF; VEGFR; EGFR; FGFR3; LAMP1, p-cadherin, transferrin receptor; IRTA1; IRTA2; IRTA3; IRTA4; IRTA5; CD proteins (e.g., CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD123, CD134, CD137, CD138, and CD152), one or more tumor-associated antigens or cell-surface receptors (see US Publication No. 2008/0171040 or US Publication No. 2008/0305044, incorporated in their entirety by reference); erythropoietin; an osteoinductive factor; an immunotoxin; a bone morphogenetic protein; an interferon (e.g., interferon-alpha, -beta, and -gamma); a colony stimulating factor (e.g., M-CSF, GM-CSF, and G-CSF); interleukins (e.g., IL-1 to IL-10); a superoxide dismutase; a T-cell receptor; a surface membrane protein; a decay accelerating factor; a viral antigen s (e.g., a portion of the HIV envelope); a transport protein, a homing receptor; an addressin; a regulatory protein; an integrin (e.g., CD11a, CD11b, CD11c, CD18, an ICAM, VLA-4, and VCAM;) a tumor associated antigen (e.g., HER2, HER3 and HER4 receptor); endoglin; c-Met; c-kit; 1GF1R; PSGR; NGEP; PSMA; PSCA; TMEFF2; LGR5; B7H₄; and fragments of any of the above-listed polypeptides.

As used herein, the term “antibody” includes immunoglobulin (Ig) molecules. In certain embodiments, the antibody is a full-length antibody that comprises four polypeptide chains, namely two heavy chains (HC) and two light chains (LC) inter-connected by disulfide bonds. Each heavy chain is comprised of a heavy chain variable region (HCVR or VH) and a heavy chain constant region (CH). The heavy chain constant region is comprised of three domains, CH1, CH2, and CH3. Each light chain is comprised of a light chain variable region (LCVR or VL) and a light chain constant region, which is comprised of one domain, CL. The VH and VL regions can be further subdivided into regions of hypervariability, termed complementarity determining regions (CDRs). Interspersed with such regions are the more conserved framework regions (FRs). Each VH and VL is composed of three CDRs and four FR₅, arranged from amino-terminus to carboxy-terminus in the following order: FR1, CDR1, FR2, CDR2, FR3, CDR3, and FR4.

In certain embodiments, the antibody is IgG, IgA, IgE, IgD, or IgM. In certain embodiments, the antibody is IgG1, IgG2, IgG3, or IgG4; or IgA1 or IgA2.

In certain embodiments, the cell-binding agent is an “antigen-binding portion” of a monoclonal antibody, sharing sequences critical for antigen-binding with an antibody (such as huMy9-6 or its related antibodies described in U.S. Pat. Nos. 7,342,110 and 7,557,189, incorporated herein by reference).

As used herein, the term “antigen-binding portion” of an antibody (or sometimes interchangeably referred to as “antibody fragments”), include one or more fragments of an antibody that retain the ability to specifically bind to an antigen. It has been shown that the antigen-binding function of an antibody can be performed by certain fragments of a full-length antibody. Examples of binding fragments encompassed within the term “antigen-binding portion” of an antibody include (without limitation): (i) a Fab fragment, a monovalent fragment consisting of the VL, VH, CL and CH1 domains (e.g., an antibody digested by papain yields three fragments: two antigen-binding Fab fragments, and one Fc fragment that does not bind antigen); (ii) a F(ab′)₂ fragment, a bivalent fragment comprising two Fab fragments linked by a disulfide bridge at the hinge region (e.g., an antibody digested by pepsin yields two fragments: a bivalent antigen-binding F(ab′)₂ fragment, and a pFc′ fragment that does not bind antigen) and its related F(ab′) monovalent unit; (iii) a Fd fragment consisting of the VH and CH1 domains (i.e., that portion of the heavy chain which is included in the Fab); (iv) a Fv fragment consisting of the VL and VH domains of a single arm of an antibody, and the related disulfide linked Fv; (v) a dAb (domain antibody) or sdAb (single domain antibody) fragment (Ward et al., Nature 341:544-546, 1989), which consists of a VH domain; and (vi) an isolated complementarity determining region (CDR). In certain embodiments, the antigen-binding portion is a sdAb (single domain antibody).

In certain embodiments, antigen-binding portion also include certain engineered or recombinant derivatives (or “derivative antibodies”) that also include one or more fragments of an antibody that retain the ability to specifically bind to an antigen, in addition to elements or sequences that may not be found in naturally existing antibodies.

For example, although the two domains of the Fv fragment, VL and VH, are coded for by separate genes, they can be joined, using standard recombinant methods, by a synthetic linker that enables them to be made as a single protein chain in which the VL and VH regions pair to form monovalent molecules (known as single chain Fv (scFv); see, e.g., Bird et al. Science 242:423-426, 1988: and Huston et al., Proc. Natl. Acad. Sci. USA 85:5879-5883, 1988).

In all embodiments described herein, the N-terminum of an scFv may be a VH domain (i.e., N—VH—VL-C), or a VL domain (i.e., N-VL-VH-C).

Divalent (or bivalent) single-chain variable fragments (di-scFvs, bi-scFvs) can be engineered by linking two scFvs. This produces a single peptide chain with two VH and two VL regions, yielding a tandem scFvs (tascFv). More tandem repeats, such as tri-scFv, may be similarly produced by linking three or more scFv in a head-to-tail fashion.

In certain embodiments, scFvs may be linked through linker peptides that are too short (about five amino acids) for the two variable regions to fold together, forcing scFvs to dimerize, and form diabodies (see, e.g., Holliger et al., Proc. Natl. Acad. Sci. USA 90:6444-6448, 1993; Poljak et al., Structure 2:1121-1123, 1994). Diabodies may be bi-specific or monospecific. Diabodies have been shown to have dissociation constants up to 40-fold lower than corresponding scFvs, i.e., having a much higher affinity to the target.

Still shorter linkers (one or two amino acids) lead to the formation of trimers, or so-called triabodies or tribodies. Tetrabodies have also been produced similarly. They exhibit an even higher affinity to their targets than diabodies. Diabodies, triabodies, and tetrabodies are sometimes collectively called “AVIBODY™” cell binding agents (or “AVIBODY” in short). That is, AVIBODY having two, three, or four Target Binding Regions (TBRs) are commonly known as Dia-, Tria- and Tetrabodies. See, for example, U.S. Publication Nos. 2008/0152586 and 2012/0171115 for details, the entire teachings of which are incorporated herein by reference.

All of these formats can be composed from variable fragments with specificity for two or more different antigens, in which case they are types of bi- or multi-specific antibodies. For example, certain bispecific tandem di-scFvs, are known as bi-specific T-cell engagers (BiTEs).

In certain embodiments, each scFv in the tandem scFv or diabody/triabody/tetrabody may have the same or different binding specificity, and each may independently have an N-terminal VH or N-terminal VL.

Single chain Fv (scFv) can also be fused to an Fc moiety, such as the human IgG Fc moiety to obtain IgG-like properties, but nevertheless they are still encoded by a single gene. As transient production of such scFv-Fc proteins in mammalians can easily achieve milligram amounts, this derivative antibody format is particularly suitable for many research applications.

Fcabs are antibody fragments engineered from the Fc constant region of an antibody. Fcabs can be expressed as soluble proteins, or they can be engineered back into a full-length antibody, such as IgG, to create mAb2. A mAb2 is a full-length antibody with an Fcab in place of the normal Fc region. With these additional binding sites, mAb2 bispecific monoclonal antibodies can bind two different targets at the same time.

In certain embodiments, the engineered antibody derivatives have reduced size of the antigen-binding Ig-derived recombinant proteins (“miniaturized” full-size mAbs), produced by removing domains deemed non-essential for function. One of the best examples is SMIPs.

A Small modular immunopharmaceutical, or SMIP, is an artificial protein largely built from parts of antibodies (immunoglobulins), and is intended for use as a pharmaceutical drug. SMIPs have similar biological half-life as antibodies, but are smaller than antibodies and hence may have better tissue penetration properties. SMIPs are single-chain proteins that comprise one binding region, one hinge region as a connector, and one effector domain. The binding region comprises a modified single-chain variable fragment (scFv), and the rest of the protein can be constructed from the Fc (such as CH2, and CH3 as the effector domain) and the hinge region of an antibody, such as IgG1. Genetically modified cells produce SMIPs as antibody-like dimers that are about 30% smaller than real antibodies.

Another example of such engineered miniaturized antibody is “unibody,” in which the hinge region has been removed from IgG4 molecules. IgG4 molecules are unstable and can exchange light-heavy chain heterodimers with one another. Deletion of the hinge region prevents heavy chain-heavy chain pairing entirely, leaving highly specific monovalent light/heavy heterodimers, while retaining the Fc region to ensure stability and half-life in vivo.

A single-domain antibody (sdAb, including but not limited to those called nanobody by Ablynx) is an antibody fragment consisting of a single monomeric variable antibody domain. Like a whole antibody, it is able to bind selectively to a specific antigen, but is much smaller due to its molecular weight of only 12-15 kDa. In certain embodiments, the single-domain antibody is engineered from heavy-chain antibodies (hcIgG). The first such sdAb was engineered based on an hcIgG found in camelids, called V_{H}H fragments. In certain embodiments, the single-domain antibody is engineered from IgNAR (“immunoglobulin new antigen receptor,” see below) using a V_{NAR} fragment. Cartilaginous fishes (such as shark) have such heavy-chain IgNAR antibodies. In certain embodiments, the sdAb is engineered by splitting the dimeric variable domains from common immunoglobulin G (IgG), such as those from humans or mice, into monomers. In certain embodiments, a nanobody is derived from a heavy chain variable domain. In certain embodiments, a nanobody is derived from light chain variable domain. In certain embodiments, the sdAb is obtained by screening libraries of single domain heavy chain sequences (e.g., human single domain HCs) for binders to a target antigen.

The single variable new antigen receptor domain antibody fragments (V_{NAR}S, or V_{NAR} domains) are derived from cartilaginous fish (e.g., shark) immunoglobulin new antigen receptor antibodies (IgNARs). Being one of the smallest known immunoglobulin-based protein scaffolds, such single domain proteins demonstrate favorable size and cryptic epitope recognition properties. Mature IgNAR antibodies consist of homodimers of one variable new antigen receptor (V_{NAR}) domain and five constant new antigen receptor (C_{NAR}) domains. This molecule is highly stable, and possesses efficient binding characteristics. Its inherent stability can likely be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

A minibody is an engineered antibody fragment comprising an scFv linked to a CH domain, such as the CH3γ1 (CH3 domain of IgG1) or CH4ε (CH4 domain of IgE). For example, an scFv specific for carcinoembryonic antigen (CEA) has been linked to the CH3γ1 to create a minibody, which has previously been demonstrated to possess excellent tumor targeting coupled with rapid clearance in vivo (Hu et al., Cancer Res. 56:3055-3061, 1996). The scFv may have a N-terminal VH or VL. The linkage may be a short peptide (e.g., two amino acid linker, such as ValGlu) that results in a non-covalent, hingeless minibody. Alternatively, the linkage may be an IgG1 hinge and a GlySer linker peptide that produces a covalent, hinge-minibody.

Natural antibodies are mono-specific, but bivalent, in that they express two identical antigen-binding domains. In contrast, in certain embodiments, certain engineered antibody derivatives are bi- or multi-specific molecules possess two or more different antigen-binding domains, each with different target specificity. Bispecific antibodies can be generated by fusing two antibody-producing cells, each with distinct specificity. These “quadromas” produced multiple molecular species, as the two distinct light chains and two distinct heavy chains were free to recombine in the quadromas in multiple configurations. Since then, bispecific Fabs, scFvs and full-size mAbs have been generated using a variety of technologies (see above).

The dual variable domain immunoglobulin (DVD-Ig) protein is a type of dual-specific IgG that simultaneously target two antigens/epitopes (DiGiammarino et al., Methods Mol. Biol., 899:145-56, 2012). The molecule contains an Fc region and constant regions in a configuration similar to a conventional IgG. However, the DVD-Ig protein is unique in that each arm of the molecule contains two variable domains (VDs). The VDs within an arm are linked in tandem and can possess different binding specificities.

Trispecific antibody derivative molecules can also been generated by, for example, expressing bispecific antibodies with two distinct Fabs and an Fc. One example is a mouse IgG2a anti-Ep-CAM, rat IgG2b anti-CD3 quadroma, called BiUII, which is thought to permit the co-localization of tumor cells expressing Ep-CAM, T cells expressing CD3, and macrophages expressing FCyRI, thus potentiating the costimulatory and anti-tumor functions of the immune cells.

Probodies are fully recombinant, masked monoclonal antibodies that remain inert in healthy tissue, but are activated specifically in the disease microenvironment (e.g., through protease cleavage by a protease enriched or specific in a disease microenvironment). See Desnoyers et al., Sci. Transl. Med., 5:207ra144, 2013. Similar masking techniques can be used for any of the antibodies or antigen-binding portions thereof described herein.

An intrabody is an antibody that has been modified for intracellular localization, for working within the cell to bind to an intracellular antigen. The intrabody may remain in the cytoplasm, or may have a nuclear localization signal, or may have a KDEL (SEQ ID NO:33) sequence for ER targeting. The intrabody may be a single-chain antibody (scFv), modified immunoglobulin VL domains with hyperstability, selected antibody resistant to the more reducing intracellular environment, or expressed as a fusion protein with maltose binding protein or other stable intracellular proteins. Such optimizations have improved the stability and structure of intrabodies, and may have general applicability to any of the antibodies or antigen-binding portions thereof described herein.

The antigen-binding portions or derivative antibodies of the invention may have substantially the same or identical (1) light chain and/or heavy chain CDR3 regions; (2) light chain and/or heavy chain CDR1, CDR2, and CDR3 regions; or (3) light chain and/or heavy chain regions, compared to an antibody from which they are derived/engineered. Sequences within these regions may contain conservative amino acid substitutions, including substitutions within the CDR regions. In certain embodiments, there is no more than 1, 2, 3, 4, or 5 conservative substitutions. In an alternative, the antigen-binding portions or derivative antibodies have a light chain region and/or a heavy chain region that is at least about 90%, 95%, 99% or 100% identical to an antibody from which they are derived/engineered. These antigen-binding portions or derivative antibodies may have substantially the same binding specificity and/or affinity to the target antigen compared to the antibody. In certain embodiments, the K_{d} and/or k_{off} values of the antigen-binding portions or derivative antibodies are within 10-fold (either higher or lower), 5-fold (either higher or lower), 3-fold (either higher or lower), or 2-fold (either higher or lower) of an antibody described herein.

In certain embodiments, the antigen-binding portions or derivative antibodies may be derived/engineered from fully human antibodies, humanized antibodies, or chimeric antibodies, and may be produced according to any art-recognized methods.

Monoclonal antibody techniques allow for the production of extremely specific cell-binding agents in the form of specific monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Sensitized human cells can also be used. Another method of creating monoclonal antibodies is the use of phage libraries of scFv (single chain variable region), specifically human scFv (see e.g., Griffiths et al., U.S. Pat. Nos. 5,885,793 and 5,969,108; McCafferty et al., WO 92/01047; Liming et al., WO 99/06587). In addition, resurfaced antibodies disclosed in U.S. Pat. No. 5,639,641 may also be used, as may chimeric antibodies and humanized antibodies.

Cell-binding agent can also be peptides derived from phage display (see, for example, Wang et al., Proc. Natl. Acad. Sci. USA (2011) 108(17), 6909-6914) or peptide library techniques (see, for example, Dane et al., Mol. Cancer. Ther. (2009) 8(5):1312-1318).

In certain embodiments, the CBA of the invention also includes an antibody mimetic, such as a DARPin, an affibody, an affilin, an affitin, an anticalin, an avimer, a Fynomer, a Kunitz domain peptide, a monobody, or a nanofitin.

As used herein, the terms “DARPin” and “(designed) ankyrin repeat protein” are used interchangeably to refer to certain genetically engineered antibody mimetic proteins typically exhibiting preferential (sometimes specific) target binding. The target may be protein, carbohydrate, or other chemical entities, and the binding affinity can be quite high. The DARPins may be derived from natural ankyrin repeat-containing proteins, and preferably consist of at least three, usually four or five ankyrin repeat motifs (typically about 33 residues in each ankyrin repeat motif) of these proteins. In certain embodiments, a DARPin contains about four- or five-repeats, and may have a molecular mass of about 14 or 18 kDa, respectively. Libraries of DARPins with randomized potential target interaction residues with diversities of over 10¹² variants can be generated at the DNA level, for use in selecting DARPins that bind desired targets (e.g., acting as receptor agonists or antagonists, inverse agonists, enzyme inhibitors, or simple target protein binders) with picomolar affinity and specificity, using a variety of technologies such as ribosome display or signal recognition particle (SRP) phage display. See, for example, U.S. Patent Publication Nos. 2004/0132028, 2009/0082274, 2011/0118146, and 2011/0224100, WO 02/20565 and WO 06/083275 for DARPin preparation (the entire teachings of which are incorporated herein by reference), and also see C. Zahnd et al. (2010) Cancer Res., 70:1595-1605; Zahnd et al. (2006) J. Biol. Chem., 281(46):35167-35175; and Binz, H. K., Amstutz, P. & Pluckthun, A. (2005) Nature Biotechnology, 23:1257-1268 (all incorporated herein by reference). Also see U.S. Patent Publication No. 2007/0238667; U.S. Pat. No. 7,101,675; WO 2007/147213; and WO 2007/062466 (the entire teachings of which are incorporated herein by reference), for the related ankyrin-like repeats protein or synthetic peptide.

Affibody molecules are small proteins engineered to bind to a large number of target proteins or peptides with high affinity, thus imitating monoclonal antibodies. An Affibody consists of three alpha helices with 58 amino acids and has a molar mass of about 6 kDa. They have been shown to withstand high temperatures (90° C.) or acidic and alkaline conditions (pH 2.5 or pH 11), and binders with an affinity of down to sub-nanomolar range have been obtained from naïve library selections, and binders with picomolar affinity have been obtained following affinity maturation. In certain embodiments, affibodies are conjugated to weak electrophiles for binding to targets covalently.

Monobodies (also known as Adnectins), are genetically engineered antibody mimetic proteins capable of binding to antigens. In certain embodiments, monobodies consist of 94 amino acids and have a molecular mass of about 10 kDa. They are based on the structure of human fibronectin, more specifically on its tenth extracellular type III domain, which has a structure similar to antibody variable domains, with seven beta sheets forming a barrel and three exposed loops on each side corresponding to the three complementarity determining regions. Monobodies with specificity for different proteins can be tailored by modifying the loops BC (between the second and third beta sheets) and FG (between the sixth and seventh sheets).

A tribody is a self-assembly antibody mimetic designed based on the C-terminal coiled-coil region of mouse and human cartilage matrix protein (CMP), which self-assembles into a parallel trimeric complex. It is a highly stable trimeric targeting ligand created by fusing a specific target-binding moiety with the trimerization domain derived from CMP. The resulting fusion proteins can efficiently self-assemble into a well-defined parallel homotrimer with high stability. Surface plasmon resonance (SPR) analysis of the trimeric targeting ligands demonstrated significantly enhanced target-binding strength compared with the corresponding monomers. Cellular-binding studies confirmed that such tribodies have superior binding strength toward their respective receptors.

A Centyrin is another antibody mimetic that can be obtained using a library built upon the framework of a consensus FN3 domain sequence (Diem et al., Protein Eng. Des. Sel., 2014). This library employs diversified positions within the C-strand, CD-loop, F-strand and FG-loop of the FN3 domain, and high-affinity Centyrin variants can be selected against specific targets.

In some embodiments, the cell-binding agent is an anti-folate receptor antibody.

More specifically, the anti-folate receptor antibody is a humanized antibody or antigen binding fragment thereof that specifically binds a human folate receptor 1 (also known as folate receptor alpha (FR-α)). The terms “human folate receptor 1,” “FOLR1,” or “folate receptor alpha (FR-α)”, as used herein, refers to any native human FOLR1, unless otherwise indicated. Thus, all of these terms can refer to either a protein or nucleic acid sequence as indicated herein. The term “FOLR1” encompasses “full-length,” unprocessed FOLR1 as well as any form of FOLR1 that results from processing within the cell. The FOLR1 antibody comprises: (a) a heavy chain CDR1 comprising GYFMN (SEQ ID NO: 4); a heavy chain CDR2 comprising RIHPYDGDTFYNQXaa₁FXaa₂Xaa₃ (SEQ ID NO: 5); and a heavy chain CDR3 comprising YDGSRAMDY (SEQ ID NO: 6); and (b) a light chain CDR1 comprising KASQSVSFAGTSLMH (SEQ ID NO: 7); a light chain CDR2 comprising RASNLEA (SEQ ID NO: 8); and a light chain CDR3 comprising QQSREYPYT (SEQ ID NO: 9); wherein Xaa₁ is selected from K, Q, H, and R; Xaa₂ is selected from Q, H, N, and R; and Xaa₃ is selected from G, E, T, S, A, and V. Preferably, the heavy chain CDR2 sequence comprises RIHPYDGDTFYNQKFQG (SEQ ID NO: 10).

In another embodiment, the anti-folate receptor antibody is a humanized antibody or antigen binding fragment thereof that specifically binds the human folate receptor 1 comprising the heavy chain having the amino acid sequence of

(SEQ ID NO: 11)

QVQLVQSGAEVVKPGASVKISCKASGYTFTGYFMNWVKQSPGQSLEWIGR
IHPYDGDTFYNQKFQGKATLTVDKSSNTAHMELLSLTSEDFAVYYCTRYD
GSRAMDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK.

In another embodiment, the anti-folate antibody receptor is a humanized antibody or antigen binding fragment thereof encoded by the plasmid DNA deposited with the ATCC on Apr. 7, 2010 and having ATCC deposit nos. PTA-10772 and PTA-10773 or 10774.

In another embodiment, the anti-folate receptor antibody is a humanized antibody or antigen binding fragment thereof that specifically binds the human folate receptor 1 comprising the light chain having the amino acid sequence of

(SEQ ID NO: 12)

DIVLTQSPLSLAVSLGQPAIISCKASQSVSFAGTSLMHWYHQKPGQQPRL
LIYRASNLEAGVPDRFSGSGSKTDFTLNISPVEAEDAATYYCQQSREYPY
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;
or

(SEQ ID NO: 13)

DIVLTQSPLSLAVSLGQPAIISCKASQSVSFAGTSLMHWYHQKPGQQPRL
LIYRASNLEAGVPDRFSGSGSKTDFTLTISPVEAEDAATYYCQQSREYPY
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In another embodiment the anti-folate receptor antibody is a humanized antibody or antigen binding fragment thereof that specifically binds the human folate receptor 1 comprising the heavy chain having the amino acid sequence of SEQ ID NO: 11, and the light chain having the amino acid sequence of SEQ ID NO: 12 or SEQ ID NO: 13. Preferably, the antibody comprises the heavy chain having the amino acid sequence of SEQ ID NO: 11 and the light chain having the amino acid sequence of SEQ ID NO: 13 (hu FOLR1).

In another embodiment, the anti-folate receptor antibody is a humanized antibody or antigen binding fragment thereof encoded by the plasmid DNA deposited with the ATCC on Apr. 7, 2010 and having ATCC deposit nos. PTA-10772 and PTA-10773 or 10774.

In another embodiment, the anti-folate receptor antibody is a humanized antibody or antigen binding fragment thereof that specifically binds the human folate receptor 1, and comprising a heavy chain variable domain at least about 90%, 95%, 99% or 100% identical to

(SEQ ID NO: 14)

QVQLVQSGAEVVKPGASVKISCKASGYTFTGYFMNWVKQSPGQSLEWIGR
IHPYDGDTFYNQKFQGKATLTVDKSSNTAHMELLSLTSEDFAVYYCTRYD
GSRAMDYWGQGTTVTVSS,

and a light chain variable domain at least about 90%, 95%, 99% or 100% identical to

(SEQ ID NO: 15)

DIVLTQSPLSLAVSLGQPAIISCKASQSVSFAGTSLMHWYHQKPGQQPRL
LIYRASNLEAGVPDRFSGSGSKTDFTLNISPVEAEDAATYYCQQSREYPY
TFGGGTKLEIKR;
or

(SEQ ID NO: 16)

DIVLTQSPLSLAVSLGQPAIISCKASQSVSFAGTSLMHWYHQKPGQQPRL
LIYRASNLEAGVPDRFSGSGSKTDFTLTISPVEAEDAATYYCQQSREYPY
TFGGGTKLEIKR.

In another embodiment, the anti-folate receptor antibody is huMov 19 or M9346A (see, for example, U.S. Pat. No. 8,709,432, U.S. Pat. No. 8,557,966, and WO2011106528, all incorporated herein by reference).

In another embodiment, the cell-binding agent is an anti-EGFR antibody or an antibody fragment thereof. In some embodiments, the anti-EGFR antibody is a non-antagonist antibody, including, for example, the antibodies described in WO2012058592, herein incorporated by reference. In another embodiment, the anti-EGFR antibody is a non-functional antibody, for example, humanized ML66 or EGFR-8. More specifically, the anti-EGFR antibody is huML66.

In yet another embodiment, the anti-EGFR antibody comprising the heavy chain having the amino acid sequence of SEQ ID NO: 17, and the light chain having the amino acid sequence of SEQ ID NO: 18. As used herein, double underlined sequences represent the variable regions (i.e., heavy chain variable region or HCVR, and light chain variable region or LCVR) of the heavy or light chain sequences, while bold sequences represent the CDR regions (i.e., from N-terminal to C-terminal, CDR1, CDR2, and CDR3, respectively, of the heavy chain or light chain sequences).

| Antibody | Full-Length Heavy/Light Chain Amino Acid Sequence |
|---|---|
| huML66HC | QVQLQESGPGLVKPSETLSLTCTVSGLSLASNSVSWIRQPPGKGLEWMGVIWNHG |
| | GTDYNPSIKSRLSISRDTSKSQVFLKMNSLTAADTAMYFCVRKGGIYFDYWGQGV |
| | LVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PG (SEQ ID NO: 17) |
| huML66LC | DTVLTQSPSLAVSPGERATISCRASESVSTLMHWYQQKPGQQPKLLIYLASHRESG |
| | VPARFSGSGSGTDFTLTIDPMEAEDTATYYCQQSRNDPWTFGQGTKLELKRTVAA |
| | PSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 18) |

In yet another embodiment, the anti-EGFR antibody comprises the heavy chain CDR1-CDR3 of SEQ ID NO: 17, and/or the light chain CDR1-CDR3 of SEQ ID NO: 18, and preferably specifically binds EGFR.

In yet another embodiment, the anti-EGFR antibody comprises a heavy chain variable region (HCVR) sequence at least about 90%, 95%, 97%, 99%, or 100% identical to SEQ ID NO: 17, and/or a light chain variable region (LCVR) sequence at least about 90%, 95%, 97%, 99%, or 100% identical to SEQ ID NO: 18, and preferably specifically binds EGFR.

In another embodiment, the anti-EGFR antibody are antibodies described in U.S. Pat. No. 8,790,649 and WO 2012/058588, herein incorporated by reference. In some embodiments, the anti-EGFR antibody is huEGFR-7R antiboby.

In some embodiments, the anti-EGFR antibody comprises an immunoglobulin heavy chain region having the amino acid sequence of

(SEQ ID NO: 19)

QVQLVQSGAEVAKPGASVKLSCKASGYTFTSYWMQWVKQRPGQGLECIGT
IYPGDGDTTYTQKFQGKATLTADKSSSTAYMQLSSLRSEDSAVYYCARYD
APGYAMDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG

and an immunoglobulin light chain region having the amino acid sequence of

(SEQ ID NO: 20)

DIQMTQSPSSLSASVGDRVTITCRASQDINNYLAWYQHKPGKGPKLLIHY
TSTLHPGIPSRFSGSGSGRDYSFSISSLEPEDIATYYCLQYDNLLYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,

or an immunoglobulin light chain region having the amino acid sequence of

(SEQ ID NO: 21)

DIQMTQSPSSLSASVGDRVTITCKASQDINNYLAWYQHKPGKGPKLLIHY
TSTLHPGIPSRFSGSGSGRDYSFSISSLEPEDIATYYCLQYDNLLYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In another embodiment, the anti-EGFR antibody comprises an immunoglobulin heavy chain region having the amino acid sequence set forth in SEQ ID NO: 19 and an immunoglobulin light chain region having the amino acid sequence set forth in SEQ ID NO:20.

In another embodiment, the anti-EGFR antibody comprises an immunoglobulin heavy chain region having the amino acid sequence set forth in SEQ ID NO: 19 and an immunoglobulin light chain region having the amino acid sequence set forth in SEQ ID NO:21.

In yet another embodiment, the anti-EGFR antibody comprises the heavy chain CDR1-CDR3 of SEQ ID NO: 19, and/or the light chain CDR1-CDR3 of SEQ ID NO: 20 or 21, and preferably specifically binds EGFR.

In yet another embodiment, the anti-EGFR antibody comprises a heavy chain variable region (HCVR) sequence at least about 90%, 95%, 97%, 99%, or 100% identical to SEQ ID NO: 19, and/or a light chain variable region (LCVR) sequence at least about 90%, 95%, 97%, 99%, or 100% identical to SEQ ID NO: 20 or 21, and preferably specifically binds EGFR.

In another embodiment, the cell-binding agent is an anti-CD19 antibody, such as those described in U.S. Pat. No. 8,435,528 and WO2004/103272, herein incorporated by reference. In some embodiments, the anti-CD19 antibody comprises an immunoglobulin heavy chain region having the amino acid sequence of

(SEQ ID NO: 22)

QVQLVQPGAEVVKPGASVKLSCKTSGYTFTSNWMHWVKQAPGQGLEWIGE
IDPSDSYTNYNQNFQGKAKLTVDKSTSTAYMEVSSLRSDDTAVYYCARGS
NPYYYAMDYWGQGTSVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

and an immunoglobulin light chain region having the amino acid sequence of

(SEQ ID NO: 23)

EIVLTQSPAIMSASPGERVTMTCSASSGVNYMHWYQQKPGTSPRRWIYDT
SKLASGVPARFSGSGSGTDYSLTISSMEPEDAATYYCHQRGSYTFGGGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGEC.

In another embodiment, the anti-CD19 antibody is huB4 antibody.

In yet another embodiment, the anti-CD19 antibody comprises the heavy chain CDR1-CDR3 of SEQ ID NO: 22, and/or the light chain CDR1-CDR3 of SEQ ID NO: 23, and preferably specifically binds CD19.

In yet another embodiment, the anti-CD19 antibody comprises a heavy chain variable region (HCVR) sequence at least about 90%, 95%, 97%, 99%, or 100% identical to SEQ ID NO: 22, and/or a light chain variable region (LCVR) sequence at least about 90%, 95%, 97%, 99%, or 100% identical to SEQ ID NO: 23, and preferably specifically binds CD19.

In yet another embodiment, the cell-binding agent is an anti-Muc1 antibody, such as those described in U.S. Pat. No. 7,834,155, WO 2005/009369 and WO 2007/024222, herein incorporated by reference. In some embodiments, the anti-Muc1 antibody comprises an immunoglobulin heavy chain region having the amino acid sequence of

(SEQ ID NO: 24)

QAQLVQSGAEVVKPGASVKMSCKASGYTFTSYNMHWVKQTPGQGLEWIGY
IYPGNGATNYNQKFQGKATLTADTSSSTAYMQISSLTSEDSAVYFCARGD
SVPFAYWGQGTLVTVSAASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

and an immunoglobulin light chain region having the amino acid sequence of

(SEQ ID NO: 25)

EIVLTQSPATMSASPGERVTITCSAHSSVSFMHWFQQKPGTSPKLWIYST
SSLASGVPARFGGSGSGTSYSLTISSMEAEDAATYYCQQRSSFPLTFGAG
TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC.

In another embodiment, the anti-Muc1 antibody is huDS6 antibody.

In yet another embodiment, the anti-Muc1 antibody comprises the heavy chain CDR1-CDR3 of SEQ ID NO: 24, and/or the light chain CDR1-CDR3 of SEQ ID NO: 25, and preferably specifically binds Muc1.

In yet another embodiment, the anti-Muc1 antibody comprises a heavy chain variable region (HCVR) sequence at least about 90%, 95%, 97%, 99%, or 100% identical to SEQ ID NO: 24, and/or a light chain variable region (LCVR) sequence at least about 90%, 95%, 97%, 99%, or 100% identical to SEQ ID NO: 25, and preferably specifically binds Muc1.

In another embodiment, the cell-binding agent is an anti-CD33 antibody or fragment thereof, such as the antibodies or fragments thereof described in U.S. Pat. Nos. 7,557,189, 7,342,110, 8,119,787 and 8,337,855 and WO2004/043344, herein incorporated by reference. In another embodiment, the anti-CD33 antibody is huMy9-6 antibody.

In some embodiments, the anti-CD33 antibody comprises an immunoglobulin heavy chain region having the amino acid sequence of

(SEQ ID NO: 26)

QVQLQQPGAEVVKPGASVKMSCKASGYTFTSYYIHWIKQTPGQGLEWVGV
IYPGNDDISYNQKFQGKATLTADKSSTTAYMQLSSLTSEDSAVYYCAREV
RLRYFDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG,

and an immunoglobulin light chain region having the amino acid sequence of

(SEQ ID NO: 27)

EIVLTQSPGSLAVSPGERVTMSCKSSQSVFFSSSQKNYLAWYQQIPGQSP
RLLIYWASTRESGVPDRFTGSGSGTDFTLTISSVQPEDLAIYYCHQYLSS
RTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In yet another embodiment, the anti-CD33 antibody comprises the heavy chain CDR1-CDR3 of SEQ ID NO: 26, and/or the light chain CDR1-CDR3 of SEQ ID NO: 27, and preferably specifically binds CD33.

In yet another embodiment, the anti-CD33 antibody comprises a heavy chain variable region (HCVR) sequence at least about 90%, 95%, 97%, 99%, or 100% identical to SEQ ID NO: 26, and/or a light chain variable region (LCVR) sequence at least about 90%, 95%, 97%, 99%, or 100% identical to SEQ ID NO: 27, and preferably specifically binds CD33.

In another embodiment, the cell-binding agent is an anti-CD37 antibody or an antibody fragment thereof, such as those described in U.S. Pat. No. 8,765,917 and WO 2011/112978, herein incorporated by reference. In some embodiments, the anti-CD37 antibody is huCD37-3 antibody.

In some embodiments, the anti-CD37 antibody comprises an immunoglobulin light chain region having the amino acid sequence of

(SEQ ID NO: 28)

DIQMTQSPSSLSVSVGERVTITCRASENIRSNLAWYQQKPGKSPKLLVNV
ATNLADGVPSRFSGSGSGTDYSLKINSLQPEDFGTYYCQHYWGTTWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

and an immunoglobulin heavy chain region having the amino acid sequence of

(SEQ ID NO: 29)

QVQVQESGPGLVAPSQTLSITCTVSGFSLTTSGVSWVRQPPGKGLEWLGV
IWGDGSTNYHPSLKSRLSIKKDHSKSQVFLKLNSLTAADTATYYCAKGGY
SLAHWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG,

or an immunoglobulin heavy chain region having the amino acid sequence of

(SEQ ID NO: 30)

QVQVQESGPGLVAPSQTLSITCTVSGFSLTTSGVSWVRQPPGKGLEWLGV
IWGDGSTNYHSSLKSRLSIKKDHSKSQVFLKLNSLTAADTATYYCAKGGY
SLAHWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG

In yet another embodiment, the anti-CD37 antibody comprises an immunoglobulin light chain region having the amino acid sequence set forth in SEQ ID NO:28 and an immunoglobulin heavy chain region having the amino acid sequence set forth in SEQ ID NO:30.

In yet another embodiment, the anti-CD37 antibody comprises the heavy chain CDR1-CDR3 of SEQ ID NO: 29 or 30, and/or the light chain CDR1-CDR3 of SEQ ID NO: 28, and preferably specifically binds CD37.

In yet another embodiment, the anti-CD37 antibody comprises a heavy chain variable region (HCVR) sequence at least about 90%, 95%, 97%, 99%, or 100% identical to SEQ ID NO: 29 or 30, and/or a light chain variable region (LCVR) sequence at least about 90%, 95%, 97%, 99%, or 100% identical to SEQ ID NO: 28, and preferably specifically binds CD37.

In yet another embodiment, the anti-CD37 antibody comprises an immunoglobulin light chain region having the amino acid sequence of

(SEQ ID NO: 31)

EIVLTQSPATMSASPGERVTMTCSATSSVTYMHWYQQKPGQSPKRWIYDT
SNLPYGVPARFSGSGSGTSYSLTISSMEAEDAATYYCQQWSDNPPTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

and an immunoglobulin heavy chain region having the amino acid sequence of

(SEQ ID NO: 32)

QVQLQESGPGLLKPSQSLSLTCTVSGYSITSGFAWHWIRQHPGNKLEWMG
YILYSGSTVYSPSLKSRISITRDTSKNHFFLQLNSVTAADTATYYCARGY
YGYGAWFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG.

In yet another embodiment, the anti-CD37 antibody comprises the heavy chain CDR1-CDR3 of SEQ ID NO: 32, and/or the light chain CDR1-CDR3 of SEQ ID NO: 31, and preferably specifically binds CD37.

In yet another embodiment, the anti-CD37 antibody comprises a heavy chain variable region (HCVR) sequence at least about 90%, 95%, 97%, 99%, or 100% identical to SEQ ID NO: 32, and/or a light chain variable region (LCVR) sequence at least about 90%, 95%, 97%, 99%, or 100% identical to SEQ ID NO: 31, and preferably specifically binds CD37.

In yet another embodiment, the anti-CD37 antibody is huCD37-50 antibody.

In certain embodiments, the cell-binding agent of the present invention (e.g., antibody) have a N-terminal serine, which can be oxidized with an oxidizing agent to form an oxidized cell-binding agent having a N-terminal aldehyde group.

Any suitable oxidizing agent can be used in step (a) of the methods described above. In certain embodiments, the oxidizing agent is a periodate. More specifically, the oxidizing agent is sodium periodate.

Excess molar equivalents of the oxidizing agent relative to the cell-binding agent can be used. In certain embodiments, about 2-100, 5-80, 10-50, 1-10 or 5-10 molar equivalents of the oxidizing agent can be used. In certain embodiments, about 10 or about 50 equivalents of the oxidizing agent can be used. When large amount of the oxidizing agent is used, short reaction time is used to avoid over-oxidation. For example, when 50 equivalents of the oxidizing agent is used, the oxidation reaction is carried out for about 5 to about 60 minutes. Alternatively, when 10 equivalents of the oxidizing agent is used, the reaction is carried out for about 30 minutes to about 24 hours. In some embodiments, 5-10 molar equivalents of the oxidizing agent is used and the oxidation reaction is carried out for about 5 to about 60 minutes (e.g., about 10 to about 30 minutes, about 20 to about 30 minutes).

In certain embodiments, the oxidation reaction does not lead to significant non-targeted oxidation. For example, no signification extent (e.g., less than 20%, less than 10%, less than 5%, less than 3%, less than 2% or less than 1%) of methionine and/or glycans are oxidized during the oxidation process of N-terminal serine to generate the oxidized cell-binding agent having a N-terminal aldehyde group.

In certain embodiments, the cell-binding agent of the present invention (e.g., antibody) have a recombinantly engineered Cys residue, such as a Cys residue at EU/OU numbering position 442 of the antibody. Thus the term “cysteine engineered antibody” includes an antibody with at least one Cys that is not normally present at a given residue of the antibody light chain or heavy chain. Such Cys, which may also be referred to as “engineered Cys,” can be engineered using any conventional molecular biology or recombinant DNA technology (e.g., by replacing the coding sequence for a non-Cys residue at the target residue with a coding sequence for Cys). For example, if the original residue is Ser with a coding sequence of 5′-UCU-3′, the coding sequence can be mutated (e.g., by site-directed mutagenesis) to 5′-UGU-3′, which encodes Cys. In certain embodiments, the Cys engineered antibody of the invention has an engineered Cys in the heavy chain. In certain embodiments, the engineered Cys is in or near the CH3 domain of the heavy chain. The engineered antibody heavy (or light) chain sequence can be inserted into a suitable recombinant expression vector to produce the engineered antibody having the engineered Cys residue in place of the original Ser residue.

Production of Cell-Binding Agent-Drug Conjugates

In order to link the cytotoxic compounds or derivative thereof of the present invention to the cell-binding agent, the cytotoxic compound can comprise a linking moiety with a reactive group bonded thereto. These compounds can be directly linked to the cell-binding agent. Representative processes for linking the cytotoxic compounds having a reactive group bonded thereof with the cell-binding agent to produce the cell-binding agent-cytotoxic agent conjugates are described in Examples 3 and 4.

In some embodiments, a bifunctional crosslinking reagent can be first reacted with the cytotoxic compound to provide the compound bearing a linking moiety with one reactive group bonded thereto (i.e., drug-linker compound), which can then react with a cell binding agent. Alternatively, one end of the bifunctional crosslinking reagent can first react with the cell binding agent to provide the cell binding agent bearing a linking moiety with one reactive group bonded thereto, which can then react with a cytotoxic compound. The linking moiety can contain a chemical bond that allows for the release of the cytotoxic moiety at a particular site. Suitable chemical bonds are well known in the art and include disulfide bonds, thioether bonds, acid labile bonds, photolabile bonds, peptidase labile bonds and esterase labile bonds (see for example U.S. Pat. Nos. 5,208,020; 5,475,092; 6,441,163; 6,716,821; 6,913,748; 7,276,497; 7,276,499; 7,368,565; 7,388,026 and 7,414,073). Preferred are disulfide bonds, thioether and peptidase labile bonds. Other linkers that can be used in the present invention include non-cleavable linkers, such as those described in are described in detail in U.S. publication number 2005/0169933, or charged linkers or hydrophilic linkers and are described in US 2009/0274713, US 2010/01293140 and WO 2009/134976, each of which is expressly incorporated herein by reference, each of which is expressly incorporated herein by reference.

In some embodiments, a solution of a cell-binding agent (e.g., an antibody) in aqueous buffer may be incubated with a molar excess of a bifunctional crosslinking agent, such as N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. The modified cell-binding agent (e.g., modified antibody) is then reacted with the thiol-containing cytotoxic compound described herein, such as compound 11 (Example 2), to produce a disulfide-linked cell-binding agent-cytotoxic agent conjugate of the present invention.

In another embodiment, the thiol-containing cytotoxic compound described herein, such as compound 11 can react with a bifunctional crosslinking agent such as N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to form a cytotoxic agent-linker compound, which can then react with a cell-biding agent to produce a disulfide-linked cell-binding agent-cytotoxic agent conjugate of the present invention. The cytotoxic agent-linker compound can be prepared in situ without purification before reacting with the cell-binding agent. Alternatively, the cytotoxic agent-linker compound can be purified prior to reacting with the cell-binding agent.

The cell binding agent-cytotoxic agent conjugate may be purified using any purification methods known in the art, such as those described in U.S. Pat. No. 7,811,572 and US Publication No. 2006/0182750, both of which are incorporated herein by reference. For example, the cell-binding agent-cytotoxic agent conjugate can be purified using tangential flow filtration, adsorptive chromatography, adsorptive filtration, selective precipitation, non-absorptive filtration or combination thereof. Preferably, tangential flow filtration (TFF, also known as cross flow filtration, ultrafiltration and diafiltration) and/or adsorptive chromatography resins are used for the purification of the conjugates.

Alternatively, the cell-binding agent (e.g., an antibody) may be incubated with a molar excess of an antibody modifying agent such as 2-iminothiolane, L-homocysteine thiolactone (or derivatives), or N-succinimidyl-S-acetylthioacetate (SATA) to introduce sulfhydryl groups. The modified antibody is then reacted with the appropriate disulfide-containing cytotoxic agent, to produce a disulfide-linked antibody-cytotoxic agent conjugate. The antibody-cytotoxic agent conjugate may then be purified by methods described above. The cell binding agent may also be engineered to introduce thiol moieties, such as cysteine-engineered antibodies disclosed in U.S. Pat. Nos. 7,772,485 and 7,855,275.

In another embodiment, a solution of a cell-binding agent (e.g., an antibody) in aqueous buffer may be incubated with a molar excess of an antibody-modifying agent such as N-succinimidyl-4-(N-maleimidomethyl)-cyclohexane-1-carboxylate to introduce maleimido groups, or with N-succinimidyl-4-(iodoacetyl)-aminobenzoate (SIAB) to introduce iodoacetyl groups. The modified cell-binding agent (e.g., modified antibody) is then reacted with the thiol-containing cytotoxic agent to produce a thioether-linked cell-binding agent-cytotoxic agent conjugate. The conjugate may then be purified by methods described above.

The number of cytotoxic molecules bound per antibody molecule can be determined spectrophotometrically by measuring the ratio of the absorbance at 280 nm and 330 nm. In some embodiments, an average of 1-10 cytotoxic compounds/antibody molecule(s) can be linked by the methods described herein. In some embodiments, the average number of linked cytotoxic compounds per antibody molecule is 2-5, and more specifically 2.5-4.0.

Representative processes for preparing the cell-binding agent-drug conjugates of the present invention are described in U.S. Pat. No. 8,765,740 and U.S. Application Publication No. 2012/0238731. The entire teachings of these references are incorporated herein by reference.

Cytotoxicity of Compounds and Conjugates

The cytotoxic compounds and cell-binding agent-drug conjugates of the invention can be evaluated for their ability to suppress proliferation of various cancer cell lines in vitro. For example, cell lines such as human cervical carcinoma cell line KB, human acute monocytic leukemia cell line THP-1, human promyelocytic leukemia cell line HL60, human acute myeloid leukaemia cell line HNT-34, can be used for the assessment of cytotoxicity of these compounds and conjugates. Cells to be evaluated can be exposed to the compounds or conjugates for 1-5 days and the surviving fractions of cells measured in direct assays by known methods. IC₅₀ values can then be calculated from the results of the assays. Alternatively or in addition, an in vitro cell line sensitivity screen, such as the one described by the U.S. National Cancer Institute (see Voskoglou-Nomikos et al., 2003, Clinical Cancer Res. 9: 42227-4239, incorporated herein by reference) can be used as one of the guides to determine the types of cancers that may be sensitive to treatment with the compounds or conjugates of the invention.

Examples of in vitro potency and target specificity of antibody-cytotoxic agent conjugates of the present invention are described in Example 7. Antigen negative cell lines remained viable when exposed to the same conjugates.

Compositions and Methods of Use

The present invention includes a composition (e.g., a pharmaceutical composition) comprising novel benzodiazepine compounds described herein, derivatives thereof, or conjugates thereof, (and/or solvates, hydrates and/or salts thereof) and a carrier (a pharmaceutically acceptable carrier). The present invention also includes a composition (e.g., a pharmaceutical composition) comprising novel benzodiazepine compounds described herein, derivatives thereof, or conjugates thereof, (and/or solvates, hydrates and/or salts thereof) and a carrier (a pharmaceutically acceptable carrier), further comprising a second therapeutic agent. The present compositions are useful for inhibiting abnormal cell growth or treating a proliferative disorder in a mammal (e.g., human). The present compositions are also useful for treating depression, anxiety, stress, phobias, panic, dysphoria, psychiatric disorders, pain, and inflammatory diseases in a mammal (e.g., human).

The present invention includes a method of inhibiting abnormal cell growth or treating a proliferative disorder in a mammal (e.g., human) comprising administering to said mammal a therapeutically effective amount of novel benzodiazepine compounds described herein, derivatives thereof, or conjugates thereof, (and/or solvates and salts thereof) or a composition thereof, alone or in combination with a second therapeutic agent.

The present invention also provides methods of treatment comprising administering to a subject in need of treatment an effective amount of any of the conjugates described above.

Similarly, the present invention provides a method for inducing cell death in selected cell populations comprising contacting target cells or tissue containing target cells with an effective amount of a cytotoxic agent comprising any of the cytotoxic compound-cell-binding agents of the present invention, a salt or solvate thereof. The target cells are cells to which the cell-binding agent can bind.

If desired, other active agents, such as other anti-tumor agents, may be administered along with the conjugate.

Suitable pharmaceutically acceptable carriers, diluents, and excipients are well known and can be determined by those of ordinary skill in the art as the clinical situation warrants.

Examples of suitable carriers, diluents and/or excipients include: (1) Dulbecco's phosphate buffered saline, pH about 7.4, containing or not containing about 1 mg/mL to 25 mg/mL human serum albumin, (2) 0.9% saline (0.9% w/v NaCl), and (3) 5% (w/v) dextrose; and may also contain an antioxidant such as tryptamine and a stabilizing agent such as Tween 20.

The method for inducing cell death in selected cell populations can be practiced in vitro, in vivo, or ex vivo.

Examples of in vitro uses include treatments of autologous bone marrow prior to their transplant into the same patient in order to kill diseased or malignant cells: treatments of bone marrow prior to their transplantation in order to kill competent T cells and prevent graft-versus-host-disease (GVHD); treatments of cell cultures in order to kill all cells except for desired variants that do not express the target antigen; or to kill variants that express undesired antigen.

The conditions of non-clinical in vitro use are readily determined by one of ordinary skill in the art.

Examples of clinical ex vivo use are to remove tumor cells or lymphoid cells from bone marrow prior to autologous transplantation in cancer treatment or in treatment of autoimmune disease, or to remove T cells and other lymphoid cells from autologous or allogenic bone marrow or tissue prior to transplant in order to prevent GVHD.

Treatment can be carried out as follows. Bone marrow is harvested from the patient or other individual and then incubated in medium containing serum to which is added the cytotoxic agent of the invention, concentrations range from about 10 μM to 1 pM, for about 30 minutes to about 48 hours at about 37° C. The exact conditions of concentration and time of incubation, i.e., the dose, are readily determined by one of ordinary skill in the art. After incubation the bone marrow cells are washed with medium containing serum and returned to the patient intravenously according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

For clinical in vivo use, the cytotoxic agent of the invention will be supplied as a solution or a lyophilized powder that are tested for sterility and for endotoxin levels. Examples of suitable protocols of conjugate administration are as follows. Conjugates are given weekly for 4 weeks as an intravenous bolus each week. Bolus doses are given in 50 to 1000 mL of normal saline to which 5 to 10 mL of human serum albumin can be added. Dosages will be 10 μg to 2000 mg per administration, intravenously (range of 100 ng to 20 mg/kg per day). After four weeks of treatment, the patient can continue to receive treatment on a weekly basis. Specific clinical protocols with regard to route of administration, excipients, diluents, dosages, times, etc., can be determined by one of ordinary skill in the art as the clinical situation warrants.

Examples of medical conditions that can be treated according to the in vivo or ex vivo methods of inducing cell death in selected cell populations include malignancy of any type including, for example, cancer, autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, AIDS, etc.; and parasite infections, such as giardiasis, amoebiasis, schistosomiasis, and others as determined by one of ordinary skill in the art.

In some embodiments, the compounds and conjugates of the present invention can be used for treating cancer (e.g., ovarian cancer, pancreatic cancer, cervical cancer, melanoma, lung cancer (e.g., non small-cell lung cancer and small-cell lung cancer), colorectal cancer, breast cancer (e.g., triple negative breast cancer (TNBC)), gastric cancer, squamous cell carcinoma of the head and neck, prostate cancer, endometrial cancer, sarcoma, multiple myeloma, head and neck cancer, blastic plasmacytoid dendritic neoplasm (BPDN), lymphoma (e.g., non-Hodgkin lymphoma), myelodysplastic syndrome (MDS), peritoneal cancer, or leukemia (e.g., acute myeloid leukemia (AML), acute monocytic leukemia, promyelocytic leukemia, eosinophilic leukaemia, acute lymphoblastic leukemia (e.g., B-ALL), chronic lymphocytic leukemia (CLL), and chronic myeloid leukemia (CML))

Cancer therapies and their dosages, routes of administration and recommended usage are known in the art and have been described in such literature as the Physician's Desk Reference (PDR). The PDR discloses dosages of the agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician. The contents of the PDR are expressly incorporated herein in its entirety by reference. One of skill in the art can review the PDR, using one or more of the following parameters, to determine dosing regimen and dosages of the chemotherapeutic agents and conjugates that can be used in accordance with the teachings of this invention. These parameters include:

Comprehensive index

### By Manufacturer

Products (by company's or trademarked drug name)
Category index
Generic/chemical index (non-trademark common drug names)
Color images of medications
Product information, consistent with FDA labeling
Chemical information

### Function/action

### Indications & Contraindications

Trial research, side effects, warnings

Synthetic Precursors for the Compounds of the Present Invention and Methods of Making Thereof

In a third aspect, the present invention provides a monomer compound represented by the following formula:

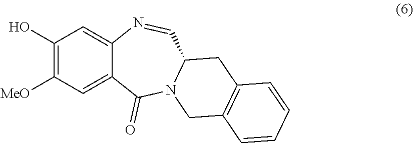 [SMILES: COc1cc2c(cc1O)N=C[C@@H]1Cc3ccccc3CN1C2=O]

or a salt thereof. The monomer compound can be used in preparing the cytotoxic compound of formula (I) of the present invention or a pharmaceutically acceptable salt thereof.

In certain embodiments, the compound of formula (6) can be prepared according to the following scheme:

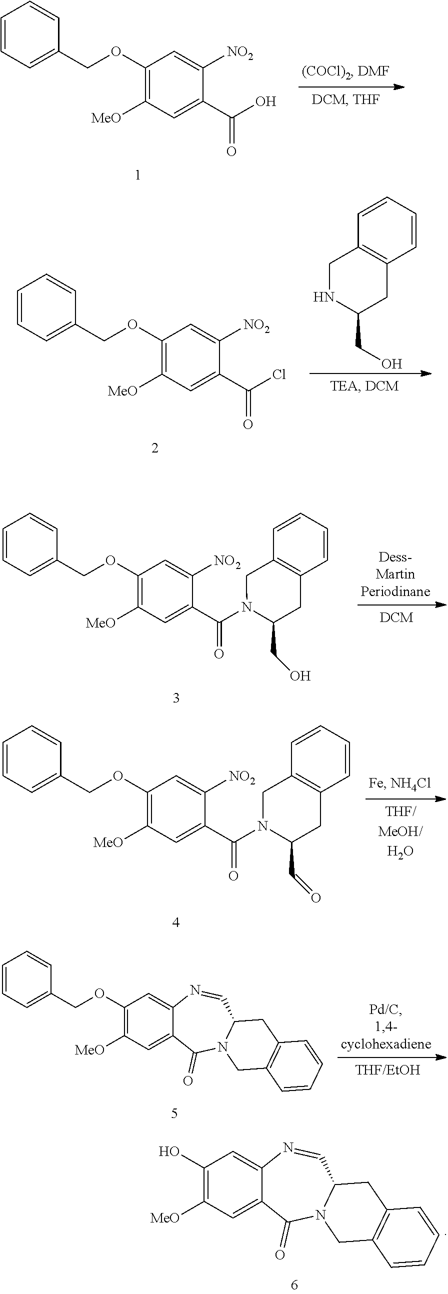

In a first embodiment of the third aspect, the compound of formula (6) can be prepared comprising the steps of:

a) reacting the compound of formula (4):

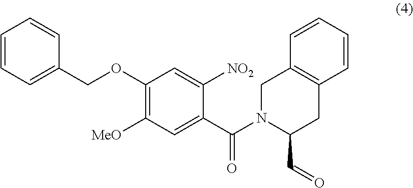 [SMILES: COc1cc(C(=O)N2Cc3ccccc3C[C@H]2C=O)c([N+](=O)[O-])cc1OCc1ccccc1]

with Fe in the presence of NH₄Cl to form a compound of formula (5):

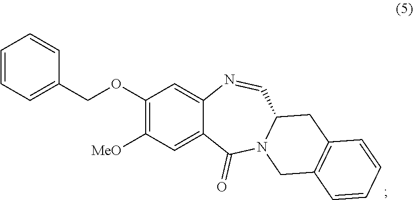 [SMILES: COc1cc2c(cc1OCc1ccccc1)N=C[C@@H]1Cc3ccccc3CN1C2=O]

and

b) reacting the compound of formula (5) with a hydrogenation reagent in the presence of a palladium catalyst to form the compound of formula (6).

In a second embodiment of the third aspect, the present invention provides a method of preparing a compound of formula (5) comprising reacting the compound of formula (4):

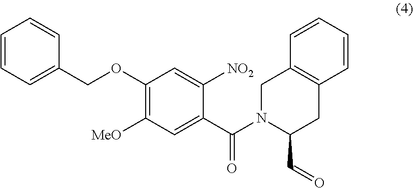 [SMILES: COc1cc(C(=O)N2Cc3ccccc3C[C@H]2C=O)c([N+](=O)[O-])cc1OCc1ccccc1]

with Fe in the presence of NH₄Cl to form a compound of formula (5).

In a third embodiment of the third aspect, the present invention provides a method of preparing a compound of formula (6) comprising reacting the compound of formula (5) with a hydrogenation reagent in the presence of a palladium catalyst to form the compound of formula (6).

In a 1^{st} specific embodiment, for the method of the first or second embodiment of the third aspect, the reaction of the compound of formula (4) and Fe/NH₄Cl is carried out in a solvent or a solvent mixture. Any suitable solvent or solvent mixtures can be used. Exemplary solvents include, but are not limited to, tetrahydrofuran (THF), 2-methyltetrahydrofuran (MeTHF), N-methyl-2-pyrrolidone (NMP), methanol, ethanol, isopropanol, dichloromethane, dichloroethane, acetonitrile, dimethylformamide (DMF), dimethylacetamide, cyclopentyl methyl ether (CPME), ethyl acetate, water, and a combination thereof. In certain embodiment, the reaction is carried out in a mixture of water and one or more organic solvents. Any suitable organic solvents described above can be used. In a more specific embodiment, the reaction is carried out in a mixture of THF, methanol and water.

In a 2^{nd} specific embodiment, for the method of the first or second embodiment or the 1^{st} specific embodiment of the third aspect, the reaction between the compound of formula (4) and Fe/NH₄Cl is carried out at a temperature between 0° C. and 100° C., between 20° C. and 100° C., between 40° C. and 90° C., between 50° C. and 80° C., or between 40° C. and 60° C. In a more specific embodiment, the reaction is carried out at 50° C.

As used herein, the term “between number 1 and number 2” means a number that is greater or equal to number 1 and less or equal to number 2.

As used herein, the term “number 1 to number 2” means a number that is greater or equal to number 1 and less or equal to number 2.

In certain embodiments, for the method of the first or second embodiment or the 1^{st} or 2^{nd} specific embodiment of the third aspect, the reaction between the compound of formula (4) and Fe/NH₄Cl can be carried out for appropriate amount of time, such as 1 hour to 1 week, 4 hours to 72 hours, 10 hours to 72 hours, 24 hours to 72 hours, 4 hours to 10 hours, or 10 hours to 24 hours. In a specific embodiment, the reaction is carried out for 12 hours.

In certain embodiments, for the method of the first or second embodiment or the 1^{st} or 2^{nd} specific embodiment of the third aspect, the reaction between the compound of formula (4) and Fe/NH₄Cl is carried out under an inert atmosphere, such as under N₂, Ar etc. In a specific embodiment, the reaction is carried out under N₂ atmosphere.

In certain embodiments, for the method of the first or second embodiment or the 1^{st} or 2^{nd} specific embodiment of the third aspect, the compound of formula (5) obtained from the reaction between the compound of formula (4) and Fe/NH₄Cl is purified. Any suitable purification methods, such as precipitation, re-crystallization, column chromatography or a combination thereof, can be used. In certain embodiments, precipitation, re-crystallization, or a combination thereof can be used to purify the compound of formula (5). Multiple (e.g., two, three, four, etc.) precipitations or re-crystallizations or a combination therefore can be used to purify the compound of formula (4).

As used herein, “re-crystallization” refers to a process for purifying a solid material, wherein the atoms, molecules or ions of the purified solid material obtained are arranged in highly organized structure(s), known as crystalline form(s). Re-crystallization can be achieved by various methods, such as cooling, evaporation, addition of a second solvent (i.e., antisolvent), etc.

As used herein, “precipitation” refers to a purification process in which solid material forms from a solution having the solid material dissolved therein. Precipitation can often achieved by cooling down the temperature of the solution or adding a second solvent (i.e., antisolvent) that significantly reduce the solubility of the desired solid material in the solution. The solid material obtained from the precipitation process can be in one or more amorphous forms, one or more crystalline forms or a combination thereof.

In a 3^{rd} specific embodiment of the third aspect, for the method of the first or second embodiment or the 1^{st} or 2^{nd} specific embodiment, the compound of formula (5) obtained from the reaction between the compound of formula (4) and Fe/NH₄Cl is purified by re-crystallization or precipitation in a mixture of dichloromethane and ethanol. In a more specific embodiment, the volume ratio of dichloromethane and ethanol is between 5:1 and 1:2, between 4:1 and 1:1.5, between 3:1 and 1:1.5, or between 2:1 and 1:1.2. In a specific embodiment, the volume ratio of dichoromethane and ethanol is 1:1. In certain embodiments, the re-crystallization is carried out overnight.

Alternatively, the compound of formula (5) is purified by re-crystallization or precipitation in a mixture of toluene and acetonitrile. In one embodiment, the compound of formula (I) or (IA) is dissolved in toluene at an elevated temperature, such as a temperature between 40° C. and 90° C., between 50° C. and 90° C., between 60° C. and 90° C., between 70° C. and 90° C., or between 75° C. and 85° C. In another even more specific embodiment, the compound of formula (5) is dissolved in toluene at 80° C. followed by addition of acetonitrile, to re-crystalize or precipitate the compound of formula (5). Optionally, the compound of formula (5) is filtered after dissolution in toluene before the addition of acetonitrile. In one embodiment, the volume ratio of toluene and acetonitrile is between 1:10 and 2:1, between 1:5 and 1:1, between 1:3 and 1:1, or between 1:2 and 1:1. In a specific embodiment, the volume ratio of toluene and acetonitrile is 1:1.5.

In a 4^{th} specific embodiment, for the methods of the 3^{rd} specific embodiment of the third aspect described above, the compound of formula (5) is further purified by recrystallization or precipitation. In a more specific embodiment, the compound of formula (5) is further purified by recrystallization or precipitation in a mixture of toluene and acetonitrile. In a even more specific embodiment, the compound of formula (5) is dissolved in toluene at an elevated temperature, such as a temperature between 40° C. and 90° C., between 50° C. and 90° C., between 60° C. and 90° C., between 70° C. and 90° C., or between 75° C. and 85° C. In another even more specific embodiment, the compound of formula (5) is dissolved in toluene at 80° C. followed by addition of acetonitrile, to re-crystalize or precipitate the compound of formula (5). Optionally, the compound of formula (5) is filtered after dissolution in toluene before the addition of acetonitrile. In one embodiment, the volume ratio of toluene and acetonitrile is between 1:10 and 2:1, between 1:5 and 1:1, between 1:3 and 1:1, or between 1:2 and 1:1. In a specific embodiment, the volume ratio of toluene and acetonitrile is 1:1.5.

In a 5^{th} specific embodiment of the third aspect, for the method of the first or third embodiment or the 1^{st}, 2^{nd}, 3^{rd} or 4^{th} specific embodiment of the third aspect, the de-benzylation reaction of the compound of formula (5) is carried out in the presence of a Pd/Alox (also known as palladium on alumina (i.e., aluminum oxide)) catalyst. Any suitable Pd/Alox catalysts can be used. Exemplary palladium/Alox catalysts include, but are not limited to, palladium on alumina 10% Pd basis (i.e., 10 w.t. % Pd/Alox), such as Sigma-Aldrich® #76000, palladium on alumina 5% Pd basis (i.e., 5 w.t. % Pd/Alox), such as Johnson Matthey 5R325 Powder, Johnson Matthey A302099-5, Noblyst® P1159, STREM 46-1960, 46-1951, palladium on alumina 0.5% Pd basis (i.e., 0.5 w.t. % Pd/Alox), such as STREM 46-1920, Alfa Aesar #41383, #38786, #89114, #38289. In a more specific embodiment, the palladium catalyst is 5 w.t. % Pd/Alox (i.e., palladium on alumina 5% Pd basis).

In a 6^{th} specific embodiment, for the method of the first or third embodiment or the 1^{st}, 2^{nd}, 3^{rd} or 4^{th} specific embodiment of the third aspect, the de-benzylation reaction of the compound of formula (5) is carried out in the presence of Pd/C (also known as palladium on carbon). Any suitable Pd/C catalysts can be used. Exemplary Pd/C catalysts include, but are not limited to, palladium on activated carbon 20% Pd basis (i.e., 20 w.t. % Pd/C), such as STREM 46-1707, palladium on activated charcoal 10% Pd basis (i.e., 10 w.t. % Pd/C), such as Sigma-Aldrich® #75990, #75993, Johnson Matthey 10R39, 10R394, 10R487 Powder, 10R87L Powder, 10T755, Evonik Noblyst® P1070, STREM 46-1900, palladium on activated charcoal 5% Pd basis (i.e., 5 w.t. % Pd/C), such as Sigma-Aldrich® #75992, #75991, Johnson Matthey 5R338M, 5R369, 5R374, 5R39, 5R395, 5R424, 5R434, 5R437, 5R440, 5R452, 5R487, 5R487 Powder, 5R58, 5R87L, 5T761, A102023-5, A103023-5, A105023-5, A302002-5, A302023-10, A302023-5, A402028-10, A405028-5, A405032-5, A405129-5, A501023-10, A503002-5, A503023-5, A503032-5, A702023-10, STREM 46-1890, 46-1908, 46-1909, 46-1911, Eonik Noblyst® P1086, P1090, P1092, P1109, palladium on activated carbon 3% Pd basis (i.e., 3 w.t. % Pd/C), such as STREM 46-1907, palladium on activated carbon 0.5% Pd basis (i.e., 0.5 w.t. % Pd/Alox), such as Alfa Aesar #38289.

In a 7^{th} specific embodiment, for the method of the 5^{th} or 6^{th} specific embodiment of the third aspect, the de-benzylation reaction of the compound of formula (5) is carried out in the presence of 0.05 to 0.5 equivalent of Pd for every 1 equivalent of the compound of formula (5)). In one embodiment, between 0.05 and 0.4, between 0.05 and 0.35, between 0.05 and 0.3, between 0.05 and 0.25, between 0.05 and 0.2, between 0.05 and 0.15, between 0.075 and 0.15, between 0.075 and 0.1, between 0.08 and 0.1 or between 0.1 to 0.3 equivalent of Pd catalyst is used for every 1 equivalent of the compound of formula (5). In a more specific embodiment, 0.15 to 0.25 equivalent of the Pd catalyst is used for every 1 equivalent of the compound of formula (5). In another embodiment, the amount of the palladium catalyst used depends on the type and manufacturer of the palladium catalyst used and the suitable amount of the palladium catalyst can be determined experimentally.

In a 8^{th} specific embodiment, for the method of the first or third embodiment or the 1^{st}, 2^{nd}, 3^{rd}, 4^{th}, 5^{th}, 6^{th} or 7^{th} specific embodiment of the third embodiment, the de-benzylation reaction of the compound of formula (5) is carried out in the presence of 1,4-cyclohexadiene and a palladium catalyst (e.g., those described in the 5^{th} or 6^{th} specific embodiment). In one embodiment, 1.0 to 10.0 equivalents of 1,4-cyclohexadiene is used for every 1 equivalent of the compound of formula (5). In another embodiment, 1.0 to 4.5, 1.0 to 4.0, 1.0 to 3.5, 1.0 to 3.0, 1.0 to 2.5, 1.1 to 2.0, 1.3 to 1.8, 1.5 to 1.7, 6.0 to 10.0, 7.0 to 9.0, or 7.5 to 8.5 equivalents of 1,4-cyclohexadiene is used for every 1 equivalent of the compound of formula (5).

In a 9^{th} specific embodiment, for the method of the first or third embodiment or the 1^{st}, 2^{nd}, 3^{rd}, 4^{th}, 5^{th}, or 6^{th} specific embodiment of the third aspect, the de-benzylation reaction comprises reacting the compound of formula (5) with 1,4-cyclohexadiene in the presence of a Pd/C catalyst (e.g., 10% Pd/C), and wherein 6.0 to 8.0 equivalent of 1,4-cyclohexadiene and 0.1 to 0.7 equivalent of Pd are used for every 1 equivalent of the compound of formula (5). In a more specific embodiment, 7.0 to 9.0 equivalent of 1,4-cyclohexadiene and 0.15 to 0.25 equivalent of a Pd/C catalyst (e.g., 10% Pd/C) are used for every 1 equivalent of the compound of formula (5).

In a 10^{th} specific embodiment, for the method of the first or third embodiment or the 1^{st}, 2^{nd}, 3^{rd}, 4^{th}, 5^{th}, 6^{th}, 7^{th}, 8^{th} or 9^{th} specific embodiment, the de-benzylation reaction is carried out in a solvent or a mixture of solvents. Any suitable solvents described herein can be used. Exemplary solvents include, but are not limited to, tetrahydrofuran (THF), 2-methyltetrahydrofuran (MeTHF), N-methyl-2-pyrrolidone (NMP), methanol, ethanol, isopropanol, dichloromethane, dichloroethane, acetonitrile, dimethylformamide (DMF), dimethylacetamide, cyclopentyl methyl ether (CPME), ethyl acetate, water, and a combination thereof. In a more specific embodiment, the de-benzylation reaction is carried out in a solvent mixture comprising a Pd-catalyst poison such as lead, copper, sulfur, sulfur-containing compounds, nitrogen-containing heterocycles or amines. In some embodiments, the Pd-catalyst poison is a thiol, thophene, pyridine, quinoline, 3,6-dithia-1,8-octanediol or DMSO. In an even more specific embodiment, the de-benzylation reaction is carried out in a mixture of DMSO and ethanol. DMSO can be present in a very small amount. For example, the solvent mixture (e.g., DMSO and ethanol) can have 0.01-1%, 0.05-0.75%, 0.1-0.5%, 0.1-0.3% or 0.1-0.2% by volume of DMSO. In another more specific embodiment, the de-benzylation reaction is carried out in a mixture of THF and ethanol.

In a 11^{th} specific embodiment, for the method of the first or third embodiment or the 1^{st}, 2^{nd}, 3^{rd}, 4^{th}, 5^{th,} 6^{th}, 7^{th}, 8^{th}, 9^{th} or 10^{th} specific embodiment of the third aspect, the de-benzylation reaction is carried out at a temperature between 10° C. and 90° C., between 15° C. to 30° C., between 40° C. and 70° C., between 40° C. and 60° C., or between 45° C. and 55° C. In a more specific embodiment, the reaction is carried out at 50° C. In another more specific embodiment, the reaction is carried out at room temperature.

In a 12^{th} specific embodiment, for the method of the first or second embodiment, or the 1^{st}, 2^{nd}, 3^{rd}, 4^{th}, 5^{th}, 6^{th}, 7^{th}, 8^{th}, 9^{th}, 10^{th} or 11^{th} specific embodiment of the third aspect, the compound of formula (4) is prepared by a method comprising oxidizing the compound of formula (3):

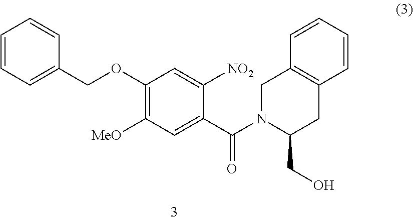 [SMILES: COc1cc(C(=O)N2Cc3ccccc3C[C@H]2CO)c([N+](=O)[O-])cc1OCc1ccccc1]

with an oxidizing agent to form the compound of formula (4). In certain embodiments, the oxidizing agent is Dess-Martin periodinane (DMP), 2-iodoxybenzoic acid, Collins reagent (CrO₃.Py₂), pyridinium dichromate (PDC), pyridinium chlorochromate (PCC), tetrapropylammonium perruthenate (TPAP)/N-methylmorpholine N-oxide (NMO), (2,2,6,6-tetramethylpiperidin-1-yl)oxyl (TEMPO)/NaClO, DMSO/oxalyl chloride, DMSO/carbodiimide or DMSO/SO₃. Py. In a more specific embodiment, the oxidizing agent is DMP.

In certain embodiments, excess amount of the oxidizing agent relative to the compound of formula (3) can be used. For example, 1.01 to 10 equivalent, 1.01 to 5 equivalent, 1.05 to 2.0 equivalent, or 1.1 to 1.5 equivalent of the oxidizing agent can be used for every 1 equivalent of the compound of formula (3).

The oxidation reaction can be carried out in a suitable solvent or solvent mixtures described herein. In one embodiment, the reaction is carried out in dichloromethane.

The oxidation reaction can be carried out at a suitable temperature, for example, at a temperature between 0° C. to 50° C., between 0° C. to 30° C., or between 10° C. to 25° C. In one embodiment, the oxidation reaction is carried out at room temperature or 20° C.

In a 13^{th} specific embodiment, for the method of the 12^{th} specific embodiment of the third aspect, the compound of formula (3) is prepared by a method comprising reacting a compound of formula (2):

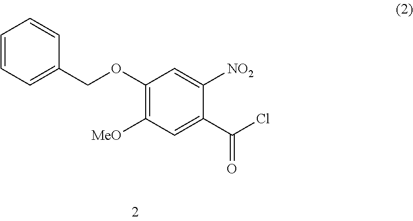 [SMILES: COc1cc(C(=O)Cl)c([N+](=O)[O-])cc1OCc1ccccc1]

with a compound of formula (a):

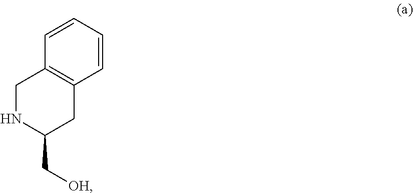 [SMILES: OC[C@@H]1Cc2ccccc2CN1]

to form the compound of formula (3).

In a 14^{th} specific embodiment, for the method of the 12^{th} specific embodiment, the compound of formula (3) is prepared by a method comprising reducing the compound of formula (3a):

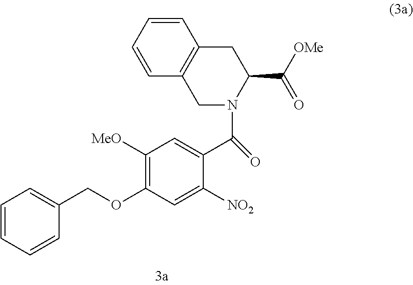 [SMILES: COC(=O)[C@@H]1Cc2ccccc2CN1C(=O)c1cc(OC)c(OCc2ccccc2)cc1[N+](=O)[O-]]

with a reducing agent to form the compound of formula (3). In certain embodiments, the reducing agent is a hydride reducing agent. In certain embodiments, the reducing agent is sodium borohydride, sodium triacetoxy borohydride, sodium cyanoborohydride, lithium aluminum hydride, hydrogen gas, ammonium formate, borane, 9-borabicyclo[3.3.1]nonane (9-BBN), diisobutylaluminium hydride (DIBAL), lithium borohydride (LiBH₄), potassium borohydride (KBH₄), or sodium bis(2-methoxyethoxy)aluminumhydride (Red-Al). In a more specific embodiment, the reducing agent is sodium borohydride.

In certain embodiment, excess amount of the reducing agent relative to the compound of formula (3a) can be used. For example, 1.1 to 10 equivalents, 1.5 to 5 equivalents, 2.0 to 4.0 equivalents, or 2.5 to 3.5 equivalents of the reducing agent can be used for every 1 equivalent of the compound of formula (3a).

In certain embodiment, the reduction reaction can be carried out in a suitable solvent or solvent mixtures described herein. In one embodiment, the reaction is carried out in the mixture of THF and ethanol.

The reduction reaction can be carried out at a suitable temperature, for example, at a temperature between 0° C. to 50° C., between 0° C. to 30° C., or between 10° C. to 25° C. In one embodiment, the reduction reaction is carried out at room temperature or 20° C.

Analogues and Derivatives

One skilled in the art of cytotoxic agents will readily understand that each of the cytotoxic agents described herein can be modified in such a manner that the resulting compound still retains the specificity and/or activity of the starting compound. The skilled artisan will also understand that many of these compounds can be used in place of the cytotoxic agents described herein. Thus, the cytotoxic agents of the present invention include analogues and derivatives of the compounds described herein.

All references cited herein and in the examples that follow are expressly incorporated by reference in their entireties.

### EXAMPLES

The invention will now be illustrated by reference to non-limiting examples. Unless otherwise stated, all percentages, ratios, parts, etc. are by weight. All reagents were purchased from the Aldrich Chemical Co., New Jersey, or other commercial sources. Nuclear Magnetic Resonance (¹H NMR) spectra were acquired on a Bruker 400 MHz instrument. Mass spectra were acquired on a Bruker Daltonics Esquire 3000 instrument and LCMS were acquired on an Agilent 1260 Infinity LC with an Agilent 6120 single quadropole MS using electrospray ionization.

The following solvents, reagents, protecting groups, moieties and other designations may be referred to by their abbreviations in parenthesis:

Me=methyl; Et=ethyl; Pr=propyl; i-Pr=isopropyl; Bu=butyl; t-Bu=tert-butyl; Ph=phenyl, and Ac=acetyl

AcOH or HOAc=acetic acid
ACN or CH₃CN=acetonitrile
Ala=alanine
aq=aqueous
Ar=argon
Bn=benzyl
Boc or BOC=tert-butoxycarbonyl
CBr₄=carbontetrabromide
Cbz or Z=benzyloxycarbonyl
DCM or CH₂Cl₂=dichloromethane
DCE=1,2-dichloroethane
DMAP=4-dimethylaminopyridine
DI water=deionized water

### DIEA or DIPEA=N,N-diisopropylethylamine

### DMA=N,N-dimethylacetamide

### DMF=N,N-dimethylformamide

### DMP=Dess-Martin Periodinane

DMSO=dimethyl sulfoxide
EDC=1-ethyl-3-(3-dimethylaminopropyl)carbodiimide
EEDQ=N-Ethoxycarbonyl-2-ethoxy-1,2-dihydroquinoline
ESI or ES=electrospray ionization
EtOAc=ethylacetate
g=grams
h=hour
HPLC=high-performance liquid chromatography
HOBt or HOBT=1-hydroxybenzotriazole
LC=liquid chromatography
LCMS=liquid chromatography mass spectrometry
min=minutes
mg=miligrams
mL=mililiters
mmol=milimoles
μg=micrograms
μL=microliters
μmol=micromoles
Me=methyl
MeOH=methanol
MS=mass spectrometry
MsCl=methanesulfonyl chloride (mesyl chloride)
Ms₂O=methanesulfonic anhydride
NaBH(OAc)₃=sodium triacetoxyborohydride

### NHS=N-hydroxysuccinamide

NMR=nuclear magnetic resonance spectroscopy
PPh₃=triphenylphosphine
RPHPLC or RP-HPLC=reverse phase high-performance liquid chromarography
RT or rt=room temperature (ambient, about 25° C.)
sat or sat'd=saturated
STAB=sodium triacetoxyborohydride (NaBH(OAc)₃)
TBSCl or TBDMSCl=tert-butyldimethylsilyl chloride
TBS=tert-butyldimethylsilyl
TCEP. HCl=tris(2-carboxyethyl)phosphine hydrochloride salt
TEA=triethylamine (Et₃N)
TFA=trifluoroacetic acid
THF=tetrahydrofuran

### Example 1. Synthesis of THIQ-Benzodiazepine Monomer, 6

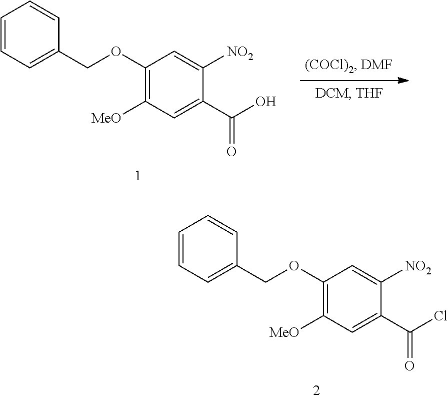 [SMILES: COc1cc(C(=O)O)c([N+](=O)[O-])cc1OCc1ccccc1 ; COc1cc(C(=O)Cl)c([N+](=O)[O-])cc1OCc1ccccc1]

Step 1:

Oxalyl chloride (3.61 mL, 41.2 mmol) was added dropwise to a stirred solution of compound 1 (5.0 g, 16.49 mmol) in DCM (42.8 mL), THF (4.28 mL) and DMF (0.020 mL, 0.264 mmol) at 0° C. under Ar. The reaction mixture was warmed to rt and was stirred for 3 h. The reaction mixture was concentrated and placed under high vacuum to obtain compound 2 as a pale yellow solid and was taken onto the next step without purification (5.3 g, 16.49 mmol, 100% yield).

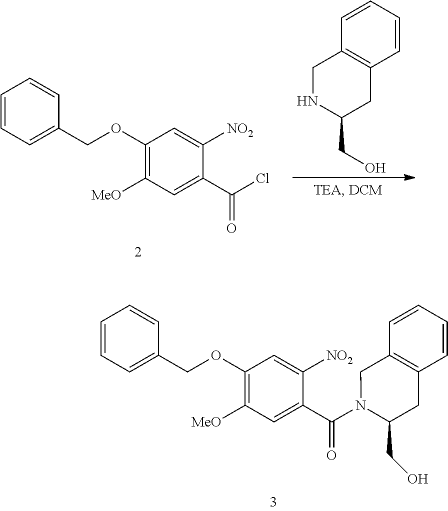 [SMILES: COc1cc(C(=O)Cl)c([N+](=O)[O-])cc1OCc1ccccc1 ; OC[C@@H]1Cc2ccccc2CN1 ; COc1cc(C(=O)N2Cc3ccccc3C[C@H]2CO)c([N+](=O)[O-])cc1OCc1ccccc1]

Step 2:

Compound 2 (5.3 g, 16.47 mmol) and (S)-(1,2,3,4-tetrahydroisoquinolin-3-yl)methanol (2.96 g, 18.12 mmol) were dissolved in DCM (47.1 mL). The reaction mixture was cooled to 0° C. and TEA (3.44 mL, 24.71 mmol) was added dropwise under Ar. The reaction mixture was then warmed to rt and was stirred overnight. The solution was concentrated and the crude product was purified by silica gel chromatography (EtOAc/hexanes, gradient, 0% to 80%) to obtain compound 3 (7.22 g, 16.10 mmol, 98% yield). LCMS=5.482 min (8 min method). Mass observed (ESI⁺): 449.25 (M+H).

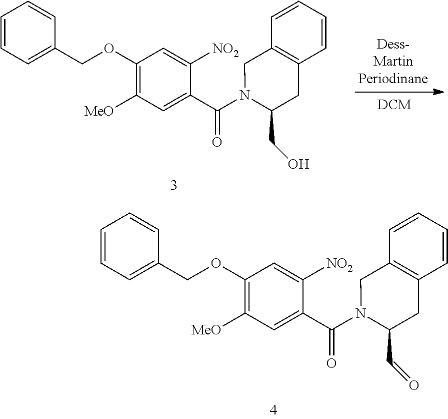 [SMILES: COc1cc(C(=O)N2Cc3ccccc3C[C@H]2CO)c([N+](=O)[O-])cc1OCc1ccccc1 ; COc1cc(C(=O)N2Cc3ccccc3C[C@H]2C=O)c([N+](=O)[O-])cc1OCc1ccccc1]

Step 3:

Compound 3 (6.0 g, 13.38 mmol) was dissolved in DCM (53.5 mL). Dess-Martin Periodinane (6.24 g, 14.72 mmol) was added slowly, portion-wise at 0° C. The reaction was then warmed to rt and was stirred for 3 h under Ar. The reaction was quenched with sat'd aq. sodium thiosulfate solution (20 mL), followed by a slow addition of sat'd NaHCO₃ (20 mL) and H₂O (20 mL). The mixture was stirred vigorously for ˜1 h. The layers were separated and the organic layer was washed with sat'd aq. sodium thiosulfate, sat'd NaHCO₃, brine, dried over Na₂SO₄, filtered and concentrated. The crude product was purified by silica gel chromatography (EtOAc/hexanes, 10% to 100%) to obtain compound 4 as pale yellow foam (5.45 g, 12.21 mmol, 91% yield). Mass observed (ESI⁺): 447.15 (M+H).

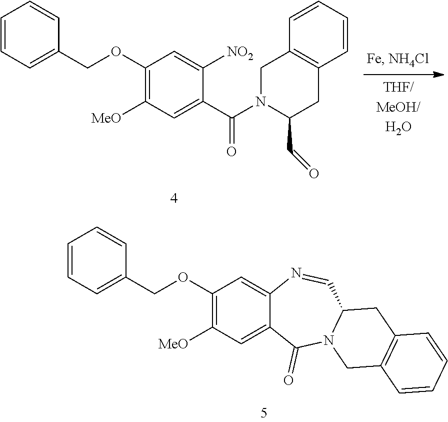 [SMILES: COc1cc(C(=O)N2Cc3ccccc3C[C@H]2C=O)c([N+](=O)[O-])cc1OCc1ccccc1 ; COc1cc2c(cc1OCc1ccccc1)N=C[C@@H]1Cc3ccccc3CN1C2=O]

Step 4:

Compound 4 (5.45 g, 12.21 mmol) was dissolved in THF (6.98 mL), methanol (34.9 mL) and water (6.98 mL) at rt. NH₄Cl (9.79 g, 183 mmol) was added, followed by iron powder (3.41 g, 61.0 mmol). The reaction was then heated reaction at 50° C. under Ar overnight. The reaction mixture was cooled to rt and was filtered through Celite. The cake was washed with DCM and the layers were separated. The organic layer was washed with brine, dried over Na₂SO₄, filtered and concentrated. The crude product was purified by silica gel chromatography (EtOAc/hexanes, 10% to 100%) to obtain compound 5 as a pale yellow foam (4.09 g, 10.26 mmol, 84% yield). ¹H NMR (400 MHz, CDCl₃): δ 7.55 (s, 1H), 7.46-7.43 (m, 3H), 7.39-7.34 (m, 3H), 7.33-7.29 (m, 4H), 6.85 (s, 1H), 5.20 (dd, 2H, J=12.3, 12.3 Hz), 5.00 (d, 1H, J=15.5 Hz), 4.56 (d, 1H, J=15.7 Hz), 3.97 (s, 3H), 3.88-4.00 (m, 1H), 3.26 (dd, 1H, J=15.4, 5.5 Hz), 3.14 (dd, 1H, J=15.3 4.2 Hz). LCMS=5.084 min (8 min method). Mass observed (ESI⁺): 399.15 (M+H).

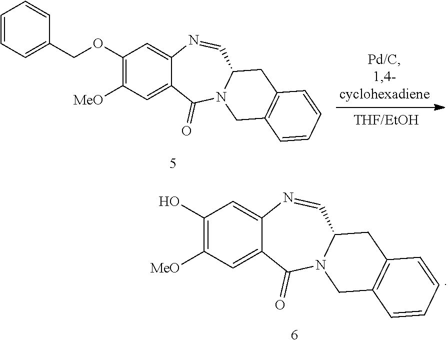 [SMILES: COc1cc2c(cc1OCc1ccccc1)N=C[C@@H]1Cc3ccccc3CN1C2=O ; COc1cc2c(cc1O)N=C[C@@H]1Cc3ccccc3CN1C2=O]

Step 5:

Compound 5 (4.09 g, 9.75 mmol) was dissolved in EtOH (48.8 mL) and THF (16.25 mL). The solution was degassed with Ar for 5 min. Pd/C (10%) (2.075 g, 1.950 mmol) was added slowly and the solution was degassed for 5 min. Cyclohexa-1,4-diene (7.38 mL, 78 mmol) was added and the reaction was stirred at rt with continuous bubbling of Ar overnight. The reaction mixture was filtered through Celite and was washed with MeOH/DCM (1:1, 50 mL), followed by MeOH (30 mL) and was concentrated. The crude product was purified by silica gel chromatography (EtOAc/hexanes, 0% 100%) to obtain THIQ-benzodiazepine monomer 6 (1.53 g, 4.27 mmol, 44% yield). LCMS=3.504 min (8 min method). Mass observed (ESI⁺): 309.15 (M+H), 327.15 (M+H₂O).

### Example 2. Synthesis of Compound 11

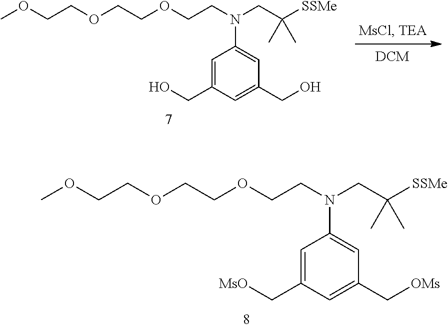

Step 1:

Compound 7 (100 mg, 0.231 mmol) was dissolved in DCM (1.54 mL) and was cooled to −10° C. (ice-salt bath) under Ar. TEA (80 μL, 0.577 mmol) was added, followed by a slow addition of MsC1 (41.3 μL, 0.530 mmol) and was stirred at −10° C. for 2 h. The reaction mixture was quenched with ice/water and was diluted with EtOAc and the layers were separated. The organic layer was washed with cold water (2×), dried over Na₂SO₄, filtered and concentrated to obtain dimesylate 8 (135 mg, 0.229 mmol, 99% yield). LCMS=5.829 min (8 min method). Mass observed (ESI⁺): 590.15 (M+H).

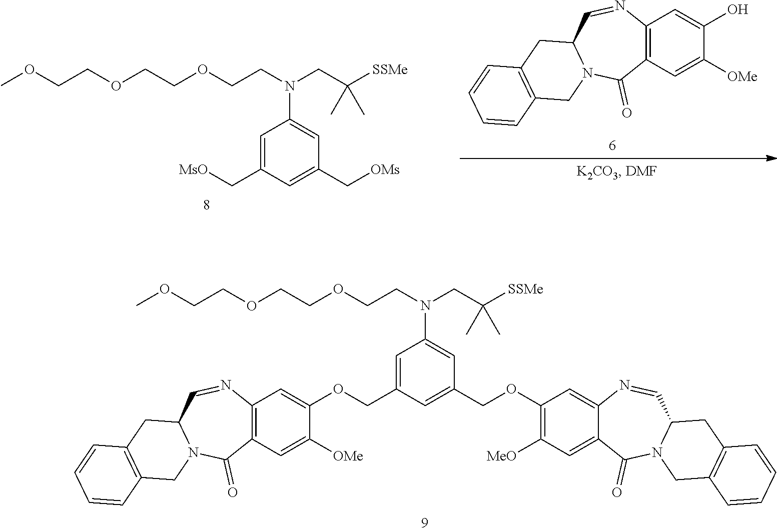

Step 2:

Compound 8 (135 mg, 0.229 mmol) and THIQ-benzodiazepine monomer 6 (148 mg, 0.481 mmol) were dissolved in DMF (1.14 mL). K₂CO₃ (79 mg, 0.572 mmol) was added at rt and was stirred under Ar overnight. The reaction mixture was diluted with EtOAc and was washed with water (2×), dried over Na₂SO₄, filtered and concentrated. The crude product was purified by silica gel chromatography (MeOH/DCM, 0% to 10%) to obtain compound 9 (132 mg, 0.130 mmol, 57% yield). LCMS=6.312 min (8 min method). Mass observed (ESI⁺): 1014.50 (M+H).

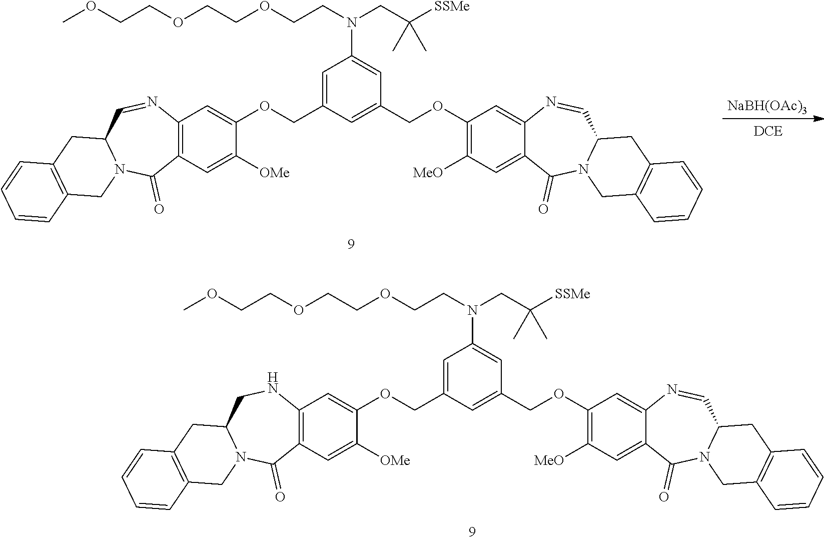

Step 3:

Compound 8 (130 mg, 0.090 mmol) was dissolved in DCE (897 μL). Sodium triacetoxyborohydride, STAB (17.11 mg, 0.081 mmol) was added at rt and was stirred for 1 h. The reaction mixture was diluted with EtOAc and a few drops of MeOH and was quenched with aq. citric acid solution. The layers were separated layers and the organic layer was washed with brine, dried over Na₂SO₄, filtered and concentrated. The crude reside was purified by RPHPLC (C18 column, CH₃CN/H₂O, gradient, 60% to 63%) to yield mono imine, 9 as a white fluffy solid (23 mg, 23% yield). LCMS (15 min method)=10.016 min. Mass observed (ESI⁺)=1016.6 (M+H).

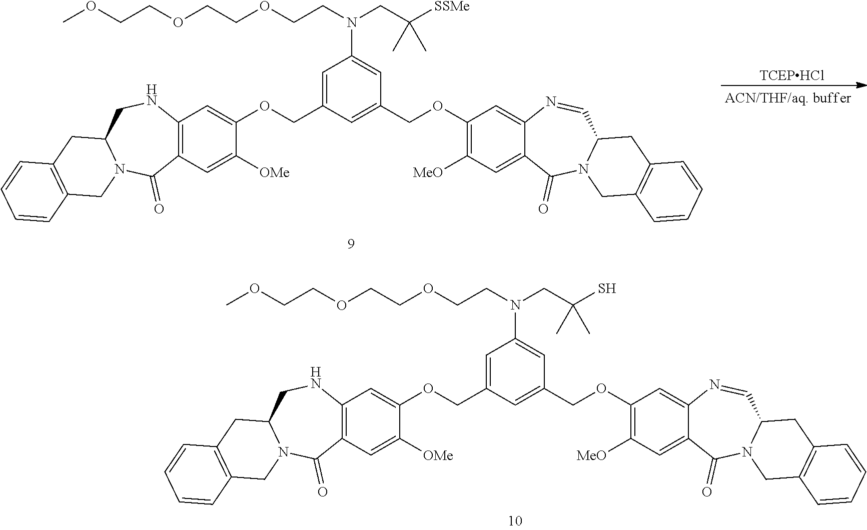

Step 4:

TCEP.HCl (15.23 mg, 0.053 mmol) was neutralized with water (˜100 μL) and sat'd aq. NaHCO₃ (˜150 VaL). 0.1 M NaH₂PO₄ buffer pH=6.5 (27 VaL) was added to the TCEP solution. In a separate flask, compound 9 (20 mg, 0.018 mmol) was suspended in CH₃CN (191 μL). The TCEP/buffer mixture (pH=6.5-7) was added to the solution, followed by the addition of methanol (136 VL) and was stirred at rt for 3 h. The reaction mixture was diluted with DCM and water. The layers were separated and the organic layer was washed with brine, dried over anhydrous Na₂SO₄, filtered and concentrated to give crude thiol 10, which was used in the next step without purification (14 mg, 0.014 mmol, 81% yield). LCMS (8 min method)=6.058 min. Mass observed (ESI⁺)=969.6 (M+H).

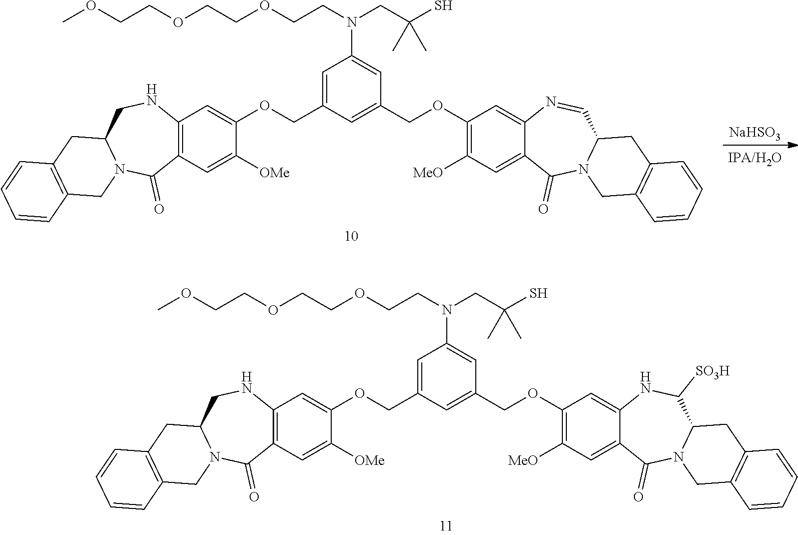

Step 5:

The crude thiol 10 (14 mg, 0.014 mmol) was suspended in 2-propanol (1.924 mL) and water (962 μL). NaHSO₃ (5.3 mg, 0.051 mmol) was added and the reaction was stirred at rt for 4.5 h. The clear solution was diluted with CH₃CN/H₂O (1:1, 15 mL) and was frozen and lyophilized. The resulting fluffy white powder was dissolved in CH₃CN/H₂O (1:1) and was purified by RPHPLC (C18 column, CH₃CN/H₂O, gradient, 25% to 40%) to give compound 11 as a white powder (5 mg, 4.75 μmol, 33% yield). LCMS (15 min method)=6.494 min. Mass observed=970.7 (ESI⁺, M-SO₃H+H), 1050.5 (ESI⁻, M−H).

### Example 3. Synthesis of Compound 17

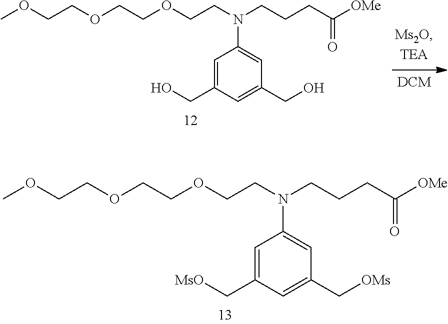 [SMILES: COCCOCCOCCN(CCCC(=O)OC)c1cc(CO)cc(CO)c1 ; COCCOCCOCCN(CCCC(=O)OC)c1cc(COS(C)(=O)=O)cc(COS(C)(=O)=O)c1]

Step 1:

Compound 12 (105 mg, 0.263 mmol) was dissolved in DCM (2.6 mL) and was cooled to −10° C. (acetone/ice bath) under Ar. TEA (183 μL, 1.314 mmol) was added, followed by Ms₂O (118, 0.657 mmol) and was stirred at ˜10° C. for 1 h. The reaction mixture was quenched with ice/water, diluted with EtOAc and the layers were separated. The organic layer was washed with cold water (2×), dried over Na₂SO₄, filtered and concentrated to obtain dimesylate 13 (128 mg, 0.223 mmol, 88% yield).

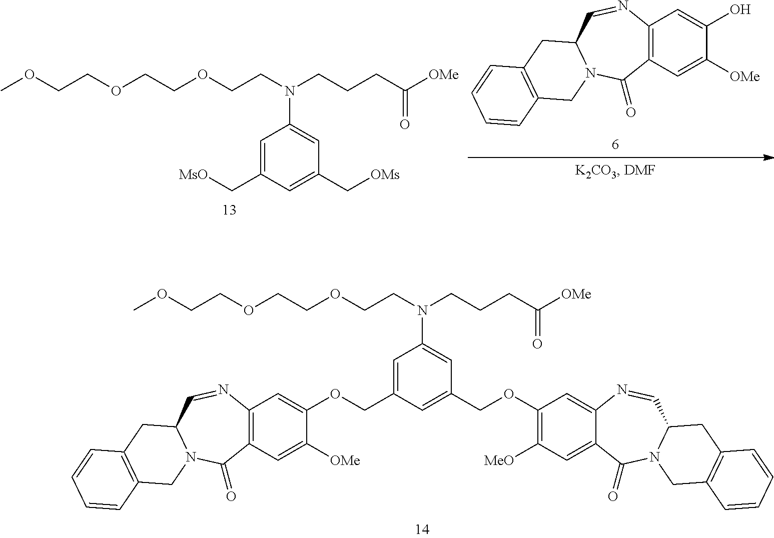

Step 2:

Compound 13 (100 mg, 0.180 mmol) and THIQ-benzodiazepine monomer 6 (122 mg, 0.396 mmol) were dissolved in DMF (1.8 mL). K₂CO₃ (62 mg, 0.45 mmol) was added at rt and was stirred under Ar overnight. Water was added to the reaction mixture. The resulting solid was filtered and was rinsed with water. The solid was redissolved in DCM and was washed with water, dried over MgSO₄, filtered and concentrated. The crude product was purified by silica gel chromatography (MeOH/DCM) to obtain compound 14 (80 mg, 0.065 mmol, 36% yield, 80% purity). LCMS=4.229 min (15 min method). Mass observed (ESI⁺): 980.8 (M+H).

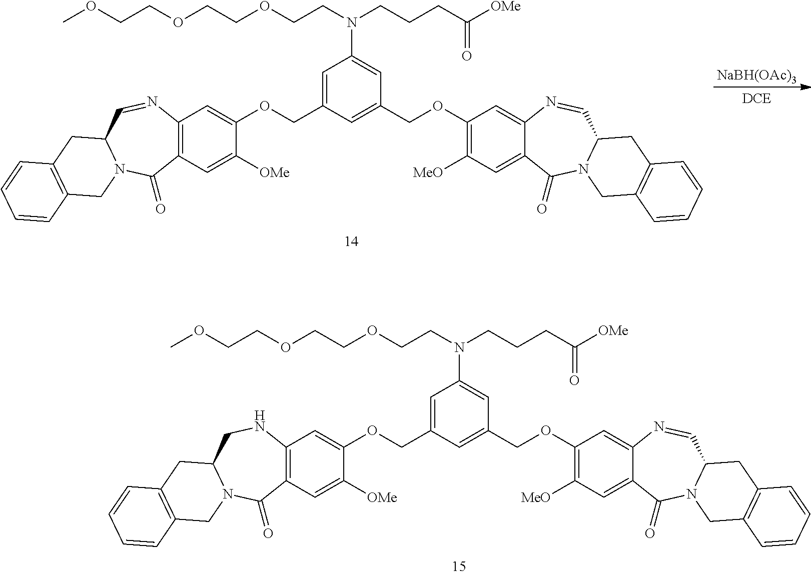 [SMILES: COCCOCCOCCN(CCCC(=O)OC)c1cc(COc2cc3c(cc2OC)C(=O)N2Cc4ccccc4C[C@H]2C=N3)cc(COc2cc3c(cc2OC)C(=O)N2Cc4ccccc4C[C@H]2C=N3)c1 ; COCCOCCOCCN(CCCC(=O)OC)c1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1 ; "NaBH(OAc)3"]

Step 3:

Compound 15 was synthesized similarly as compound 9 (page ##), by reacting compound 14 with STAB to obtain compound 15 (15 mg, 0.014 mmol, 31% yield). LCMS=4.983 min (15 min method). Mass observed (ESI⁺): 982.8 (M+H).

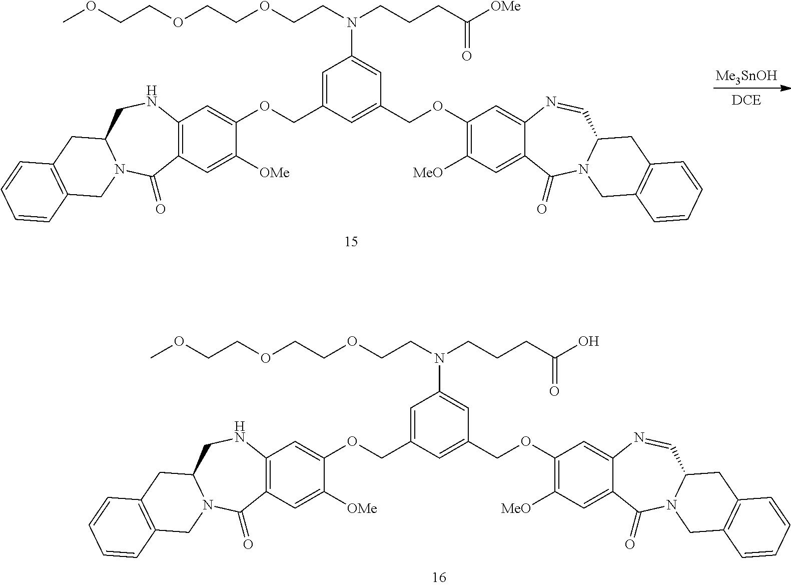 [SMILES: "Me3SnOH" ; COCCOCCOCCN(CCCC(=O)OC)c1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1 ; COCCOCCOCCN(CCCC(=O)O)c1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1]

Step 4:

Compound 15 (15 mg, 0.014 mmol) was dissolved in DCE (283 μL). Trimethyltin hydroxide (51 mg, 0.283 mmol) was added and the solution stirred overnight at 80° C. The reaction mixture was cooled to rt and was diluted with 10% MeOH/DCM and a few drops of 1 M aq. HCl solution until the aqueous phase turned pH ˜4-5. The layers were separated and the organic layer was washed with brine, dried over MgSO₄, filtered through Celite and concentrated. The crude product was passed through a silica plug with 10% MeOH/DCM to obtain 16 (7.5 mg, 6.74 μmol, 48% yield). LCMS=3.628 min (15 min method). Mass observed (ESI⁺): 968.8 (M+H).

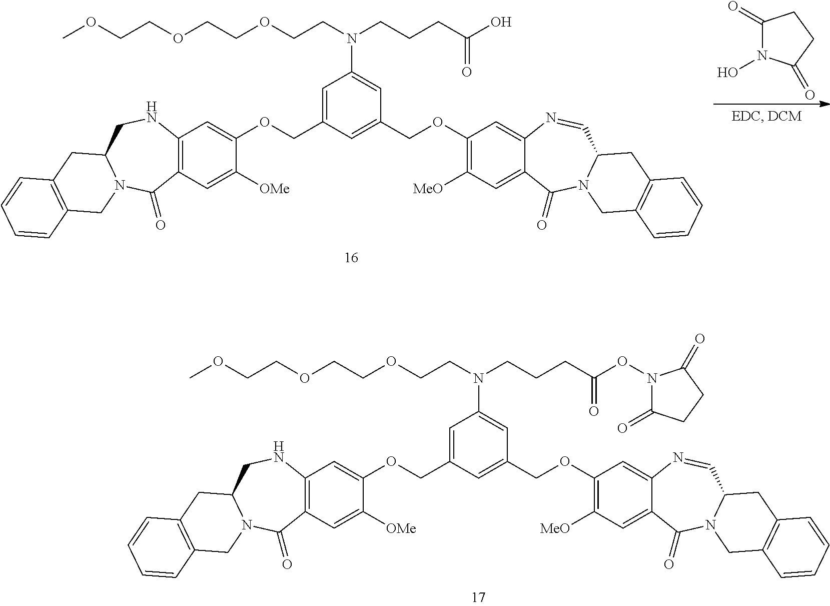 [SMILES: COCCOCCOCCN(CCCC(=O)O)c1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1 ; O=C1CCC(=O)N1O ; COCCOCCOCCN(CCCC(=O)ON1C(=O)CCC1=O)c1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1]

Step 5:

Compound 16 (7.5 mg, 6.74 μmol) was dissolved in DCM (0.35 mL). N-hydroxy succinimide (6.98 mg, 0.061 mmol) was added, followed by EDC.HCl (6.46 mg, 0.034 mmol) and was stirred at rt for 4 h. The reaction mixture was diluted with DCM and was washed with brine, dried over Na₂SO₄, filtered and concentrated. The crude product was purified by RPHPLC (C18 column, CH₃CN/H₂O, gradient) to give compound 17 as a white powder (1.1 mg, 0.826 μmol, 12% yield). LCMS (15 min method)=5.143 min. Mass observed=1065.8 (ESI⁺, M+H).

### Example 4. Synthesis of Compound 30

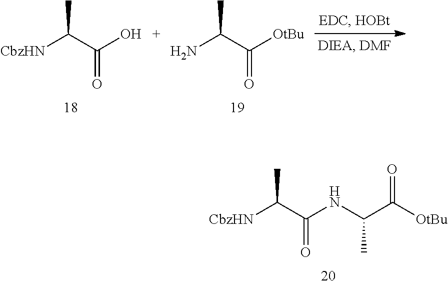

Step 1:

Z-Ala-OH, 18 (5.0 g, 22.40 mmol) and L-Ala-OtBu, 19 (4.48 g, 24.64 mmol) were dissolved in DMF (44.8 mL). EDC.HCl (4.72 g, 24.64 mmol) and HOBt (3.43 g, 22.40 mmol) were added to the reaction mixture, followed by DIPEA (9.75 mL, 56.0 mmol). The reaction was stirred under Ar at rt overnight. The reaction mixture was diluted with DCM and was washed with sat'd NaHCO₃, sat'd NH₄Cl, water and brine. The organic layer was dried over Na₂SO₄, filtered and concentrated. The crude residue was purified by silica gel flash chromatography (EtOAc/hexanes, gradient, 0% to 50%) to obtain compound 20 as a white solid (5.6 g, 15.90 mmol, 71% yield). ¹H NMR (400 MHz, CDCl₃): δ 7.39-7.34 (m, 5H), 6.54 (s, 1H) 5.28 (s, 1H), 5.15 (s, 2H), 4.47-4.43 (m, 1H), 4.48 (s, 1H), 1.49 (s, 9H), 1.42-1.37 (m, 6H).

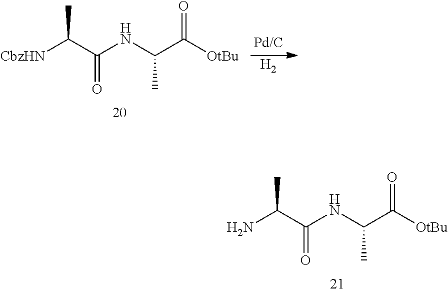 [SMILES: C[C@H](NC(=O)OCc1ccccc1)C(=O)N[C@@H](C)C(=O)OC(C)(C)C ; "H2" ; C[C@H](N)C(=O)N[C@@H](C)C(=O)OC(C)(C)C]

Step 2:

Compound 20 (6.7 g, 19.12 mmol) was dissolved in methanol (60.7 mL) and water (3.03 mL). The solution was purged with Ar for 5 min. Pd/C (wet, 10%) (1.017 g, 0.956 mmol) was added slowly. The reaction was stirred overnight under an atmosphere of hydrogen. The solution was filtered through Celite, rinsed with methanol and concentrated. The residue was coevaporated with methanol and acetonitrile and the resulting oil was placed on the high vacuum to give compound 21 which was taken onto the next step without purification (4.02 g, 18.57 mmol, 97% yield). ¹H NMR (400 MHz, CDCl₃): δ 7.78-7.63 (m, 1H), 4.49-4.42 (m, 1H), 3.55-3.50 (m, 1H), 1.73 (s, 2H), 1.48 (s, 9H), 1.39 (d, 3H, J=7.2 Hz), 1.36 (d, 3H, J=6.8 Hz).

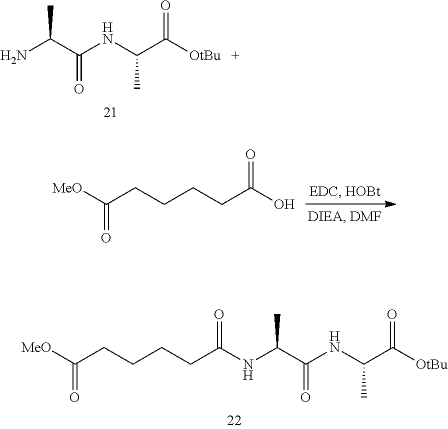 [SMILES: C[C@H](N)C(=O)N[C@@H](C)C(=O)OC(C)(C)C ; COC(=O)CCCCC(=O)O ; COC(=O)CCCCC(=O)N[C@@H](C)C(=O)N[C@@H](C)C(=O)OC(C)(C)C]

Step 3:

Compound 21 (4.02 g, 18.59 mmol) and mono methyladipate (3.03 mL, 20.45 mmol) were dissolved in DMF (62.0 mL). EDC.HCl (3.92 g, 20.45 mmol) and HOBt (2.85 g, 18.59 mmol) were added, followed by DIPEA (6.49 mL, 37.2 mmol). The mixture was stirred overnight at rt. The reaction mixture was diluted with DCM/MeOH (150 mL, 5:1) and was washed with sat'd NH₄Cl, sat'd NaHCO₃, brine, dried over Na₂SO₄, filtered and concentrated. The crude product was coevaporated with acetonitrile (5×), then pumped on high vacuum at 35° C. to give compound 22 (6.66 g, 100% yield). ¹H NMR (400 MHz, CDCl₃): δ 6.75 (d, 1H, J=6.8 Hz), 6.44 (d, 1H, J=6.8 Hz), 4.52-4.44 (m, 1H), 4.43-4.36 (m, 1H), 3.65 (s, 3H), 2.35-2.29 (m, 2H), 2.25-2.18 (m, 2H), 1.71-1.60 (m, 4H), 1.45 (s, 9H), 1.36 (t, 6H, J=6.0 Hz).

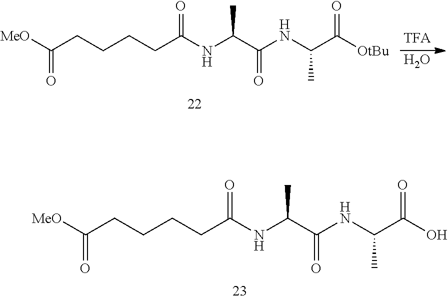 [SMILES: COC(=O)CCCCC(=O)N[C@@H](C)C(=O)N[C@@H](C)C(=O)OC(C)(C)C ; O ; COC(=O)CCCCC(=O)N[C@@H](C)C(=O)N[C@@H](C)C(=O)O]

Step 4:

Compound 22 (5.91 g, 16.5 mmol) was stirred in TFA (28.6 mL, 372 mmol) and deionized water (1.5 mL) at rt for 3 h. The reaction mixture was coevaporated with acetonitrile and placed on high vacuum to give compound 23 as a sticky solid (5.88 g, 100% yield). ¹H NMR (400 MHz, CDCl₃): δ 7.21 (d, 1H, J=6.8 Hz), 6.81 (d, 1H, J=7.6 Hz), 4.69-4.60 (m, 1H), 4.59-4.51 (m, 1H), 3.69 (s, 3H), 2.40-2.33 (m, 2H), 2.31-2.24 (m, 2H), 1.72-1.63 (m, 4H), 1.51-1.45 (m, 3H), 1.42-1.37 (m, 3H).

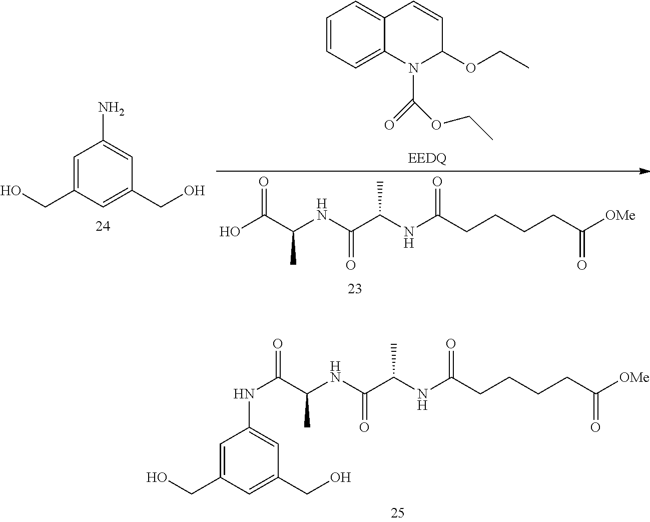 [SMILES: Nc1cc(CO)cc(CO)c1 ; COC(=O)CCCCC(=O)N[C@@H](C)C(=O)N[C@@H](C)C(=O)O ; CCOC(=O)N1c2ccccc2C=CC1OCC ; COC(=O)CCCCC(=O)N[C@@H](C)C(=O)N[C@@H](C)C(=O)Nc1cc(CO)cc(CO)c1]

Step 5:

Compound 23 (5.6 g, 18.52 mmol) was dissolved in DCM (118 mL) and methanol (58.8 mL). Diol 24 (2.70 g, 17.64 mmol) and EEDQ (8.72 g, 35.3 mmol) were added and the reaction was stirred at rt overnight. The reaction mixture was concentrated and ethyl acetate was added to the residue. The resulting slurry was filtered, washed with ethyl acetate and dried under vacuum/N₂ to give compound 25 as a white solid (2.79 g, 36% yield). ¹H NMR (400 MHz, DMSO-d6): δ 9.82 (s, 1H), 8.05, (d, 1H, J=9.2 Hz), 8.01 (d, 1H, J=7.2 Hz), 7.46 (s, 2H), 6.95 (3, 1H), 5.21-5.12 (m, 2H), 4.47-4.42 (m, 4H), 4.40-4.33 (m, 1H), 4.33-4.24 (m, 1H), 3.58 (s, 3H), 2.33-2.26 (m, 2H), 2.16-2.09 (m, 2H), 1.54-1.46 (m, 4H), 1.30 (d, 3H, J=7.2 Hz), 1.22 (d, 3H, J=4.4 Hz). LCMS=2.894 min (8 min method). Mass observed (ESI⁺): 438.20 (M+H).

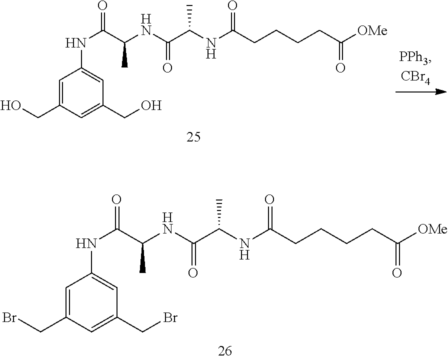 [SMILES: COC(=O)CCCCC(=O)N[C@@H](C)C(=O)N[C@@H](C)C(=O)Nc1cc(CO)cc(CO)c1 ; COC(=O)CCCCC(=O)N[C@@H](C)C(=O)N[C@@H](C)C(=O)Nc1cc(CBr)cc(CBr)c1]

Step 6:

Compound 25 (0.52 g, 1.189 mmol) and CBr₄ (1.183 g, 3.57 mmol) were dissolved in DMF (11.89 mL). PPh₃ (0.935 g, 3.57 mmol) was added and the reaction was stirred under Ar for 4 h. The reaction mixture was diluted with DCM/MeOH (10:1) and was washed with water, brine, dried over Na₂SO₄, filtered, and concentrated. The crude product was purified by silica gel chromatography (DCM/MeOH) to give compound 26 (262 mg, 39% yield). ¹H NMR (400 MHz, DMSO-d6): δ 10.01 (s, 1H), 8.11 (d, 1H, J=6.8 Hz), 8.03 (d, 1H, J=6.8 Hz), 7.67 (s, 2H), 7.21 (s, 1H), 4.70-4.64 (m, 4H), 4.40-4.32 (m, 1H), 4.31-4.23 (m, 1H), 3.58 (s, 3H), 2.34-2.26 (m, 2H), 2.18-2.10 (m, 2H), 1.55-1.45 (m, 4H), 1.31 (d, 3H, J=7.2 Hz), 1.21 (d, 3H, J=7.2 Hz). LCMS=4.939 min (8 min method). Mass observed (ESI⁺): 563.7 (M+H).

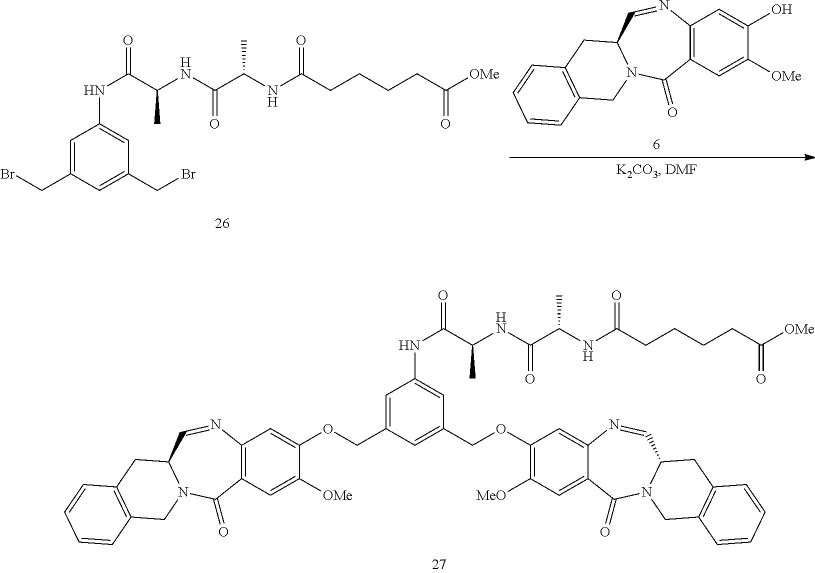

Step 7:

Compound 27 was prepared similarly as compound 14 (see pxx). Obtained compound 27 as a yellow solid after purification (118 mg, 0.089 mmol, 72% yield, 77% purity). LCMS=4.876 min (8 min method). Mass observed (ESI⁺): 1018.35 (M+H).

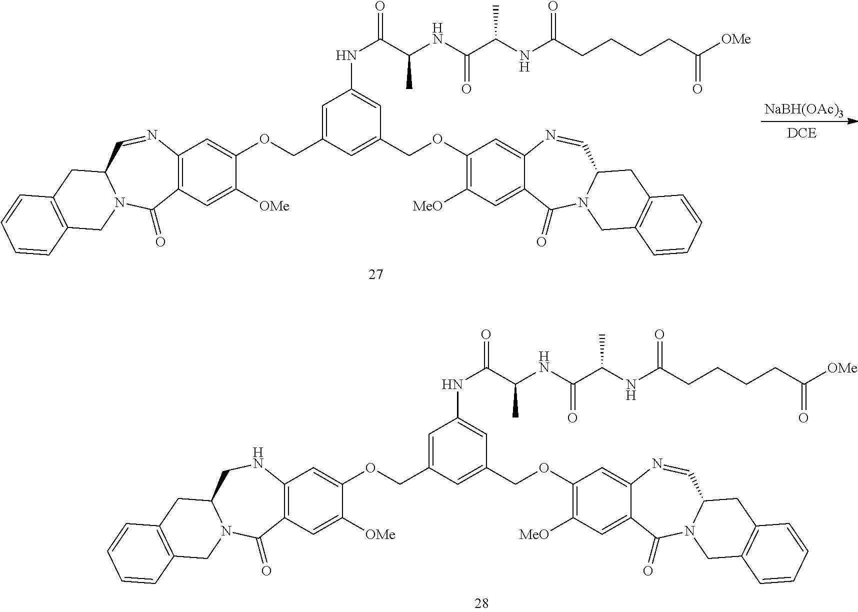 [SMILES: COC(=O)CCCCC(=O)N[C@@H](C)C(=O)N[C@@H](C)C(=O)Nc1cc(COc2cc3c(cc2OC)C(=O)N2Cc4ccccc4C[C@H]2C=N3)cc(COc2cc3c(cc2OC)C(=O)N2Cc4ccccc4C[C@H]2C=N3)c1 ; COC(=O)CCCCC(=O)N[C@@H](C)C(=O)N[C@@H](C)C(=O)Nc1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1 ; "NaBH(OAc)3"]

Step 8:

Compound 28 was prepared similarly as compound 9 (see pxx). Obtained 28 as a white solid after C18 purification (30 mg, 0.026 mmol, 30% yield). LCMS=5.021 min (8 min method). Mass observed (ESI⁺): 1020.30 (M+H).

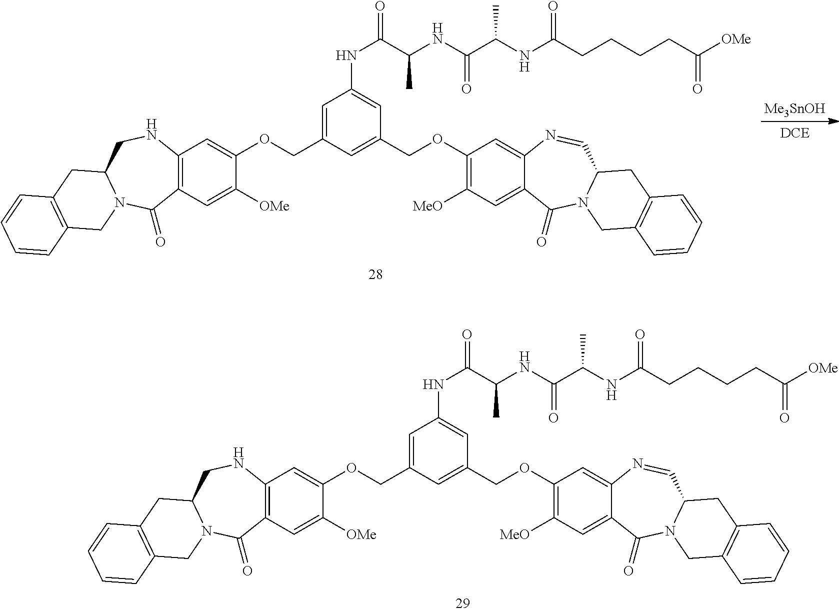 [SMILES: "Me3SnOH" ; COC(=O)CCCCC(=O)N[C@@H](C)C(=O)N[C@@H](C)C(=O)Nc1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1 ; COC(=O)CCCCC(=O)N[C@@H](C)C(=O)N[C@@H](C)C(=O)Nc1cc(COc2cc3c(cc2OC)C(=O)N2Cc4ccccc4C[C@H]2C=N3)cc(COc2cc3c(cc2OC)C(=O)N2Cc4ccccc4C[C@H]2CN3)c1]

Step 9:

Compound 29 was prepared similarly as compound 16 (see pxx). Obtained compound 29 as a yellow solid after silica plug (26 mg, 100% yield). HPLC=5.333 min (15 min method).

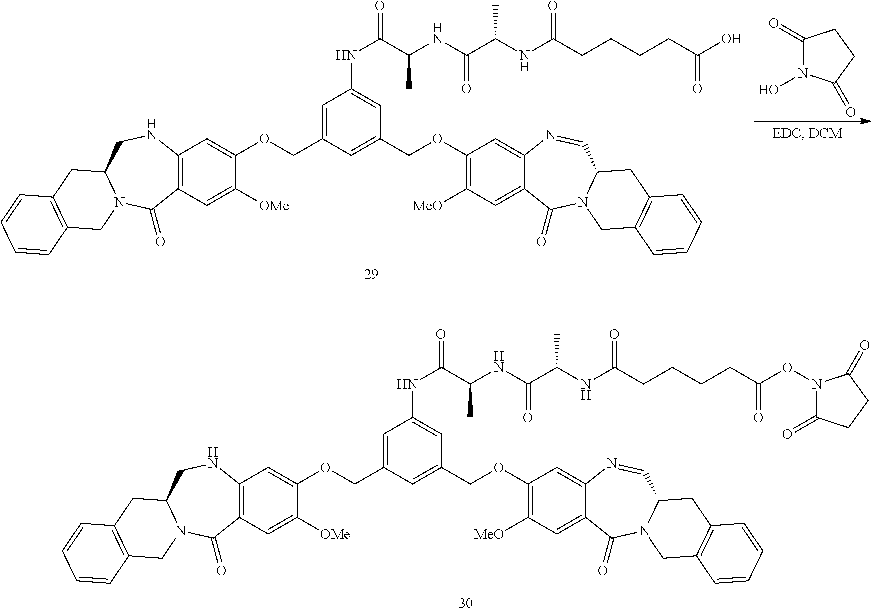 [SMILES: COc1cc2c(cc1OCc1cc(COc3cc4c(cc3OC)C(=O)N3Cc5ccccc5C[C@H]3CN4)cc(NC(=O)[C@H](C)NC(=O)[C@H](C)NC(=O)CCCCC(=O)O)c1)N=C[C@@H]1Cc3ccccc3CN1C2=O ; O=C1CCC(=O)N1O ; COc1cc2c(cc1OCc1cc(COc3cc4c(cc3OC)C(=O)N3Cc5ccccc5C[C@H]3CN4)cc(NC(=O)[C@H](C)NC(=O)[C@H](C)NC(=O)CCCCC(=O)ON3C(=O)CCC3=O)c1)N=C[C@@H]1Cc3ccccc3CN1C2=O]

Step 10:

Compound 30 was prepared similarly as compound 17 (see pxx). Obtained compound 30 as a white solid after C18 purification (9.3 mg, 8.43 μmol, 28% yield). LCMS=6.149 min (15 min method). Mass observed (ESI⁺): 1103.1 (M+H)

### Example 5. Preparation of Conjugates

a. Preparation of M9346A-Sulfo-SPDB-11 Conjugate

An in-situ mixture containing final concentrations of 3.9 mM compound 11 and 3 mM sulfo-SPDB linker in DMA containing 10 mM N,N-Diisopropylethyl amine (DIPEA) was incubated for 60 min before adding 8-fold excess of the resulting compound 11-sulfo-SPDB-NHS to a reaction containing 4 mg/ml M9346A antibody in 15 mM HEPES pH 8.5 (90:10 water: DMA). The solution was allowed to conjugate overnight at 25° C.

Post-reaction, the conjugate was purified and buffer exchanged into 100 mM Arginine, 20 mM Histidine, 2% sucrose, 0.01% Tween-20, 50 μM sodium bisulfite formulation buffer pH 6.2 using NAP desalting columns (Illustra Sephadex G-25 DNA Grade, GE Healthcare). Dialysis was performed in the same buffer over night at 4° C. utilizing Slide-a-Lyzer dialysis cassettes (ThermoScientific 10,000 MWCO).

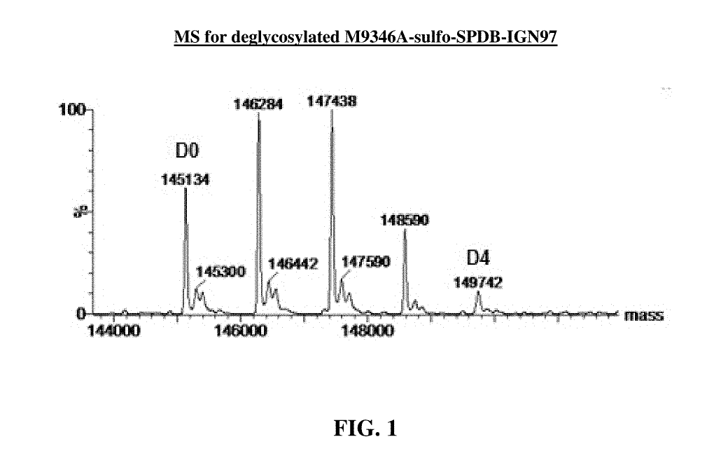

The purified conjugate was found to have an average of 2.5 compound 11 molecules linked per antibody (by SEC using molar extinction coefficients E₃₁₇ nm=9,554 cm⁻¹M⁻¹ and ε₂₈₀ nm=30, 115 cm⁻¹M⁻¹ for IGN97, and ε_{280 nm}=201,400 cm⁻¹M⁻¹ for M9346A antibody), 97.3% monomer (by size exclusion chromatography), and a final protein concentration of 0.32 mg/ml. Mass spectrum of the deglycosylated conjugate is shown in FIG. 1.

b. Preparation of M9346A-17Conjugate

A reaction containing 2.0 mg/mL M9346A antibody and 5 molar equivalents compound 17 (pretreated with 5-fold excess of sodium bisulfite in 90:10 DMA:water) in 50 mM HEPES (4-(2-hydroxyethyl)-1-piperazine ethanesulfonic acid) pH 8.5 buffer and 15% v/v DMA (N,N-Dimethylacetamide) cosolvent was allowed to conjugate for 6 hours at 25° C.

Post-reaction, the conjugate was purified and buffer exchanged into 250 mM Glycine, 10 mM Histidine, 1% sucrose, 0.01% Tween-20, 50 μM sodium bisulfite formulation buffer pH 6.2 using NAP desalting columns (Illustra Sephadex G-25 DNA Grade, GE Healthcare). Dialysis was performed in the same buffer for 20 hours at 4° C. utilizing Slide-a-Lyzer dialysis cassettes (ThermoScientific 20,000 MWCO).

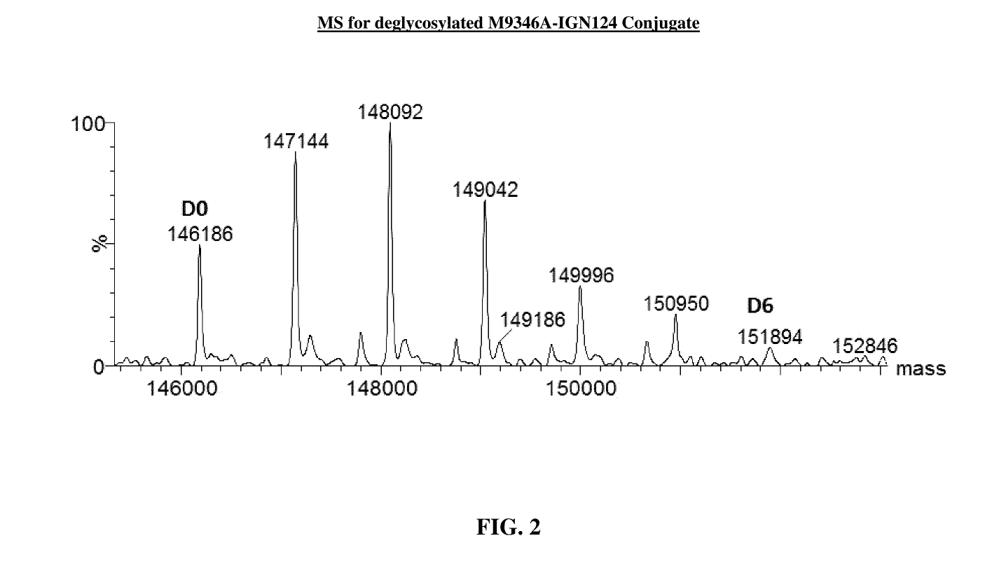

The purified conjugate was found to have an average of 2.8 molecules of compound 17 linked per antibody (by UV-Vis using molar extinction coefficients ε_{317 nm}=9554 cm⁻¹M⁻¹ and ε_{280 nm}=30, 115 cm⁻¹M⁻¹ for IGN124, and ε_{280 nm}=201,400 cm⁻¹M⁻¹ for M9346A antibody), 96% monomer (by size exclusion chromatography), <0.1% unconjugated compound 17 (by acetone precipitation, reverse-phase HPLC analysis) and a final protein concentration of 1.2 mg/ml. The conjugated antibody was found to be >95% intact by gel chip analysis. Mass spectrum of the deglycosylated conjugate is shown in FIG. 2.

c. Preparation of M9346A-30 Conjugate

A reaction containing 2.0 mg/mL M9346A antibody and 5 molar equivalents compound 30 (pretreated with 5-fold excess of sodium bisulfite in 90:10 DMA:water) in 50 mM HEPES (4-(2-hydroxyethyl)-1-piperazine ethanesulfonic acid) pH 8.5 buffer and 15% v/v DMA (N,N-Dimethylacetamide) cosolvent was allowed to conjugate for 6 hours at 25° C.

Post-reaction, the conjugate was purified and buffer exchanged into 250 mM Glycine, 10 mM Histidine, 1% sucrose, 0.01% Tween-20, 50 μM sodium bisulfite formulation buffer pH 6.2 using NAP desalting columns (Illustra Sephadex G-25 DNA Grade, GE Healthcare). Dialysis was performed in the same buffer for 20 hours at 4° C. utilizing Slide-a-Lyzer dialysis cassettes (ThermoScientific 20,000 MWCO).

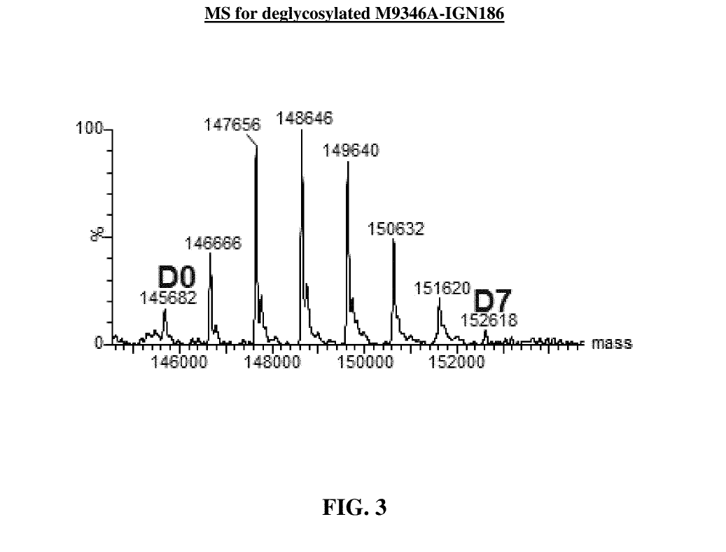

The purified conjugate was found to have an average of 3.0 molecules of compound 30 linked per antibody (by UV-Vis using molar extinction coefficients E_{318 nm}=14,000 cm⁻¹M⁻¹ and ε_{280 nm}=21,000 cm⁻¹M⁻¹ for compound 30, and ε_{280 nm}=201,400 cm⁻¹M⁻¹ for M9346A antibody), 93% monomer (by size exclusion chromatography), <1% unconjugated IGN186 (by acetone precipitation, reverse-phase HPLC analysis) and a final protein concentration of 1.25 mg/ml. The conjugated antibody was found to be >95% intact by gel chip analysis. Mass spectrum of the deglycosylated conjugate is shown in FIG. 3.

### Example 6. Binding Assay (Flow Cytometry)

T47D cells (breat epithelial cancer, ATCC) were maintained and plated for the binding experiments in media recommended by the manufacturer. 20,000 T47D cells per well in the 96-well round bottom plate were incubated for 2 hours at 4° C. with unconjugated antibody or conjugates diluted to various concentrations in FACS buffer (0.01 M PBS, pH 7.4 (Life Technoliges) supplemented with 0.5% BSA (Boston BioProducts)). The cells were then washed in cold FACS buffer, stained with FITC-labeled Goat Anti-Human-IgG-Fcγ specific antibody (Jackson ImmunoResearch) for 1 hr at 4° C., washed with the cold FACS buffer, fixed in 1% formaldehyde/0.01 M PBS overnight and then read using a FACS Calibur (BD Biosciences). Binding curves and EC₅₀ were generated using a sigmoidal dose-response nonlinear regression curve fit (GraphPad Software Inc.).

TABLE 1
EC₅₀ values for in vitro flow cytometry binding assays

| | | Un-conjugated |
|---|---|---|
| | | Antibody |
| | Conjugate | control* |
| M9346A-sulfo-SPDB-11 | 3e-10M | 2e-10M |
| M9346A-17 | 5e-10M | 5e-10M |
| M9346A-30 | 3e-10M | 1e-10M |

*The EC₅₀ values for each conjugate and the unconjugated antibody control were generated in independent experiments which might explain slight variability of the unconjugated control antibody EC₅₀ values.

### Example 7. Cytotoxicity Assay

Following cell lines were used for the study: KB (cervical carcinoma, ATCC), NCI-H2110 (Non Small Cell Lung Carcinoma, ATCC) and T47D (breast epithelial cancer, ATCC). The cells were maintained and plated for the cytotox experiments in media recommended by the manufacturers. Cells were plated in the 96-well flat bottom plates at a seeding density of 1,000 cells per well (KB) or 2,000 cell per well (NCI H2110 and T47D). Conjugates were diluted in RPMI-1640 (Life Technologies) supplemented with heat-inactivated 10% FBS (Life Technologies) and 0.1 mg/ml gentamycin (Life Technologies), and added to the plated cells. The plates were incubated at 37° C., 6% CO₂ for either 4 days (T47D cells) or 5 days (KB, NCI H2110 cells). Alamar blue assay (Invitrogen) was used to determine viability of T47D cells, and WST-8 assay (Donjindo Molecular Technologies, Inc.) was applied for KB and NCI H21110 cells. The assays were performed in accordance with the manufacturer's protocols. Killing curves and IC₅₀ were generated using a sigmoidal dose-response nonlinear regression curve fit (GraphPad Software Inc.) Following cell lines were used for the study: KB (cervical carcinoma, ATCC), NCI-H2110 (Non Small Cell Lung Carcinoma, ATCC) and T47D (breast epithelial cancer, ATCC). The cells were maintained and plated for the cytotox experiments in media recommended by the manufacturer. Cells were plated in the 96-well flat bottom plates at a seeding density of 1,000 cells per well (KB) or 2,000 cell per well (NCI H2110 and T47D). Conjugates were diluted in RPMI-1640 (Life Technologies) supplemented with heat-inactivated 10% FBS (Life Technologies) and 0.1 mg/ml gentamycin (Life Technologies), and added to the plated cells. To determine specificity of cytotoxic activity of the conjugates an excess of unconjugated antibody was added to a separate set of diluted conjugates (+block samples, IC50 table). The plates were incubated at 37° C., 6% CO2 for either 4 days (T47D cells) or 5 days (KB, NCI H2110 cells). Alamar blue assay (Invitrogen) was used to determine viability of T47D cells, and WST-8 assay (Donjindo Molecular Technologies, Inc.) was applied for KB and NCI H21110 cells. The assays were performed in accordance with the manufacturer's protocols. Killing curves and IC50 were generated using a sigmoidal dose-response nonlinear regression curve fit (GraphPad Software Inc.).

TABLE 2
IC₅₀ values for in vitro cytotocity of the conjugates

| | KB | KB | H2110 | H2110 | T47D | T47D |
|---|---|---|---|---|---|---|
| | −block | +block | −block | +block | −block | +block |
| M9346A-sulfo-SPDB-11 | 7e−11 M | 2e−9 M | ND | ND | ND | ND |
| M9346A-17 | 1e−11 M | 3e−9 M | 1e−10 M | 6e−9 M | ND | ND |
| M9346A-30 | 3e−12 M | 1e−9 M | 2e−11 M | 7e−9 M | 1e−11 M | 2e−8 M |

ND = Not determined

### Example 8. Bystander Cytotoxicity Assay

A mixed culture of FRα-positive cells 300-19 transfected with human FRa and FRα-negative cells 300-19 was exposed to conjugates at concentrations that are not toxic for the negative cells but highly toxic for the receptor-positive cells (killing 100% of the cells). Cells were incubated for 4 days, and the inhibition of cell proliferation was determined by Cell Titer Glo (Promega) according to the manufacturer's protocol.

In Vitro Bystander Activity in 300.19 Cell System, −/+FRα

| | Activity | Level |
|---|---|---|
| M9346A-sulfo- | ND | ND |
| SPDB-11 | | |
| M9346A-17 | no | − |
| M9346A-30 | yes | + |

ND = Not determined

### Example 9. In Vivo Tolerability Study

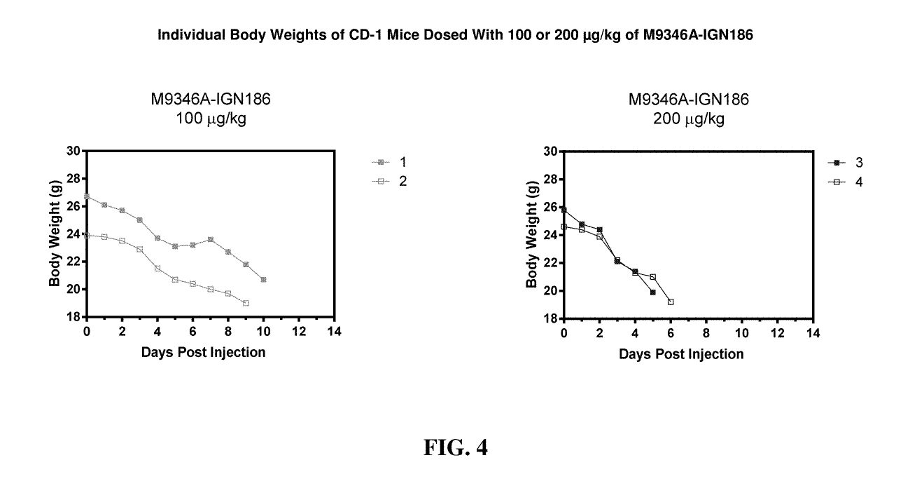

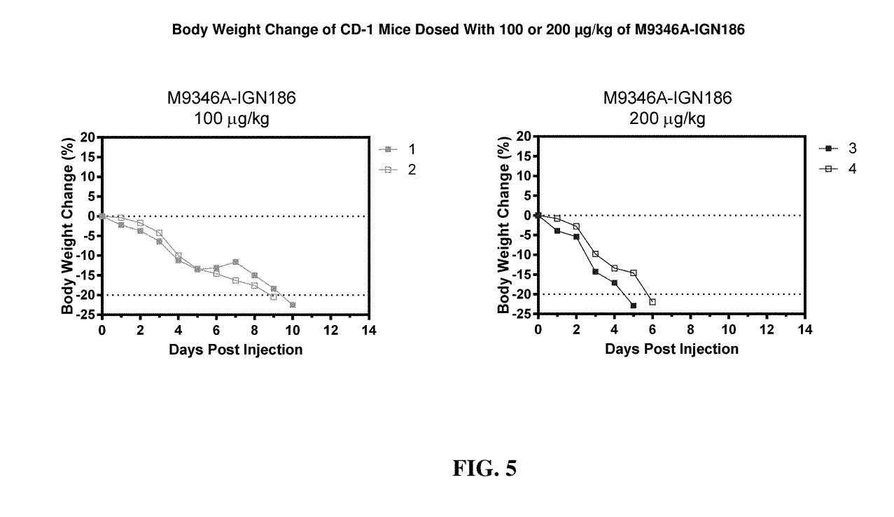

The tolerability of M9346A conjugates was investigated in female CD-1 mice. Animals were observed for seven days prior to study initiation and found to be free of disease or illness. The mice were administered a single i.v. injection of the M9346A-30 conjugate and the animals were monitored daily for body weight loss, morbidity or mortality. The M9346A-30 conjugate was not tolerated at a dose of 100 μg/kg or 200 μg/kg. At 100 μg/kg, the M9346A-30 conjugate caused ½ mice to exceed 20% body weight loss on day 9 post dosing and the other exceed 20% body weight loss on day 10 post dosing. At 200 μg/kg, the M9346A-30 conjugate caused ½ mice to exceed 20% body weight loss on day 5 post dosing and the other exceed 20% body weight loss on day 6 post dosing. Individual body weight and body weight change for the mice are shown in FIGS. 4 and 5.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

1. A compound represented by the following formula: 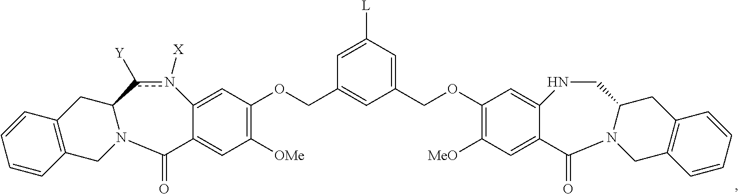
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;
L is represented by the following formula:
—NR₅—P—C(═O)—W-J (L1);
—NR₅—P—C(═O)—W—S—Z^{s} (L2);
—N(R^{e′})—W—S—Z^{s} (L3);
—N(R^{e})—C(═O)—W—S—Z^{s} (L4); or
—N(R^{e′})—W-J (L5);
R₅, for each occurrence, is independently H or a (C₁-C₃)alkyl;
W is a spacer unit;
J is a reactive moiety capable of forming a covalent bond with a cell-binding agent;
R^{e} is H or a (C₁-C₃)alkyl;
R^{e′} is —(CH₂—CH₂—O)ₙ—R^{k};
n is an integer from 2 to 6;
R^{k} is H or Me;
Z^{s} is H, —SR^{d}, —C(═O)R^{d1} or a bifunctional linker having a reactive moiety capable of forming a covalent bond with a cell-binding agent;
R^{d} is a (C₁-C₆)alkyl or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and
R^{d1} is a (C₁-C₆)alkyl.

2-3. (canceled)

4. The compound of claim 1, wherein the compound is represented by the following formula: 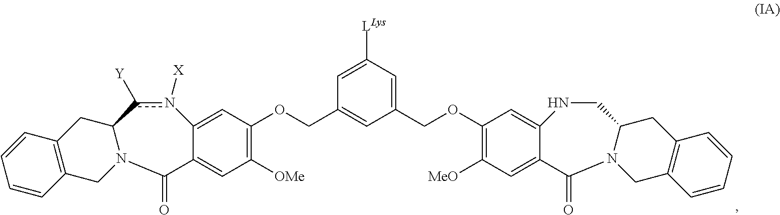
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;
L^{Lys} is represented by the following formula:
—NR₅—P—C(═O)—(CR^{a}R^{b})ₘ-J^{Lys} (L1);
—NR₅—P—C(═O)—(CR^{a}R^{b})ₘ—S—Z^{s} (L2);
—N(R^{e})—C(═O)—R^{x1}—S—Z^{s} (L3);
—N(R^{e′})—R^{x2}—S—Z^{s} (L4);
—N(R^{e′})—R^{x3}-J^{Lys} (L5);
R₅ is —H or a (C₁-C₃)alkyl;
P is an amino acid residue or a peptide containing between 2 to 20 amino acid residues;
Rₐ and R_{b}, for each occurrence, are each independently —H, (C₁-C₃)alkyl, or a charged substituent or an ionizable group Q;
m is an integer from 1 to 6;
R^{x1}, R^{x2} and R^{x3} are each independently a (C₁-C₆)alkyl;
R^{e} is —H or a (C₁-C₆)alkyl;
R^{e′} is —(CH₂—CH₂—O)ₙ—R^{k};
n is an integer from 2 to 6;
R^{k} is —H or -Me;
J^{Lys} is —COOR^{c} or —C(═O)E, wherein R^{c} is H or a (C₁-C₃)alkyl; and —C(═O)E represents a reactive ester;
Z^{s} is H, —SR^{d}, —C(═O)R^{d1} or is selected from any one of the following formulae: 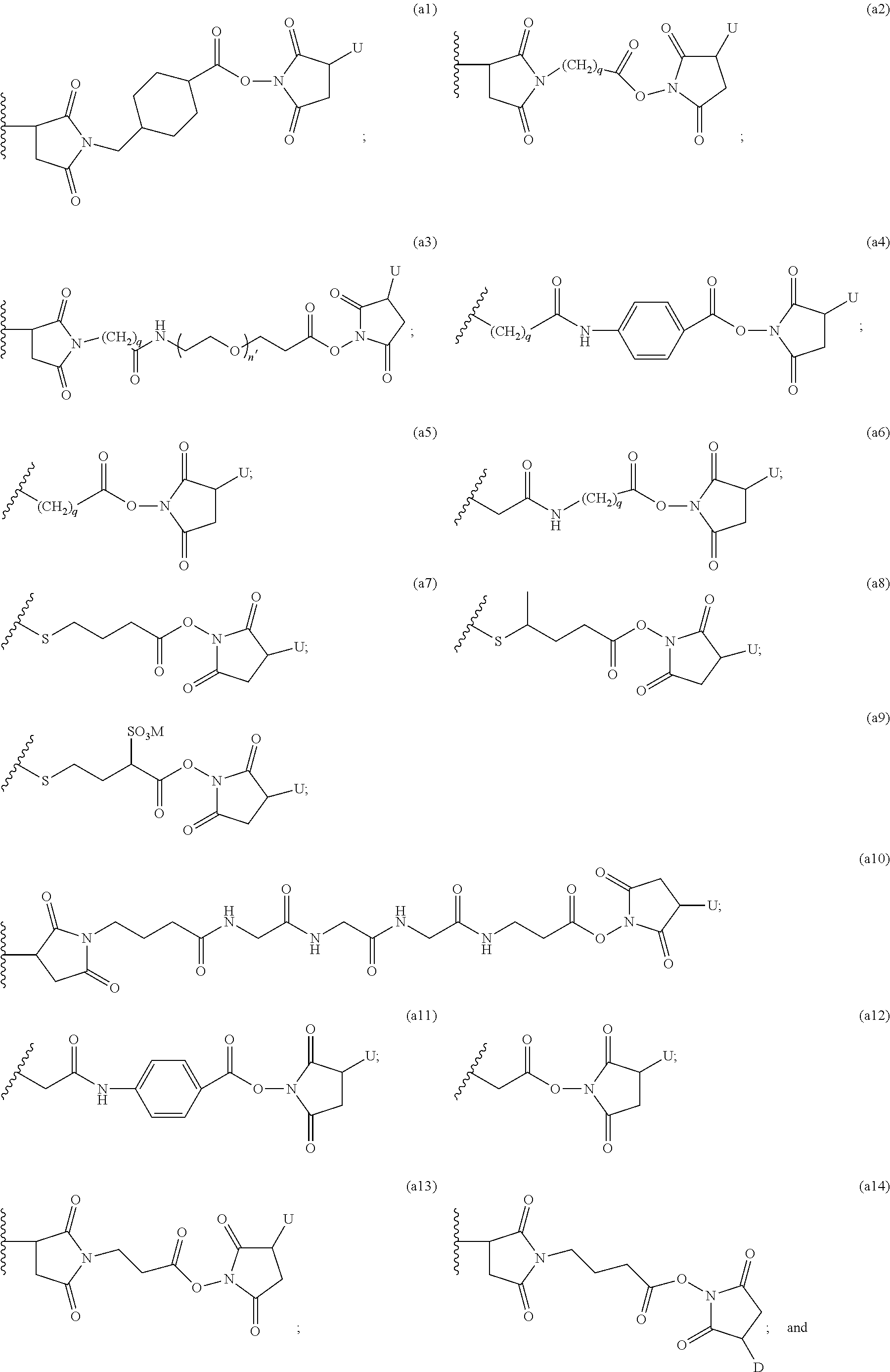 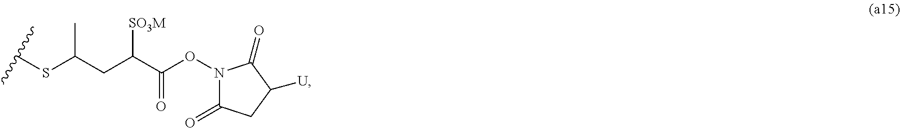
q is an integer from 1 to 5;
n′ is an integer from 2 to 6;
U is H or SO₃M;
M is H or a pharmaceutically acceptable cation;
R^{d} is a (C₁-C₆)alkyl or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and
R^{d1} is a (C₁-C₆)alkyl.

5. The compound of claim 4, wherein P is a peptide containing 2 to 5 amino acid residues.

6. The compound of claim 4, wherein P is selected from Gly-Gly-Gly, Ala-Val, Val-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO: 1), P-Ala-Leu-Ala-Leu (SEQ ID NO: 2), Gly-Phe-Leu-Gly (SEQ ID NO: 3), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Gln-Val, Asn-Ala, Gln-Phe and Gln-Ala.

7. (canceled)

8. The compound of claim 4, wherein R₅ is H or Me or.

9. The compound of claim 4, wherein Q is —SO₃M.

10. The compound of claim 4, wherein R^{a} and R^{b}, for each occurrence, are independently H or Me.

11. The compound of claim 4, wherein J^{Lys} is a reactive ester selected from the group consisting of N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

12. (canceled)

13. The compound of claim 4, wherein Z^{s} is H or —SR^{d}, wherein R^{d} is a (C₁-C₃)alkyl, pyridyl or nitropyridyl (e.g., 4-nitropyridyl).

14. The compound of claim 4, wherein Z^{s} is selected from any one of the following formulae:

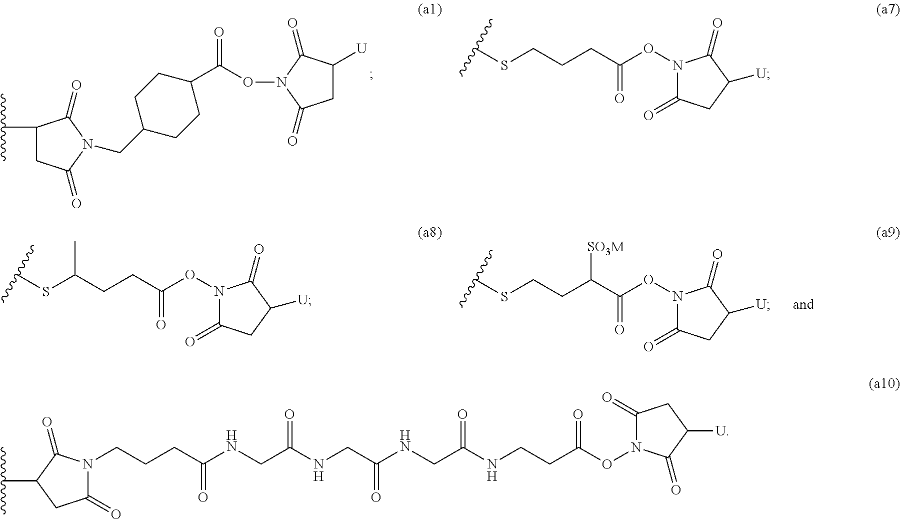

15. The compound of claim 1, wherein the double line [symbol] between N and C represents a double bond, X is absent and Y is —H or the double line [symbol] between N and C represents a single bond, X is H and Y is —SO₃M.

16. (canceled)

17. The compound of claim 4, wherein:
the double line [symbol] between N and C represents a single bond or a double bond, provided that when it is a double bond, X is absent and Y is —H; and when it is a single bond, X is —H, and Y is —SO₃M;
M is H, Na⁺ or K⁺;
L^{Lys} is represented by the following formula:
—NR₅—P—C(═O)—(CR^{a}R^{b})ₘ-J^{Lys} (L1);
wherein:
R^{a} and R^{b} are both —H;
m is 3 to 5;
P is Ala-Ala, Ala-D-Ala, D-Ala-Ala, or D-Ala-D-Ala;
R₅ is H or Me; and
J^{Lys} is N-hydroxysuccinimide ester or N-hydroxy sulfosuccinimide ester.

18. The compound of claim 4, wherein:
the double line [symbol] between N and C represents a single bond or a double bond, provided that when it is a double bond, X is absent and Y is —H; and when it is a single bond, X is —H, and Y is —SO₃M;
M is H, Na⁺ or K⁺;
L^{Lys} is represented by the following formula:
—NR₅—P—C(═O)—(CR^{a}R^{b})ₘ—S—Z^{s} (L2),
wherein:
—(CR^{a}R^{b})ₘ— is —(CH₂)ₚ—(CR^{f}R^{g})—, wherein R^{f} and R^{g} are each independently —H or -Me; and p is 0, 1, 2 or 3;
P is Ala-Ala, Ala-D-Ala, D-Ala-Ala, or D-Ala-D-Ala;
R is H or Me;
Z^{s} is H, —SR^{d} or is represented by formula (a1), (a7), (a8), (a9) or (a10); and
R^{d} is a (C₁-C₃)alkyl, pyridyl or nitropyridyl (e.g., 4-nitropyridyl).

19. The compound of claim 4, wherein:
the double line [symbol] between N and C represents a single bond or a double bond, provided that when it is a double bond, X is absent and Y is —H; and when it is a single bond, X is —H, and Y is —SO₃M;
M is H, Na⁺ or K⁺;
L is represented by the following formula:
—N(R^{e})—C(═O)—Rx-S—Z^{s} (L3);
wherein:
R^{e} is H or Me;
R^{x1} is —(CH₂)ₚ—(CR^{f}R^{g})—, wherein R^{f} and R^{g} are each independently —H or -Me; and p is 0, 1, 2 or 3;
Z^{s} is H, —SR^{d} or is represented by formula (a1), (a7), (a8), (a9) or (a10); and
R^{d} is a (C₁-C₃)alkyl, pyridyl or nitropyridyl (e.g., 4-nitropyridyl).

20. The compound of claim 4, wherein:
the double line [symbol] between N and C represents a single bond or a double bond, provided that when it is a double bond, X is absent and Y is —H; and when it is a single bond, X is —H, and Y is —SO₃M;
M is H, Na⁺ or K⁺;
L^{Lys} is represented by the following formula:
—N(R^{e′})—R^{x2}—S—Z^{s} (L4);
wherein:
R^{x2} is —(CH₂)ₚ—(CR^{f}R^{g})—, wherein R^{f} and R^{g} are each independently —H or -Me; and p is 0, 1, 2 or 3;
R^{e′} is —(CH₂—CH₂—O)ₙ—R^{k};
R^{k} is Me;
Z^{s} is H, —SR^{d} or is represented by formula (a1), (a7), (a8), (a9) or (a10); and R^{d} is a (C₁-C₃)alkyl, pyridyl or nitropyridyl (e.g., 4-nitropyridyl).

21. The compound of claim 4, wherein:
the double line [symbol] between N and C represents a single bond or a double bond, provided that when it is a double bond, X is absent and Y is —H; and when it is a single bond, X is —H, and Y is —SO₃M;
M is H, Na⁺ or K⁺;
L^{Lys} is represented by the following formula:
—N(R^{e′})R^{x3}-J^{Lys} (L5);
wherein:
R^{e′} is —(CH₂—CH₂—O)ₙ—R^{k};
R^{k} is Me;
R^{x3} is —(CR^{a}R^{b})ₘ—
R^{a} and R^{b} are both —H;
m is 3 to 5; and
J^{Lys} is N-hydroxysuccinimide ester or N-hydroxy sulfosuccinimide ester.

22. The compound of claim 4, wherein the compound is represented by any one of the following formula: 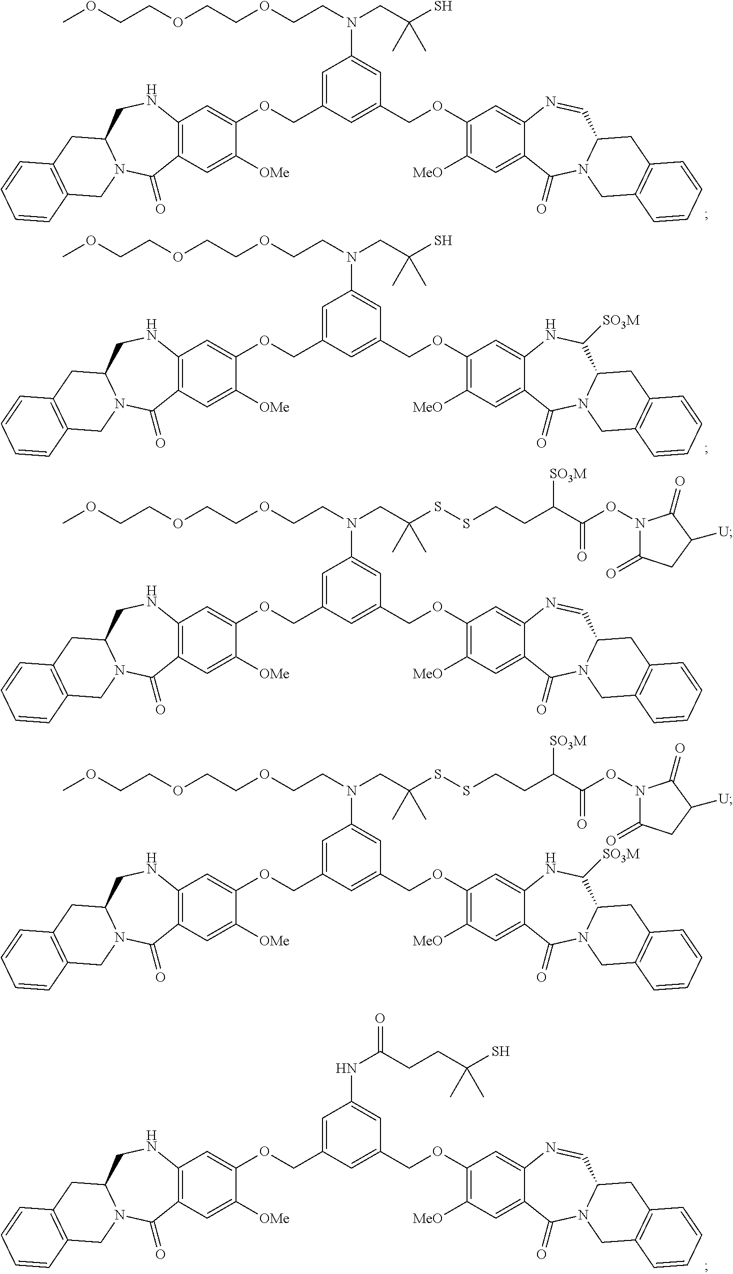 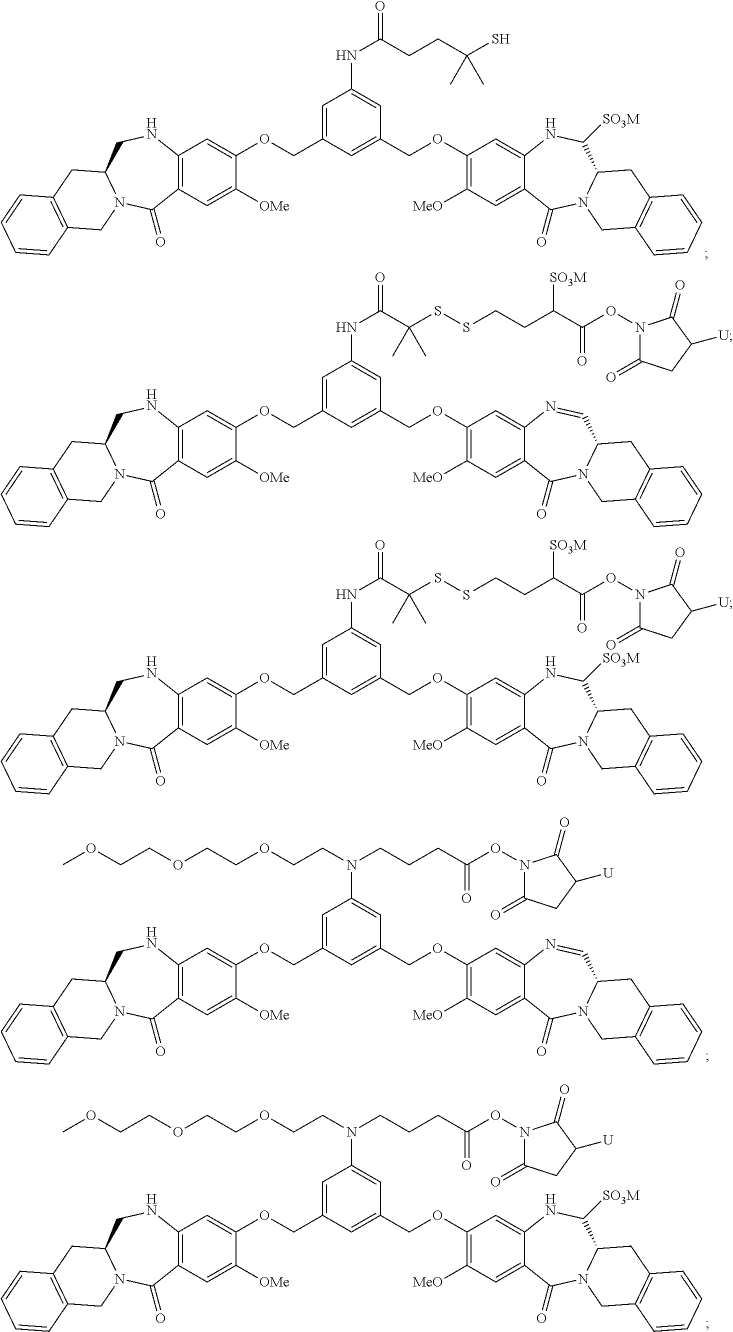 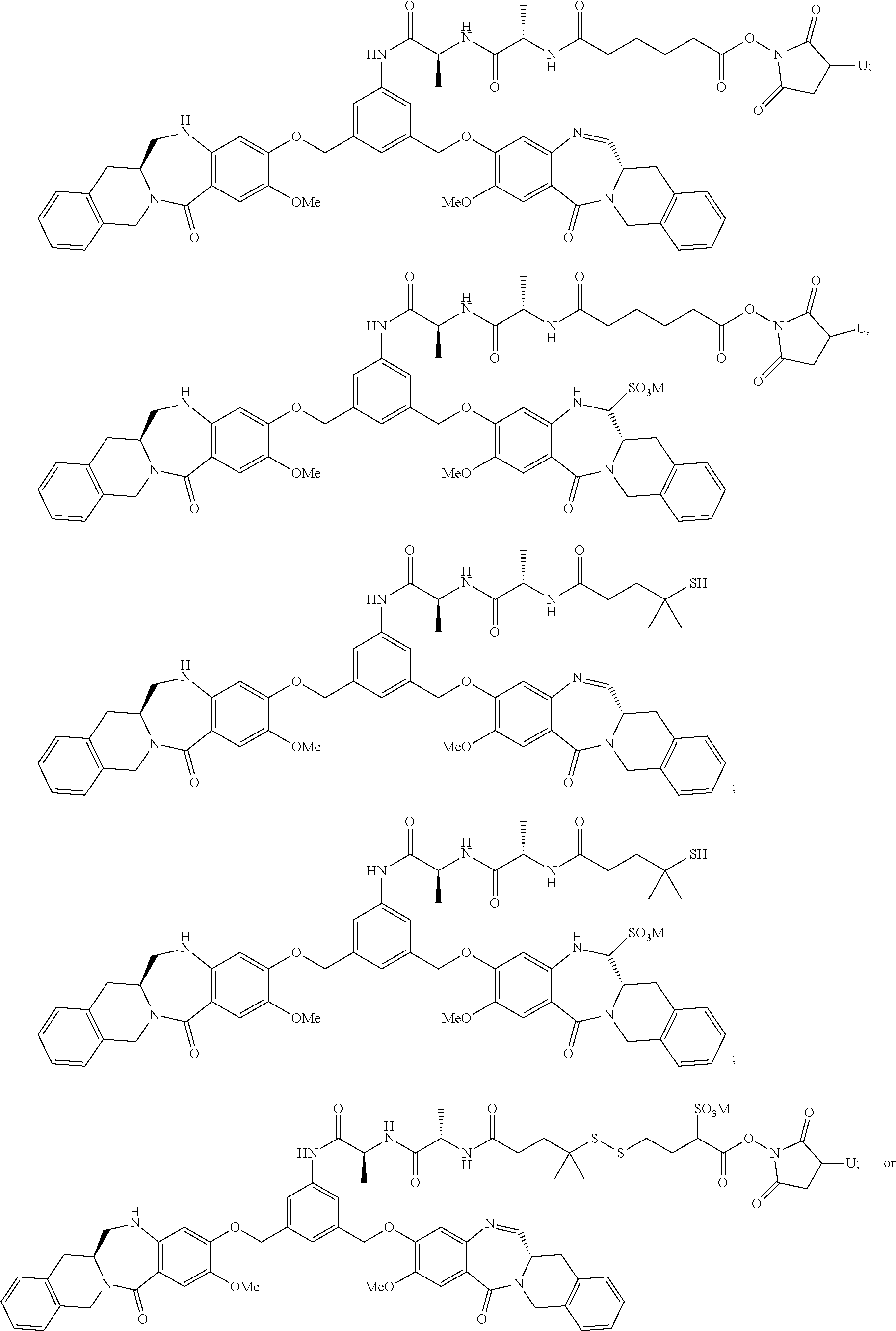 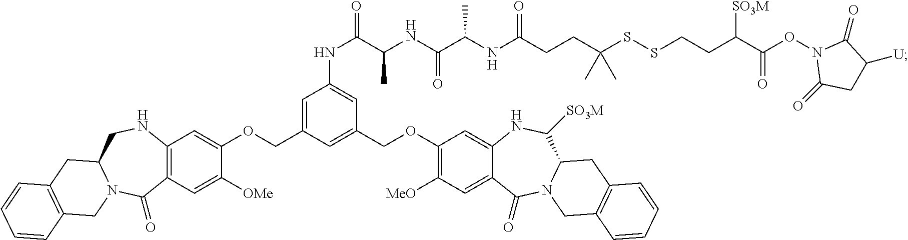
or a pharmaceutically acceptable salt thereof, wherein U is H or SO₃M; and M is H, Na⁺ or K⁺.

23. A cell-binding agent-cytotoxic agent conjugate comprising a cell-binding agent (CBA), covalently linked to a cytotoxic agent, wherein the conjugate is represented by the following formula:
CBACy)_{w} (III),
or a pharmaceutically acceptable salt thereof, wherein:
CBA is a cell-binding agent;
Cy is a cytotoxic agent represented by the following formula: 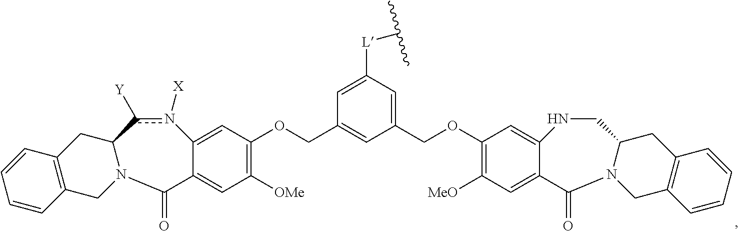
or a pharmaceutically acceptable salt thereof, wherein:
the double line [symbol] between N and C represents a single bond or a double bond, provided that when it is a double bond, X is absent and Y is —H or a (C₁-C₄)alkyl; and when it is a single bond, X is —H or an amine protecting moiety, and Y is —OH or —SO₃M;
L′ is represented by the following formula:
—NR₅—P—C(═O)—W-J′ (L1′);
—NR⁵—P—C(═O)—W—S—Z^{s1} (L2′);
—N(R^{e′})—W—S—Z^{s1} (L3′);
—N(R^{e})—C(═O)—W—S—Z^{s1} (L4′); or
—N(R^{e})—W-J′ (L5′);
R₅, for each occurrence, is independently H or a (C₁-C₃)alkyl;
W is a spacer unit;
J′ is a linking moiety;
R^{e} is H or a (C₁-C₃)alkyl;
R^{e′} is —(CH₂—CH₂—O)ₙ—R^{k};
n is an integer from 2 to 6;
R^{k} is H or Me;
Z^{s1} is a bifunctional linker covalently linked to the cytotoxic agent and the CBA;
w is an integer from 1 to 20.

24-50. (canceled)

51. A pharmaceutical composition comprising the conjugate of claim 23 and a pharmaceutically acceptable carrier.

52. A method of inhibiting abnormal cell growth or treating a proliferative disorder, an autoimmune disorder, destructive bone disorder, infectious disease, viral disease, fibrotic disease, neurodegenerative disorder, pancreatitis or kidney disease in a mammal, comprising administering to said mammal a therapeutically effective amount of a compound of claim 23, and optionally, a chemotherapeutic agent.

53-55. (canceled)


SEQUENCE LISTING (33 sequences)

SEQ ID NO: 1 - protein, 4 aa, Artificial Sequence
  Description of Artificial Sequence Synthetic peptide
ALAL

SEQ ID NO: 2 - protein, 4 aa, Artificial Sequence
  Description of Artificial Sequence Synthetic peptide
  MOD_RES (1)..(1): Beta-Ala
ALAL

SEQ ID NO: 3 - protein, 4 aa, Artificial Sequence
  Description of Artificial Sequence Synthetic peptide
GFLG

SEQ ID NO: 4 - protein, 5 aa, Artificial Sequence
  Description of Artificial Sequence Synthetic peptide
GYFMN

SEQ ID NO: 5 - protein, 17 aa, Artificial Sequence
  Description of Artificial Sequence Synthetic peptide
  MOD_RES (14)..(14): Lys, Gln, His, or Arg
  MOD_RES (16)..(16): Gln, His, Asn, or Arg
  MOD_RES (17)..(17): Gly, Glu, Thr, Ser, Ala, or Val
RIHPYDGDTFYNQXFXX

SEQ ID NO: 6 - protein, 9 aa, Artificial Sequence
  Description of Artificial Sequence Synthetic peptide
YDGSRAMDY

SEQ ID NO: 7 - protein, 15 aa, Artificial Sequence
  Description of Artificial Sequence Synthetic peptide
KASQSVSFAGTSLMH

SEQ ID NO: 8 - protein, 7 aa, Artificial Sequence
  Description of Artificial Sequence Synthetic peptide
RASNLEA

SEQ ID NO: 9 - protein, 9 aa, Artificial Sequence
  Description of Artificial Sequence Synthetic peptide
QQSREYPYT

SEQ ID NO: 10 - protein, 17 aa, Artificial Sequence
  Description of Artificial Sequence Synthetic peptide
RIHPYDGDTFYNQKFQG

SEQ ID NO: 11 - protein, 448 aa, Artificial Sequence
  Description of Artificial Sequence Synthetic polypeptide
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 218 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 118 aa, Artificial Sequence
  Description of Artificial Sequence Synthetic polypeptide
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 15 - protein, 112 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 16 - protein, 112 aa, Artificial Sequence
  Description of Artificial Sequence Synthetic polypeptide
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 17 - protein, 445 aa, Artificial Sequence
  Description of Artificial Sequence Synthetic polypeptide
QVQLQESGPGLVKPSETLSLTCTVSGLSLASNSVSWIRQPPGKGLEWMGVIWNHGGTDYNPSIKSRLSISRDTSKSQVFLKMNSLTAADTAMYFCVRKGGIYFDYWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 213 aa, Artificial Sequence
  Description of Artificial Sequence Synthetic polypeptide
DTVLTQSPSLAVSPGERATISCRASESVSTLMHWYQQKPGQQPKLLIYLASHRESGVPARFSGSGSGTDFTLTIDPMEAEDTATYYCQQSRNDPWTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 448 aa, Artificial Sequence
  Description of Artificial Sequence Synthetic polypeptide
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 214 aa, Artificial Sequence
  Description of Artificial Sequence Synthetic polypeptide
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 214 aa, Artificial Sequence
  Description of Artificial Sequence Synthetic polypeptide
DIQMTQSPSSLSASVGDRVTITCKASQDINNYLAWYQHKPGKGPKLLIHYTSTLHPGIPSRFSGSGSGRDYSFSISSLEPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 450 aa, Artificial Sequence
  Description of Artificial Sequence Synthetic polypeptide
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 211 aa, Artificial Sequence
  Description of Artificial Sequence Synthetic polypeptide
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 447 aa, Artificial Sequence
  Description of Artificial Sequence Synthetic polypeptide
QAQLVQSGAEVVKPGASVKMSCKASGYTFTSYNMHWVKQTPGQGLEWIGYIYPGNGATNYNQKFQGKATLTADTSSSTAYMQISSLTSEDSAVYFCARGDSVPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 213 aa, Artificial Sequence
  Description of Artificial Sequence Synthetic polypeptide
EIVLTQSPATMSASPGERVTITCSAHSSVSFMHWFQQKPGTSPKLWIYSTSSLASGVPARFGGSGSGTSYSLTISSMEAEDAATYYCQQRSSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 447 aa, Artificial Sequence
  Description of Artificial Sequence Synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 219 aa, Artificial Sequence
  Description of Artificial Sequence Synthetic polypeptide
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 214 aa, Artificial Sequence
  Description of Artificial Sequence Synthetic polypeptide
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 444 aa, Artificial Sequence
  Description of Artificial Sequence Synthetic polypeptide
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 444 aa, Artificial Sequence
  Description of Artificial Sequence Synthetic polypeptide
QVQVQESGPGLVAPSQTLSITCTVSGFSLTTSGVSWVRQPPGKGLEWLGVIWGDGSTNYHSSLKSRLSIKKDHSKSQVFLKLNSLTAADTATYYCAKGGYSLAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 213 aa, Artificial Sequence
  Description of Artificial Sequence Synthetic polypeptide
EIVLTQSPATMSASPGERVTMTCSATSSVTYMHWYQQKPGQSPKRWIYDTSNLPYGVPARFSGSGSGTSYSLTISSMEAEDAATYYCQQWSDNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 449 aa, Artificial Sequence
  Description of Artificial Sequence Synthetic polypeptide
QVQLQESGPGLLKPSQSLSLTCTVSGYSITSGFAWHWIRQHPGNKLEWMGYILYSGSTVYSPSLKSRISITRDTSKNHFFLQLNSVTAADTATYYCARGYYGYGAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 4 aa, Unknown
  Description of Unknown KDEL sequence
KDEL